(19) United States
(12) Patent Application Publication
(10) Pub. No.: US 2025/0049480 A1
(43) Pub. Date: Feb. 13, 2025

(54) OSTEOTOMY SYSTEMS AND TECHNIQUES FOR METATARSAL CORRECTION WITH INTRAMEDULLARY INSERTION BODY

(71) Applicant: Treace Medical Concepts, Inc., Ponte Vedra, FL (US)

(72) Inventors: Jonathan M. Dewey, Ponte Vedra, FL (US); Caitlin DiGeorgio, Ponte Vedra, FL (US); Adriaan Kuyler, Ponte Vedra, FL (US); Jason May, St. John’s, FL (US); Mitch Read, Berlin, MA (US); Sean F. Scanlan, Jacksonville, FL (US)

(21) Appl. No.: 18/773,567

(22) Filed: Jul. 15, 2024

Related U.S. Application Data
(60) Provisional application No. 63/513,728, filed on Jul. 14, 2023.
(60) Provisional application No. 63/625,739, filed on Jan. 26, 2024.

(51) Int. Cl. A61B 17/72; A61B 17/80

(52) U.S. Cl. CPC A61B 17/7291; A61B 17/8061

(57) ABSTRACT

Systems, devices, and techniques can be used to perform an osteotomy on a bone of a foot, to realign a cut bone portion relative to an adjacent bone portion, and/or to fixate a moved position of the cut bone portion relative to the adjacent bone portion for fusion. In some examples, the disclosed devices and techniques can be used as part of a metatarsal correction procedure in which a metatarsal is treated to correct a bunion deformity.

### CROSS-REFERENCE

This application claims the benefit of U.S. Provisional Patent Application No. 63/513,728, filed Jul. 14, 2023, and of U.S. Provisional Patent Application No. 63/625,739, filed Jan. 26, 2024. The entire contents of either of these applications is incorporated herein by reference.

### TECHNICAL FIELD

This disclosure relates generally to osteotomy devices, systems, and techniques, and more particularly, to devices, systems, and techniques for performing an osteotomy on a bone of a foot and repositioning a cut bone portion.

### BACKGROUND

Bones within the human body, such as bones in the foot, may be anatomically misaligned. For example, one common type of bone deformity is hallux valgus, which is a progressive foot deformity in which the first metatarsophalangeal joint is affected and is often accompanied by significant functional disability and foot pain. The metatarsophalangeal joint is laterally deviated, resulting in an abduction of the first metatarsal while the phalanges adduct. This often leads to development of soft tissue and a bony prominence on the medial side of the foot, which is called a bunion.

Surgical intervention may be used to correct a bunion deformity. A variety of different surgical procedures exist to correct bunion deformities and may involve removing the abnormal bony enlargement on the first metatarsal and/or attempting to realign the first metatarsal relative to the adjacent metatarsal. In some applications, an osteotomy is performed that involves cutting the metatarsal into two portions and shifting the cut distal portion laterally to reduce the prominence of the bunion. Surgical instruments that can facilitate efficient, accurate, and reproducible clinical results are useful for practitioners performing osteotomy and other bone realignment techniques.

### SUMMARY

In general, this disclosure is directed to devices, systems, and techniques for performing an osteotomy on one or more bones in the foot of the patient. The described devices, systems, and techniques can be utilized to partially or fully correct an anatomical misalignment of the one or more bones. Example instruments and techniques described in the present disclosure can be used to surgically access and cut a bone into two different portions, controllably realign one bone portion relative to the other bone portion, and/or fixate a moved position of the one bone portion relative to the other bone portion.

In some implementations, a clinician surgically accesses a bone of a foot, such as metatarsal of the foot. The metatarsal may be a first metatarsal or a lesser metatarsal, such as a fifth metatarsal. The clinician may utilize an incision guide placed over the skin of the patient to identify a correct location for cutting through the skin to surgically access a location along the foot where the bone is to be cut. The incision guide may be used to mark the location for the incision and/or to guide a cutting instrument to make the incision through the skin of the patient.

In some applications, the clinician inserts a wire percutaneously (through the skin of the patient) into the bone to be cut and then aligns an incision guide with a portion of the wire projecting out of the bone. Use of the incision guide can help ensure that the incision is small and precisely positioned relative to the foot, helping to achieve a minimally invasive procedure.

Independent of whether the clinician utilizes an incision guide, the clinician can surgically access the bone and cut the bone into at least two portions: a distal portion which may be referred to as a capital fragment and a proximal portion. The clinician may attach a cut guide to the bone at a location proximal of a cut location and also distal to the cut location prior to using the cut guide to cut the bone into two portions. For example, the clinician may pin the cut guide using one or more pin receiving holes associated with the cut guide at a location distal of where the bone is to be cut and also pin the cut guide using one or more pin receiving holes associated with the cut guide at a location proximal of where the bone is to be cut. The clinician can then use a guide surface of the cut guide to guide a cutting instrument to cut the bone into the two portions.

With the bone cut into two portions, the clinician can move the distal portion relative to the proximal portion, e.g., to help correct an anatomical deformity. For example, the clinician can shift the distal portion in the transverse plane (e.g., move the distal portion laterally), rotate the distal portion in the frontal plane, and/or shift the distal portion in the sagittal plane. In some implementations, the clinician engages the distal portion with a bone positioning device operable to controllably move the distal portion relative to the proximal portion.

Before, after, and/or while moving the distal portion of the cut bone relative to the proximal portion in one or more planes, the clinician may engage one or more fixation devices with the proximal and/or distal portions. The one or more fixation devices may take the form of plates, screws, staples, and/or intramedullary implants. In some examples, a fixation device is used that is an intramedullary implant having a shaft insertable into the medullary canal of one or both of the proximal and distal bone portions. For example, the intramedullary implant may have a shaft insertable into the medullary canal of one of the proximal or distal bone portions and one or more apertures positionable over an external surface (cortical bone) of the other of the proximal and distal bone portions. The one or more apertures can receive fixation elements (e.g., one or more screws) insertable therethrough to fix the implant to the underlying bone.

For example, the intramedullary implant may have a shaft configured to be inserted into the medullary canal of the proximal bone portion and a plate connected to the shaft positionable over the external surface of the distal bone portion. The plate may define one or more screw receiving apertures through which one or more corresponding screws can be inserted to affix the plate to the underlying distal bone portion. The shaft of the intramedullary implant may or may not also include one or more screw receiving apertures through which one or more corresponding screws can be inserted. A fixation device, such as an intramedullary implant, usable in an osteotomy procedure according to the disclosure can have a variety of different configurations as described herein.

In some examples, the osteotomy procedure utilizes an insertion instrument to guide positioning of the intramedullary implant relative to the proximal bone portion and/or the distal bone portion. The insertion instrument can releasably engage the implant and be used by the clinician to guide the implant into the target bone portion receiving the intramedullary shaft of the implant. In some examples, the insertion instrument defines a handle through which a clinician can provide an axially-directed force in a direction generally parallel to the length of the intramedullary shaft to help insert the intramedullary shaft into the bone portion. Depending on the configuration of the insertion instrument, the clinician may detach the insertion instrument from the intramedullary implant before or after inserting one or more fixation devices (e.g., screws) through corresponding fixation apertures of the implant.

In some implementations, an insertion guide connectable to an intramedullary implant defines one or more guide apertures defining openings axially aligned with one or more fixation apertures of the intramedullary implant. A guide aperture of the instrument can be used to guide insertion of a screw into a corresponding fixation aperture of the implant, e.g., when the implant is positioned at a desired location relative to the proximal and/or distal bone portion. The clinician can guide the screw(s) directly through the guide aperture defined by the instrument, pre-drill a hole using the guide aperture, and/or or may insert a wire through the guide aperture and then guide the screw via the wire (e.g., a cannulated screw) after detaching the instrument from the implant. Configuring the insertion instrument with one or more guide apertures corresponding to the location of one or more fixation apertures of the implant can be useful to help the clinician efficiently and accurately engage the implant with one or more corresponding fixation devices.

In some examples, an insertion guide connectable to an intramedullary implant may additionally or alternatively include a positioning device that is operable to control the positioning of one bone portion relative to another bone portion and/or to control the positioning of the implant relative to a bone portion. In some examples, the positioning device is operable to apply a force causing the distal bone portion to move laterally, e.g., helping to control positioning of the distal bone portion relative to the proximal bone portion. For example, when implemented to insert the shaft of the intramedullary implant into the proximal bone portion, the insertion instrument may include one or more bone positioning devices that apply a force biasing the distal bone portion in the transverse plane (e.g., laterally) and/or in the frontal plane. In some examples, the bone positioning device applies a force to a medial side of the proximal bone portion, causing a distal portion of the insertion instrument to rotate laterally and push the distal bone portion laterally. Applying a force with the bone positioning device can also help secure/stabilize the entire instrument on the foot, +e.g., allowing the clinician to visualize the foot and instrument under fluoroscopy to determine whether the distal bone portion has been adequately realigned or whether further correction is desired. The same or a different bone positioning device may apply a force to the distal bone portion (e.g., via a pin inserted into the bone portion), causing a distal bone portion to rotate in the frontal plane.

In one example, an intramedullary implant insertion and positioning instrument is described that includes a body, a bone positioning device operatively connected to the body and an intramedullary insertion. The bone positioning device is configured to engage a bone portion underlying the body and to apply a force between the body and bone portion. The intramedullary insertion body is configured to be inserted into a medullary cannel of the bone portion, the intramedullary insertion body being operatively connected to the body and movable relative to the body to apply a force pulling the bone portion toward the body. In some implementations of this example, the intramedullary insertion body is positionable adjacent an intramedullary implant carried by the body.

In some implementations of this example, the body includes an intramedullary implant receiving surface against which an intramedullary implant is configured to positioned. For example, the body may include an insertion aperture configured to receive an attachment rod for releasably coupling the intramedullary implant to the body. The intramedullary insertion body can have an opening through which the attachment rod is configured to extend to couple the intramedullary implant to the body through the intramedullary insertion body. In some implementations of the example, the body further includes a hook configured to wrap at least partially about the intramedullary implant, when the intramedullary implant is attached to the body. In some implementations of the example, the instrument further includes a detachable guide body configured to be detachably connected to the body. For example, the detachable guide body may include one or more screw insertion apertures configured to align with a trajectory of one or more fixation apertures of an intramedullary implant, when the intramedullary implant is attached to the body.

In another example, intramedullary implant insertion and positioning instrument is described that includes a body, a first bone positioning device operatively connected to the body, and a second bone positioning device operatively connected to the body. The first bone positioning device being configured to engage a bone portion underlying the body and to apply a force between the body and bone portion to move the bone portion in a transverse plane. The second bone positioning device is operatively connected to the body. The second bone positioning device is configured to engage the bone portion and to apply a force between the body and the bone portion to move the bone portion in a frontal plane. In some implementations of the example, the instrument further includes an intramedullary insertion body configured to be inserted into a medullary cannel of the bone portion, the intramedullary insertion body being operatively connected to the body and movable relative to the body to apply a force pulling the bone portion toward the body. In some implementations of the example, the second bone positioning device includes a body defining at least one wire receiving opening configured to engage a wire inserted into the bone portion. The second bone positioning device may include an arm extending laterally and distally from the body, such as an arcuate arm defining a channel.

In one example, a bunion treatment method is described that involves cutting a metatarsal bone of a foot into a first metatarsal portion and a second metatarsal portion and inserting a stem portion of an intramedullary implant into the first metatarsal portion and positioning at least one fixation aperture extending through a plate portion of the intramedullary implant overlying the second metatarsal portion. The example specifies that the stem portion has a length extending from a first end to a second end with the plate portion being positioned at the second end of the stem portion. The example also specifies that the stem portion extends at an angle relative to the plate portion, and inserting the stem portion of the intramedullary implant into the first metatarsal portion involves positioning the first end of the stem portion in contact with a lateral cortical wall of the first metatarsal portion. The example also involves inserting a fixation member through the at least one fixation aperture extending through the plate portion of the intramedullary implant and into the second metatarsal portion to fixate a moved position of the second metatarsal portion relative to the first metatarsal portion.

In another example, an intramedullary implant is described that includes a stem portion configured to be inserted into a medullary canal of a first metatarsal portion, where the stem portion has a length extending from a first end to a second end. The intramedullary implant also includes a plate portion at the second end of the stem portion and configured to be positioned against a medial side of a second metatarsal portion. The plate portion has at least one fixation aperture. The example species that the stem portion extends at an angle relative to the plate portion and is configured to be inserted into the medullary canal of the first metatarsal bone with the first end of the stem portion in contact with a lateral cortical wall of the first metatarsal portion.

In another example, an intramedullary implant insertion and positioning instrument is described that includes a body and an intramedullary insertion body. The example specifies that the body is configured to releasably connect to an intramedullary implant having a stem portion insertable into a first metatarsal portion and a plate portion positionable against a second metatarsal portion. The example also specifies that the intramedullary insertion body is configured to be inserted into a medullary canal of the first metatarsal portion, the intramedullary insertion body is operatively connected to the body, and the intramedullary insertion body and the intramedullary implant are movable relative to each other, when the intramedullary implant is releasably connected to the body.

In another example, a method is described that involves cutting a metatarsal bone of a foot into a first metatarsal portion and a second metatarsal portion and inserting a stem portion of an intramedullary implant releasably connected to a body of an instrument and an intramedullary insertion body connected to the body of the instrument into a medullary canal of the first metatarsal portion. The method includes positioning a plate portion of the intramedullary implant overlying the second metatarsal portion and moving the intramedullary insertion body relative to the intramedullary implant to apply a force to the first metatarsal portion. The method also includes inserting a fixation member through a fixation aperture extending through the plate portion of the intramedullary implant and into the second metatarsal portion to fixate a moved position of the second metatarsal portion relative to the first metatarsal portion.

In another example, an intramedullary implant insertion and positioning instrument is described that includes a body configured to releasably connect to an intramedullary implant having a stem portion insertable into a first metatarsal portion and a plate portion positionable against a second metatarsal portion. The example instrument also includes a bone positioning device operatively connected to the body, the bone positioning device being configured to engage a pin inserted into the second metatarsal portion and apply a force to the pin to move the second metatarsal portion in at least a frontal plane.

In another example, a method is described that involves cutting a metatarsal bone of a foot into a first metatarsal portion and a second metatarsal portion. The method also includes inserting a stem portion of an intramedullary implant releasably connected to a body of an instrument into a medullary canal of the first metatarsal portion and positioning a plate portion of the intramedullary implant overlying the second metatarsal portion. The method further includes inserting a pin into the second metatarsal portion and using a bone positioning device operatively connected to the body of the instrument to engage the pin inserted into the second metatarsal portion and apply a force to the pin to move the second metatarsal portion in at least a frontal plane. The method also includes inserting a fixation member through a fixation aperture extending through the plate portion of the intramedullary implant and into the second metatarsal portion to fixate a moved position of the second metatarsal portion relative to the first metatarsal portion.

In another example, an intramedullary implant insertion and positioning instrument is described that includes a body configured to releasably connect to an intramedullary implant having a stem portion insertable into a first metatarsal portion and a plate portion positionable against a second metatarsal portion. The method also includes a bone positioning device operatively connected to the body, the bone positioning device being configured to apply a force between the body and a bone portion underlying the body.

In another example, a method is described that includes cutting a metatarsal bone of a foot into a first metatarsal portion and a second metatarsal portion and inserting a stem portion of an intramedullary implant into the first metatarsal portion and positioning a fixation aperture extending through a plate portion of the intramedullary implant overlying the second metatarsal portion. The method also includes using a bone positioning device releasably connected to the intramedullary implant to move the second metatarsal portion relative to the first metatarsal portion in at least one plane. The method further includes inserting a fixation member through the fixation aperture extending through the plate portion of the intramedullary implant and into the second metatarsal portion to fixate a moved position of the second metatarsal portion relative to the first metatarsal portion.

The details of one or more examples are set forth in the accompanying drawings and the description below. Other features, objects, and advantages will be apparent from the description and drawings, and from the claims.

### BRIEF DESCRIPTION OF DRAWINGS

FIGS. 1A and 1B are front views of a foot showing a normal first metatarsal position and an example frontal plane rotational misalignment position, respectively.

FIGS. 2A and 2B are top views of a foot showing a normal first metatarsal position and an example transverse plane misalignment position, respectively.

FIGS. 3A and 3B are side views of a foot showing a normal first metatarsal position and an example sagittal plane misalignment position, respectively.

FIG. 4 is a flow diagram illustrating an example technique for performing an osteotomy and bone realignment procedure.

FIG. 5A is a medial-to-lateral view fluoroscopic image of an example portion of a foot showing a radiopaque K-wire positioned along the midline of the foot to identify the midline location.

FIG. 5B is a dorsal-to-planar view fluoroscopic image of an example portion of the foot from FIG. 5A showing the positioning of a radiopaque wire inserted into the first MTP joint.

FIG. 6 shows a medial side of a foot with the midline of the first metatarsal marked along the skin by a midline marking line and the MTP joint marked along the skin by a MTP marking line.

FIG. 7A illustrates one example bone preparation guide that may be used as part of an osteotomy procedure.

FIG. 7B is a perspective illustration of another example configuration of a bone preparation guide that may be used as part of an osteotomy procedure.

FIG. 7C illustrates the bone preparation guide of FIG. 7B positioned adjacent a first metatarsal.

FIGS. 7D and 7E illustrate the bone preparation guide of FIG. 7B being advanced over wire.

FIG. 7F illustrates the bone preparation guide of FIG. 7B being secured to a first metatarsal.

FIG. 7G illustrates the bone preparation guide of FIG. 7B with orienting wire removed from the guide.

FIG. 7H is an illustration of an example configuration of a bone preparation guide that includes a sidewall cutout.

FIG. 7I is a perspective illustration of an example configuration of a bone preparation guide configured for use with a burr.

FIG. 7J is a perspective illustration of an example configuration of a bone preparation guide having a block sized relative to a plate portion of an intramedullary implant.

FIG. 8 is a perspective view of another example configuration of a bone preparation guide.

FIG. 9 is a side view of the example bone positioning guide from FIG. 7A illustrating an example angular orientation of pin receiving holes.

FIG. 10 illustrates an example bone preparation guide installed over a K-wire on an example foot of a patient.

FIGS. 11A and 11B are side and top views, respectively, of an example broach instrument.

FIG. 12 is a fluoroscopic image showing an example broach instrument inserted into a proximal metatarsal bone portion.

FIGS. 13A-13C are different views of an example intramedullary implant.

FIGS. 13D and 13E are a perspective view and a back view, respectively, of another example configuration of an intramedullary implant.

FIG. 13F is a perspective of an example configuration of the intramedullary implant from FIGS. 13D and 13E where the intramedullary implant includes a fixation aperture in the tail portion of the intramedullary stem.

FIG. 13G is a perspective view of the intramedullary implant from FIG. 13F illustrating an example tack that can be used to form an opening for inserting a tail screw.

FIGS. 14 and 15 are top and side views, respectively, of another example configuration of an intramedullary implant.

FIGS. 16A-16F are views of different example inserter configurations that can be used with an intramedullary implant.

FIG. 17 is an example dorsal view fluoroscopic image showing an example configuration of an implant inserted into the metatarsal bone portion.

FIG. 18 is an example dorsal view fluoroscopic image showing an example drill inserted into fixation aperture of an intramedullary implant.

FIG. 19 is an example dorsal view fluoroscopic image showing a K-wire inserted through a fixation aperture 88 of an intramedullary implant.

FIG. 20 is a perspective view of an example bone positioning device that can be used to control the positioning of a distal metatarsal bone portion relative to a proximal metatarsal bone portion.

FIG. 21 is an example dorsal view fluoroscopic image showing screws inserted through fixation apertures of an example intramedullary implant.

FIGS. 22A and 22B are a perspective view and a sectional view of an example instrument that can be used to guide installation of an intramedullary implant and/or positioning of a distal metatarsal bone portion relative to a proximal metatarsal bone portion.

FIG. 23A is a perspective view of an example configuration of implant attachment member that can be used on the instrument of FIGS. 22A and 22B.

FIG. 23B is a perspective view of an example configuration of an intramedullary implant.

FIG. 23C is a side sectional view of an example implant engagement member grasping opposed sidewalls of an intramedullary implant.

FIGS. 24A and 24B illustrate example procedural steps in which a bone positioning device facilitate installation of an intramedullary implant and/or positioning of a distal metatarsal bone portion relative to a proximal metatarsal bone portion.

FIG. 24C is an example image of a foot showing an intramedullary implant fixated to proximal and distal metatarsal bone portions.

FIGS. 25A-25C illustrate example screw trajectory configurations that can be used for an intramedullary implant.

FIG. 26A illustrates a configuration of the example instrument of FIGS. 22A and 22B defining screw insertion apertures aligned with the fixation aperture configuration of the intramedullary implant of FIG. 25A.

FIG. 26B illustrates a configuration of the example instrument of FIGS. 22A and 22B defining screw insertion apertures aligned with the fixation aperture configuration of the intramedullary implant of FIG. 25B.

FIG. 27 is an illustration of another example configuration of an intramedullary implant showing example fixation aperture and screw trajectory orientations.

FIG. 28 illustrates example positioning of fixation apertures on the distal end of an intramedullary implant stem to allow all the fixation apertures of intramedullary implant to be accessed through a single incision location.

FIG. 29 is a side sectional illustration of an example cap-less locking screw configuration with a shallower terminal thread.

FIG. 30 is top view of an alternative configuration of an intramedullary implant where the implant includes a stem that is configured to be deformed and enlarged in response to engaging a fixation device with the stem.

FIG. 31 is top view of the intramedullary implant from FIG. 30 illustrating an example deformation profile.

FIGS. 32 and 33 illustrate an example insertion trajectory for a fixation member through the longitudinal gap in the intramedullary implant resulting in deformation of the implant stem.

FIGS. 34A-34C are different views of another example configuration of an instrument that can be used to guide installation of an intramedullary implant and/or positioning of a distal metatarsal bone portion relative to a proximal metatarsal bone portion.

FIG. 34D is a perspective view of an example configuration of the instrument from FIGS. 34A-34C showing the instrument configured with an adjustable body defining the contact surface of a bone positioning device.

FIG. 34E is a close-up view an example configuration of the body of FIG. 34D.

FIGS. 34F and 34G are top views illustrating example procedural steps in which the instrument with angularly adjustable body of FIG. 34D is engaged with a proximal metatarsal bone portion and a distal metatarsal bone portion.

FIGS. 34H and 34I illustrate example configurations of a contact surface defining body for a bone positioning device defining an asymmetrical configuration.

FIG. 34J illustrates an example configuration of a contact surface defining body for a bone positioning device having an example side edge cutout and example slot.

FIG. 34K illustrates another example configuration of a contact surface defining body for a bone positioning device defining one or more pointed contact surfaces.

FIG. 35A is a side view of the instrument of FIGS. 34A-34C illustrating an example implant attachment arrangement that can be used to attach an intramedullary implant to the instrument.

FIG. 35B is a sectional side view of a portion of the instrument of FIGS. 34A-34C further illustrating example attachment features that can be used to attach an intramedullary implant to the instrument.

FIG. 36 is a perspective view of the instrument of FIGS. 34A-34C illustrating an example detachable guide body that can be detachably connected to a body of the instrument.

FIGS. 37A and 37B are different perspective views of an example configuration of the detachable guide body of FIG. 36.

FIG. 38A illustrates the guide body of FIGS. 37A and 37B being inserted onto a main body of the instrument of FIGS. 34A-34C.

FIG. 38B illustrates the guide body of FIGS. 37A and 37B seated on and operatively engaged with the body of the instrument of FIGS. 34A-34C.

FIGS. 39A-39C are views of an example bone positioning device that can be used with the instrument of FIGS. 34A-34C to control repositioning of a bone portion in the frontal plane.

FIGS. 40A-40C are views of another example bone positioning device that can be used with the instrument of FIGS. 34A-34C to control repositioning of a bone portion in the frontal plane.

FIG. 40D is a perspective view of another example configuration of a bone positioning device operable to apply a force to distal metatarsal bone portion to controllably reposition the bone portion in the frontal plane.

FIGS. 40E and 40F are perspective views of the example components of the bone positioning device of FIG. 40D shown disassembled from each other.

FIG. 40G illustrates an example configuration of the bone positioning device of FIG. 40D attached to and extending from the instrument of FIGS. 34A-34C with the instrument engaged with both a proximal metatarsal bone portion and a distal metatarsal bone portion.

FIGS. 40H and 40I illustrate the example bone positioning device from FIG. 40G showing a wire positioned in different example grooves of a wire receiving body to adjust the sagittal plane angle of a distal metatarsal bone portion.

FIGS. 40J-40Q illustrate example surgical technique steps that can implemented to treat a bunion deformity using example instruments according to the disclosure.

FIGS. 41A-41F are different images of an example loading block that can be used to load an intramedullary implant onto the instrument of FIGS. 34A-34C.

FIGS. 42A and 42B are images of an example configuration of a trial implant and broach can be used to form a pocket in the end of a metatarsal bone portion to prepare the metatarsal bone portion to receive the stem of the intramedullary implant.

FIGS. 43A-43D illustrate example surgical technique steps that can be used when performing a procedure with the trial implant and broach of FIGS. 42A and 42B.

FIGS. 44A-44M illustrate example implant insertion and bone realignment procedure steps that may be performed using an example intramedullary implant and instrument according to the disclosure.

FIGS. 45A-45D illustrate example procedural steps that can be used to introduce a biologic to a joint space using the instrument of FIGS. 34A-34C.

FIGS. 46A and 46B is an angled top view and a partially sectionalized perspective view, respectively, of an example intramedullary insertion body assembly.

FIG. 46C as a partial sectionalized view of an example configuration of the body of an instrument defining male projection features that can be received in grooves of an example intramedullary insertion body assembly.

FIGS. 46D and 46E are side views of an example intramedullary insertion body assembly showing different positions of the assembly relative to the body of an instrument.

FIGS. 46F-46H our different illustrations showing an example cam device inserted through an opening in an intramedullary insertion body and being turned to spread the legs of the assembly apart.

FIGS. 46I-46R illustrates different example intramedullary insertion body configurations that can be provided as an interchangeable system of components.

FIGS. 47A-47G illustrate example procedural steps that may be performed using an example intramedullary implant and instrument according to the disclosure.

FIG. 48 illustrates an example medial projection of a proximal metatarsal bone portion that may desirably be removed.

FIG. 49 illustrates an example removal profile of the medial projection of the proximal metatarsal bone portion.

FIGS. 50A-50I illustrate example guides and associated procedural steps that can be used to guide removal of the medial projection of the proximal metatarsal bone portion.

FIGS. 51A and 51B are illustrations of an example configuration of an instrument in which an intramedullary implant is operatively connected to a body of the instrument is movable relative to the body.

### DETAILED DESCRIPTION

This disclosure generally relates to devices, systems, and techniques for performing a bone osteotomy and realignment procedure in which a bone is cut into at least two portions and one portion is move relative to another portion. In an exemplary applications, the devices and techniques can be used during a surgical procedure performed on one or more bones, such as bones in the foot or hand, where the bones are relatively small compared to bones in other parts of the human anatomy. In one example, a procedure utilizing embodiments of the disclosure can be performed to correct metatarsal misalignment. An example of such a procedure is a bunion correction procedure where an osteotomy is performed on a first metatarsal of the foot to divide the first metatarsal into a proximal portion and a distal portion. The distal portion of the first metatarsal can be moved (e.g., laterally) relative to the proximal portion to reduce or eliminate the bony prominence of the bunion. Another example is a bunionette correction procedure (also known as a tailor's bunion procedure) performed on a fifth metatarsal of the foot to divide the fifth metatarsal into a proximal portion and a distal portion. The distal portion of the fifth metatarsal can be moved (e.g., medially) relative to the proximal portion to reduce or eliminate the bony prominence on the fifth metatarsal.

In some examples, an osteotomy procedure is performed to treat hallux valgus, which is referred to as a bunion. Hallux valgus, also referred to as hallux abducto valgus, is a complex progressive condition that is characterized by lateral deviation (valgus, abduction) of the hallux and medial deviation of the first metatarsophalangeal joint. Hallux valgus typically results in a progressive increase in the hallux abductus angle, the angle between the long axes of the first metatarsal and proximal phalanx in the transverse plane. An increase in the hallux abductus angle may tend to laterally displace the plantar aponeurosis and tendons of the intrinsic and extrinsic muscles that cross over the first metatarsophalangeal joint from the metatarsal to the hallux. Consequently, the sesamoid bones may also be displaced, e.g., laterally relative to the first metatarsophalangeal joint, resulting in subluxation of the joints between the sesamoid bones and the head of the first metatarsal. This can increase the pressure between the medial sesamoid and the crista of the first metatarsal head.

In some examples, an osteotomy procedure is performed to treat a tailor's bunion, also known as digitus quintus varus or bunionette. A bunionette is a callus and an adventitious bursa that overlies a prominent, laterally deviated fifth metatarsal head and a medially deviated fifth toe.

While devices and techniques are generally described herein in connection with the first metatarsal of the foot as part of a bunion correction procedure, the techniques and devices may be used on other bones and/or to treat other bone conditions. In various examples, the devices, systems, and/or techniques of the disclosure may be utilized on comparatively small bones in the foot such as a metatarsal (e.g., first, second, third, fourth, or fifth metatarsal), a cuneiform (e.g., medial, intermediate, lateral), a cuboid, a phalanx (e.g., proximal, intermediate, distal), and/or combinations thereof.

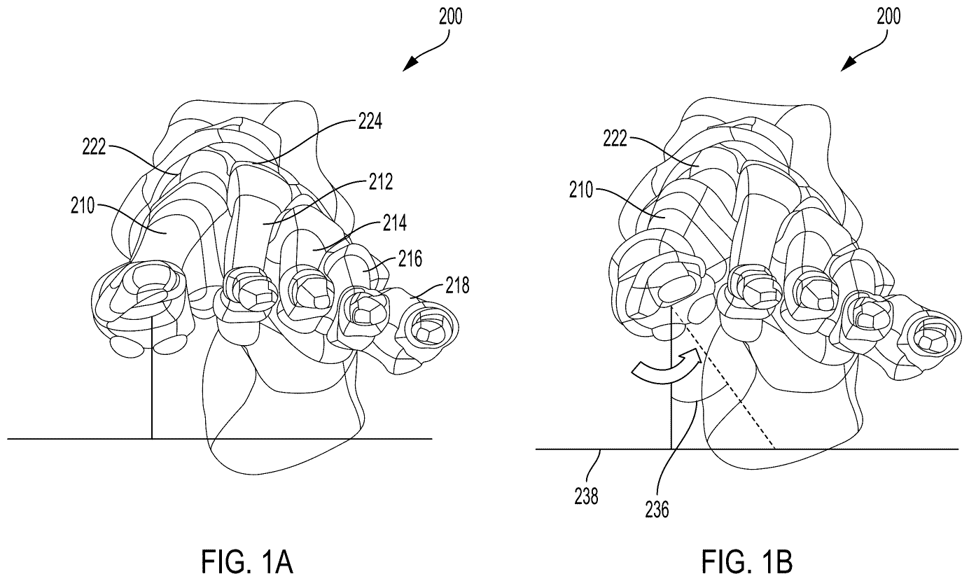

To further understand example techniques of the disclosure, the anatomy of the foot will first be described with respect to FIGS. 1-3 along with example misalignments that may occur and be corrected according to the present disclosure. A bone misalignment may be caused by hallux valgus (bunion), bunionette, a natural growth deformity, and/or other condition.

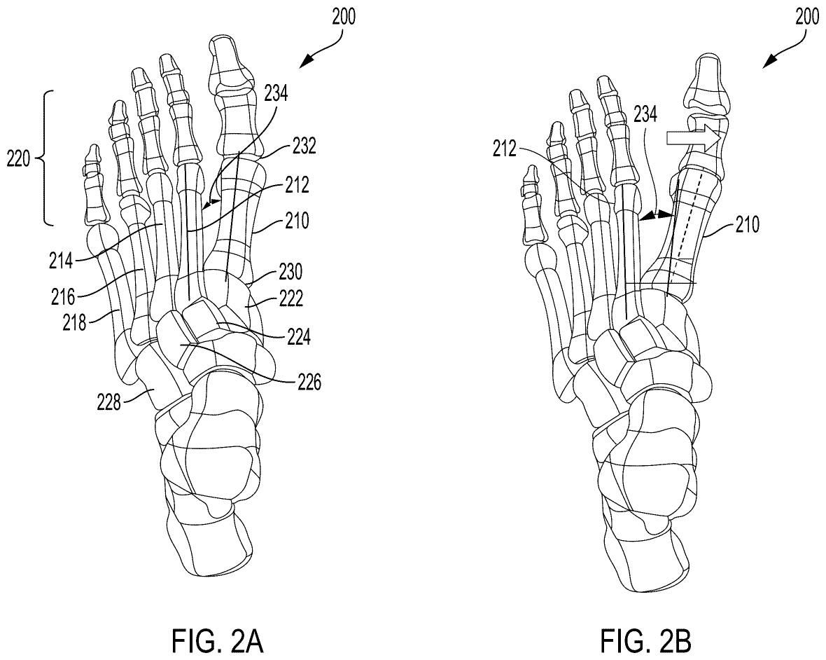

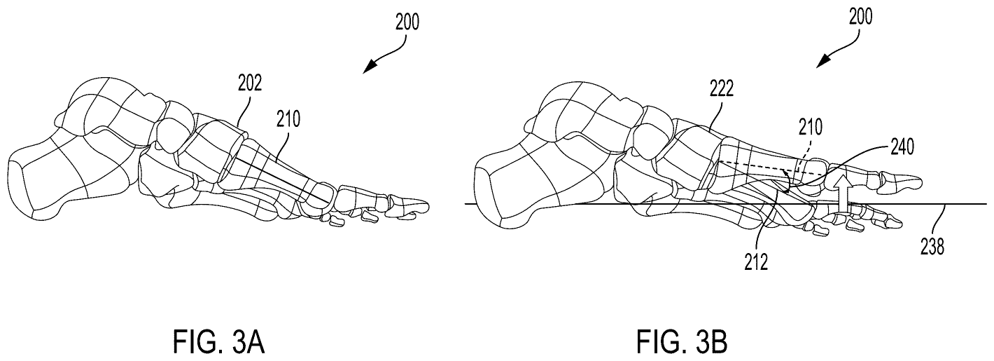

FIGS. 1A and 1B are front views of foot 200 showing a normal first metatarsal position and an example frontal plane rotational misalignment position, respectively. FIGS. 2A and 2B are top views of foot 200 showing a normal first metatarsal position and an example transverse plane misalignment position, respectively. FIGS. 3A and 3B are side views of foot 200 showing a normal first metatarsal position and an example sagittal plane misalignment position, respectively. While FIGS. 1B, 2B, and 3B show each respective planar misalignment in isolation, in practice, a metatarsal may be misaligned in any two of the three planes or even all three planes. Accordingly, it should be appreciated that the depiction of a single plane misalignment in each of FIGS. 1B, 2B, and 3B is for purposes of illustration and a metatarsal may be misaligned in multiple planes that is desirably corrected. Further, a bone condition treated according to the disclosure may not present any of the example misalignments described with respect to FIGS. 1B, 2B, and 3B, and it should be appreciated that the disclosure is not limited in this respect.

With reference to FIGS. 1A and 2A, foot 200 is composed of multiple bones including a first metatarsal 210, a second metatarsal 212, a third metatarsal 214, a fourth metatarsal 216, and a fifth metatarsal 218. The metatarsals are connected distally to phalanges 220 and, more particularly, each to a respective proximal phalanx. The first metatarsal 210 is connected proximally to a medial cuneiform 222, while the second metatarsal 212 is connected proximally to an intermediate cuneiform 224 and the third metatarsal is connected proximally to lateral cuneiform 226. The fourth and fifth metatarsals 216, 218 are connected proximally to the cuboid bone 228. The joint 230 between a metatarsal and respective cuneiform (e.g., first metatarsal 210 and medial cuneiform 222) is referred to as the tarsometatarsal (“TMT”) joint. The joint 232 between a metatarsal and respective proximal phalanx is referred to as a metatarsophalangeal (“MTP”) joint. The angle 234 between adjacent metatarsals (e.g., first metatarsal 210 and second metatarsal 212) is referred to as the intermetatarsal angle (“IMA”).

As noted, FIG. 1A is a frontal plane view of foot 200 showing a typical position for first metatarsal 210. The frontal plane, which is also known as the coronal plane, is generally considered any vertical plane that divides the body into anterior and posterior sections. On foot 200, the frontal plane is a plane that extends vertically and is perpendicular to an axis extending proximally to distally along the length of the foot. FIG. 1A shows first metatarsal 210 in a typical rotational position in the frontal plane. FIG. 1B shows first metatarsal 210 with a frontal plane rotational deformity characterized by a rotational angle 236 relative to ground, as indicated by line 238.

FIG. 2A is a top view of foot 200 showing a typical position of first metatarsal 210 in the transverse plane. The transverse plane, which is also known as the horizontal plane, axial plane, or transaxial plane, is considered any plane that divides the body into superior and inferior parts. On foot 200, the transverse plane is a plane that extends horizontally and is perpendicular to an axis extending dorsally to plantarly (top to bottom) across the foot. FIG. 2A shows first metatarsal 210 with a typical IMA 234 in the transverse plane. FIG. 2B shows first metatarsal 210 with a transverse plane rotational deformity characterized by a greater IMA caused by the distal end of first metatarsal 210 being pivoted medially relative to the second metatarsal 212.

FIG. 3A is a side view of foot 200 showing a typical position of first metatarsal 210 in the sagittal plane. The sagittal plane is a plane parallel to the sagittal suture which divides the body into right and left halves. On foot 200, the sagittal plane is a plane that extends vertically and is perpendicular to an axis extending proximally to distally along the length of the foot. FIG. 3A shows first metatarsal 210 with a typical rotational position in the sagittal plane. FIG. 3B shows first metatarsal 210 with a sagittal plane rotational deformity characterized by a rotational angle 240 relative to ground, as indicated by line 238.

Standard medical planes of reference and descriptive terminology are employed in this disclosure. A sagittal plane divides a body into right and left portions. A coronal or frontal plane divides a body into anterior and posterior portions. A transverse plane divides a body into superior and inferior portions. Anterior means toward the front of a body. Posterior means toward the back of a body. Superior or cephalad means toward the head. Inferior or caudal means toward the feet or tail. Medial means toward the midline of a body (e.g., toward a plane of bilateral symmetry of the body). Lateral means away from the midline of a body or away from a plane of bilateral symmetry of the body. Proximal means toward the trunk of the body. Distal means away from the trunk. Dorsal means toward the top of the foot or other body structure. Plantar means toward the sole of the foot or toward the bottom of the body structure.

Surgical techniques and instruments according to the disclosure can be useful to treat a misalignment of one or more bones of the foot, such as first metatarsal 210. In some applications, the technique involves surgically accessing first metatarsal 210. The clinician may utilize an incision guide to identify the location and size of the incision to be made relative to first metatarsal 210 prior to making the incision through the skin of the patient to surgically access the bone. After making the incision through the skin of the patient, the clinician may attach a cutting guide, also referred to as a bone preparation guide, having one or more guide surfaces configured to guide a cutting instrument. The clinician can use the cutting guide to guide the cutting instrument to cut the first metatarsal into a distal portion (which can be referred to as a capital fragment) and a residual proximal portion.

With the first metatarsal cut into two portions, the distal portion can be realigned in one or more planes relative to the proximal portion to reduce or eliminate an anatomic misalignment. For example, the distal portion can be realigned in two or more planes, or three planes relative to the proximal portion. In some examples, the distal portion is moved laterally in a transverse plane relative to the proximal portion (e.g., to reduce the bony prominence associated with the bunion deformity), the distal portion is rotated in a frontal plane relative to the proximal portion (e.g., to reposition the sesamoid bones plantarly under the distal portion), and/or the distal portion is plantar flexed or dorsiflexed in the sagittal plane. The repositioning of the distal portion can occur via the clinician's hand (e.g., grasping one or more wires inserted into the distal portion) and/or with the aid of instrumentation that applies a force in one or more planes to control repositioning of the distal portion. The clinician can install a fixation device across the osteotomy location between the distal portion and proximal portion to fixate a moved position of the distal portion relative to the proximal portion. The fixation device can hold the moved position of the distal portion to allow bone to form and grow between the proximal portion and moved distal portion, thereby fusing the two portions together. The clinician may utilize one or more instruments, implants, and/or techniques according to disclosure to perform the osteotomy, bone realignment, and/or fixation of the realigned bone portions.

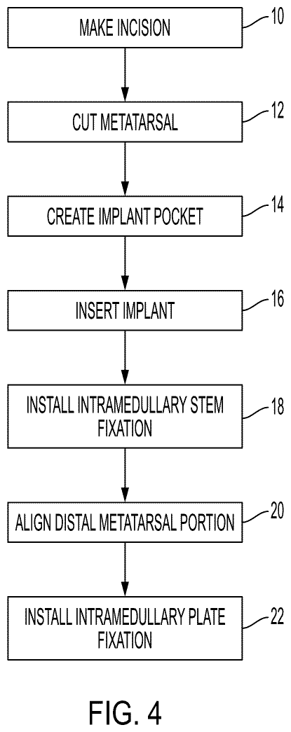

FIG. 4 is a flow diagram illustrating an example technique for performing an osteotomy and bone realignment procedure. The example technique will be described with respect to first metatarsal 210, although can be performed on other bones, as discussed above.

The example technique of FIG. 4 involves surgically accessing first metatarsal 210 (step 10 on FIG. 4). To surgically access the bone, the patient may be placed in a supine position on the operating room table and general anesthesia or monitored anesthesia care administered. Hemostasis can be obtained by applying thigh tourniquet or mid-calf tourniquet.

The clinician may image at least a portion of the foot 200 where first metatarsal 210 is to be cut and, correspondingly, the incision is to be made of. The clinician may take a fluoroscopic images of at least a portion of foot 200 in one or more views encompassing the region where first metatarsal 210 is to be cut. The clinician can identify the midline of first metatarsal 210 (midline between the dorsal-most surface and the planter-most surface) on the medial side of the foot based on the imaging. The clinician may position a K-wire or other radiopaque instrument along the midline while viewing the foot under imaging to identify the midline location. The clinician can also identify the metaphysis region, e.g., the first MTP joint 232 between metatarsal 210 and proximal phalanx 220. The clinician may position a K-wire or other radiopaque instrument at the MTP joint while viewing the foot under imaging to identify the MTP joint. For purposes of this disclosure, the terms wire and pin are used interchangeably and generally refer to an elongated member having a length greater than a width; the cross-sectional shape of the wire or pin may typically be circular (although other shapes can be used) and may or may not be constant across the length of the wire or pin.

The clinician can mark the midline location of first metatarsal 210 and a location of first MTP joint 230. In some examples, the clinician uses a marking source (e.g., a surgical marker pen) to indicate the midline location and the MTP joint. Additionally or alternatively, the clinician may percutaneously insert a wire into the MTP joint to identify the location of the MTP joint by the position of the wire extending out through the skin.

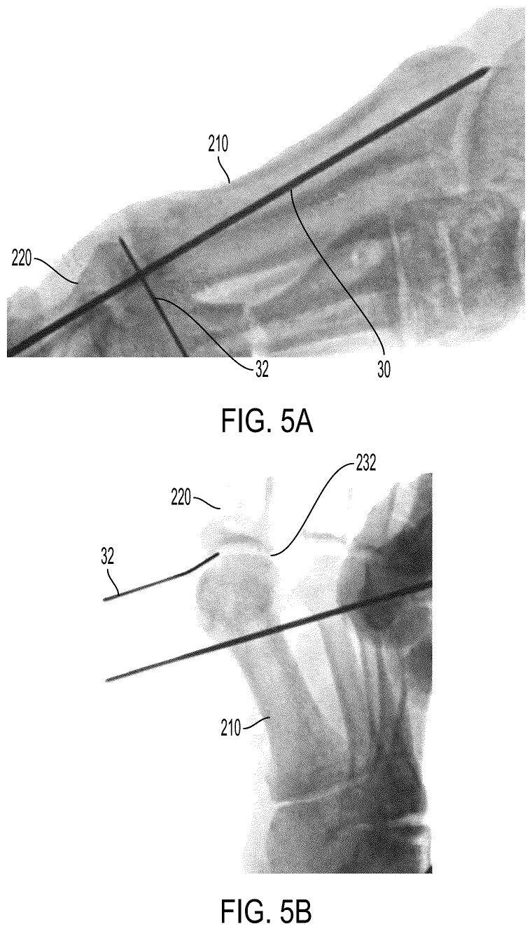

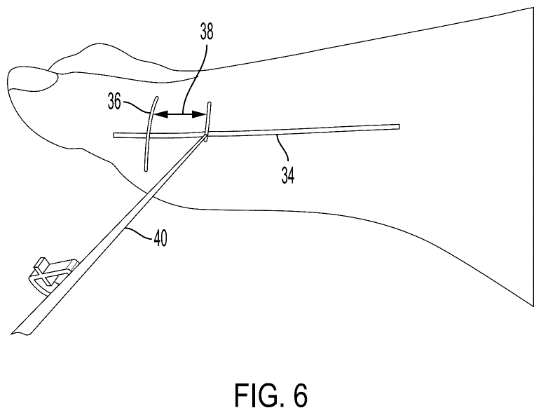

FIG. 5A is a medial-to-lateral view fluoroscopic image of an example portion of a foot showing a radiopaque K-wire 30 positioned along the midline of the foot to identify the midline location. FIG. 5A also illustrates a radiopaque wire 32 inserted into the first MTP joint 232 to mark the location of the joint. FIG. 5B is a dorsal-to-planar view fluoroscopic image of an example portion of the foot from FIG. 5A showing the positioning of radiopaque wire 32 inserted into first MTP joint 232. FIG. 6 shows a medial side of a foot with the midline of the first metatarsal marked along the skin by a midline marking line 34 and the MTP joint marked along the skin by a MTP marking line 36.

While first metatarsal 210 can be cut at any desired location along the length of the bone, in some examples, the location for cutting the bone is set relative to a joint (e.g., MTP joint 232; TMT joint 230). For example, the target location where first metatarsal 210 is to be cut and, correspondingly, the location where incision is to be made through the skin may be set based on the location of MTP joint 232. The clinician may identify a location offset along the midline length of first metatarsal 210 a distance 38 (FIG. 6) from MTP joint 232 (e.g., as indicated by a wire extending out of the joint and/or MTP marking line 36). Distance 38 may within a range from 10 mm to 40 mm, such as from 15 mm to 25 mm, or approximately 20 mm (plus or minus 10%).

After identifying a target cut location offset by distance 38 from MTP joint 232, the clinician can make an incision at the target location to access the underlying bone. In some examples, the clinician uses a ruler to measure the offset distance 38 and/or uses a marking instrument to mark the target location. In some examples, the clinician may position an incision guide at a target location, and the incision guide may have a predefined offset corresponding to distance 38 for indexing relative to MTP joint 232.

Before or after making an incision at a target location offset from MTP joint 232 by distance 38, the clinician may insert a K-wire 40 into first metatarsal 210. The K-wire can be inserted at the midline of first metatarsal 210 (midline in the dorsal-to-plantar direction) and can extend medially outwardly from a remainder of the foot. In either case, the clinician can make an incision substantially centered about the target cut location and/or K-wire 40 positioned at the target cut location. The clinician can make the incision by guiding a cutting instrument (e.g., scalpel) along the location wherein skin is to be cut with or without the aid of an incision guide define a guide surface (e.g., cut slot) for controlling the length of the skin incision. In practice, the incision may have a length within a range from 5 mm to 30 mm, such as from 10 mm to 25 mm, or from 15 mm to 20 mm. The incision may extend distal-to-proximally along the length of the metatarsal and/or dorsal-to-plantarly about the circumferential perimeter of the metatarsal.

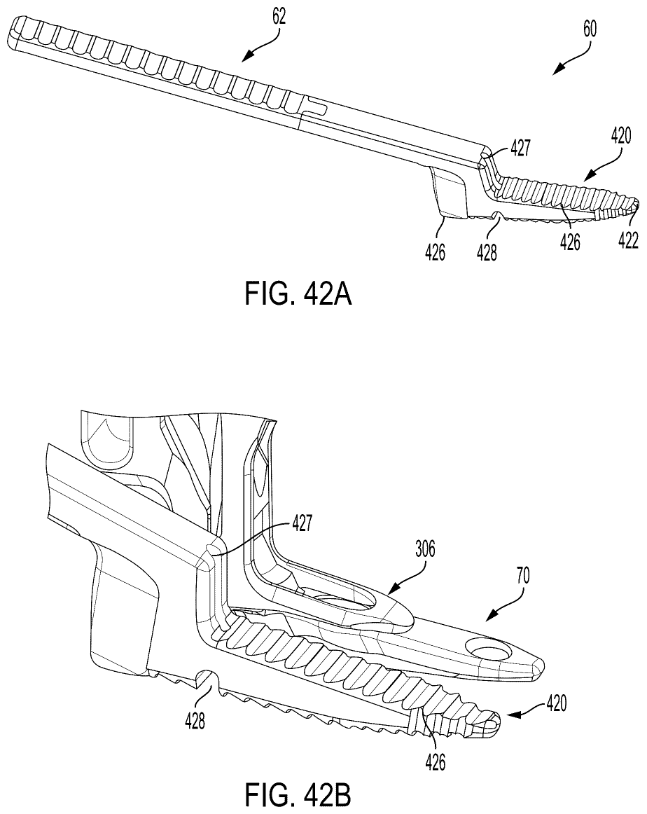

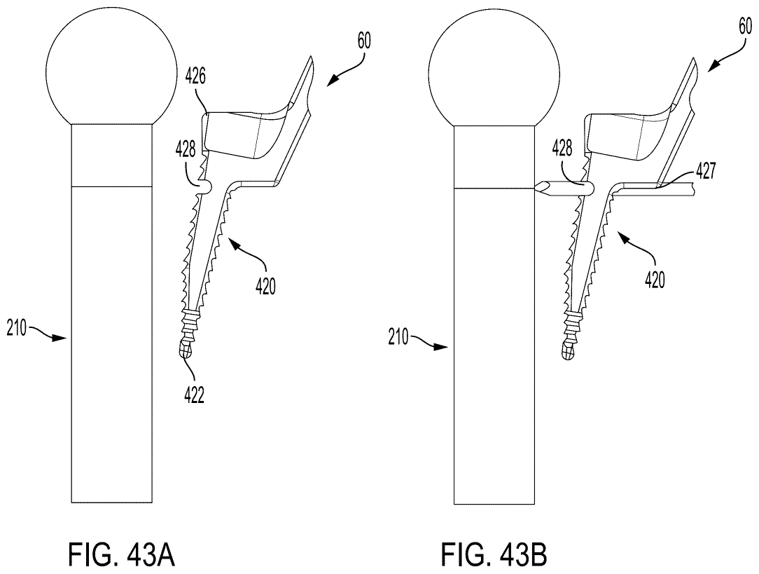

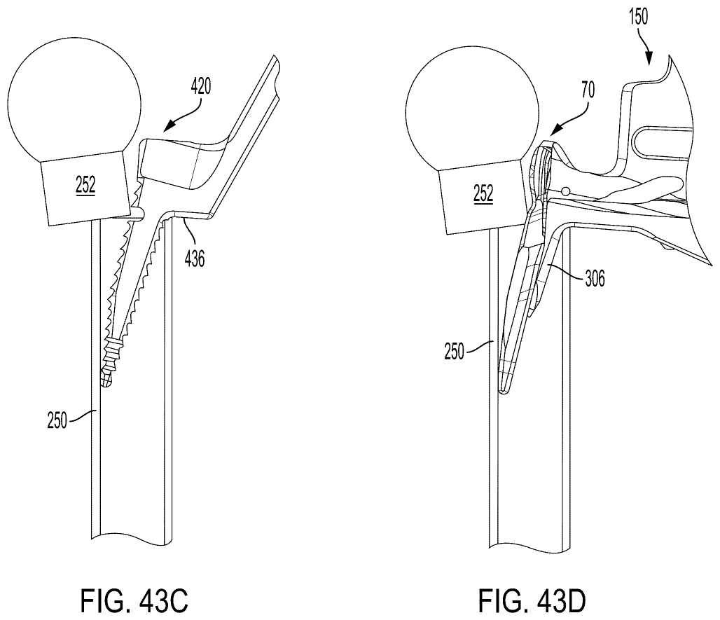

A clinician may identify a target location to cut first metatarsal 210 using other techniques, such as direct visualization without the aid of an instrument and/or through the use of guide defining a providing a measured offset distance from a target location. Another example instrument that can be used to define a target location to cut first metatarsal 210 into two portions is described with respect to FIGS. 42 and 43.

With first metatarsal 210 exposed through the incision in the skin, the example technique of FIG. 4 includes cutting the first metatarsal to form a distal metatarsal bone portion and a proximal metatarsal bone portion (step 12 on FIG. 4). For example, after creating an incision through the skin at a target location where first metatarsal 210 is to be cut, the clinician may insert a cutting instrument through the incision through the first metatarsal. In some examples, the clinician may cut first metatarsal 210 freehand by controlling the positioning and movement of the cutting instrument with their hand without the aid of a guide. In other examples, the clinician may cut first metatarsal 210 with the aid of a cutting guide (which can also be referred to as a bone preparation guide) having a guide surface positionable over first metatarsal 210 at the location with the bone is to be cut. When using a bone preparation guide, a cutting instrument can be inserted against a guide surface (e.g., between a slot define between two guide surfaces) to guide the cutting instrument for bone cutting.

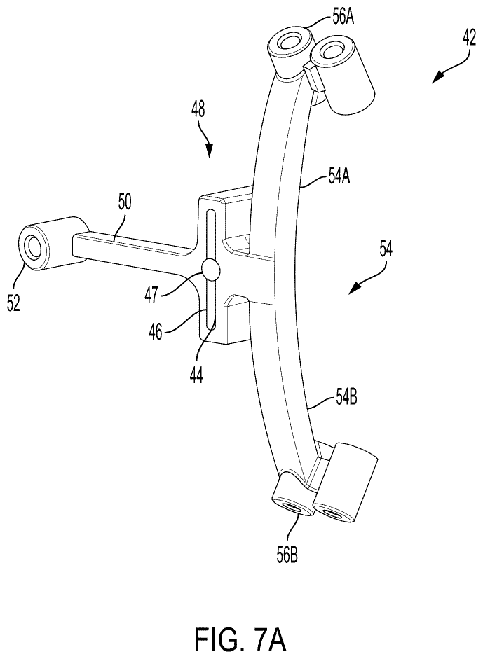

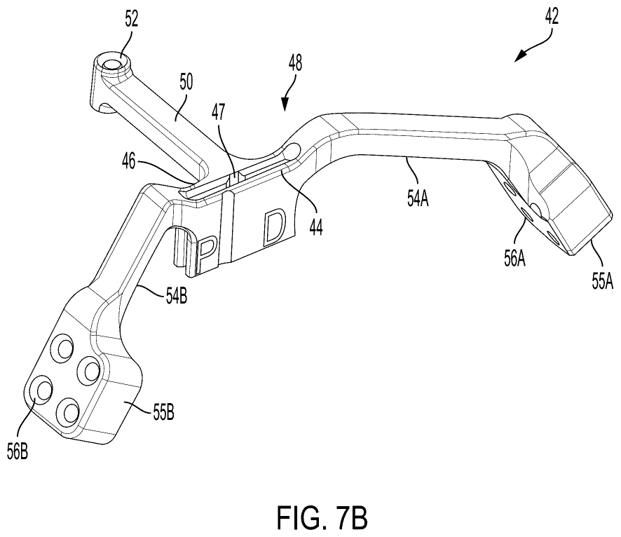

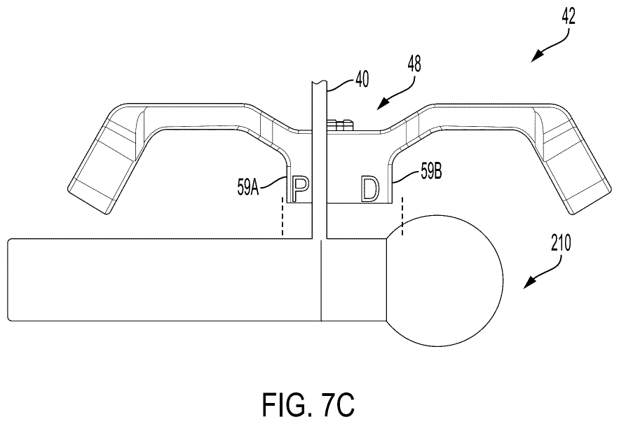

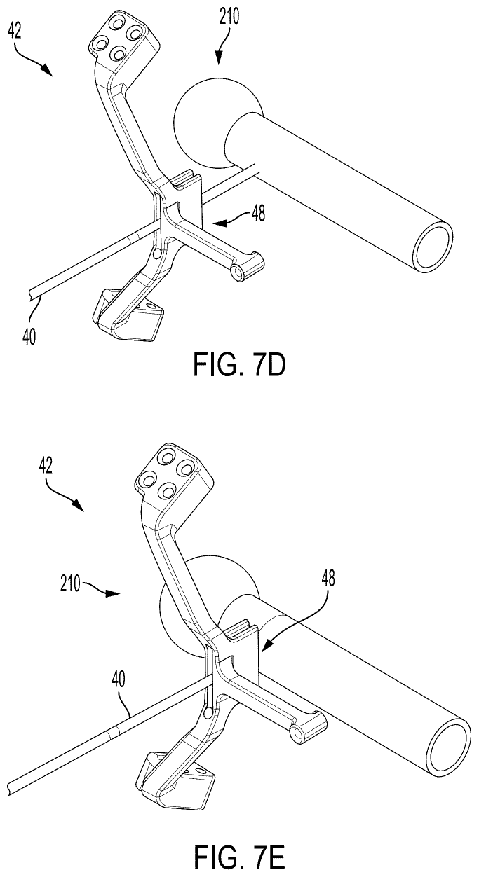

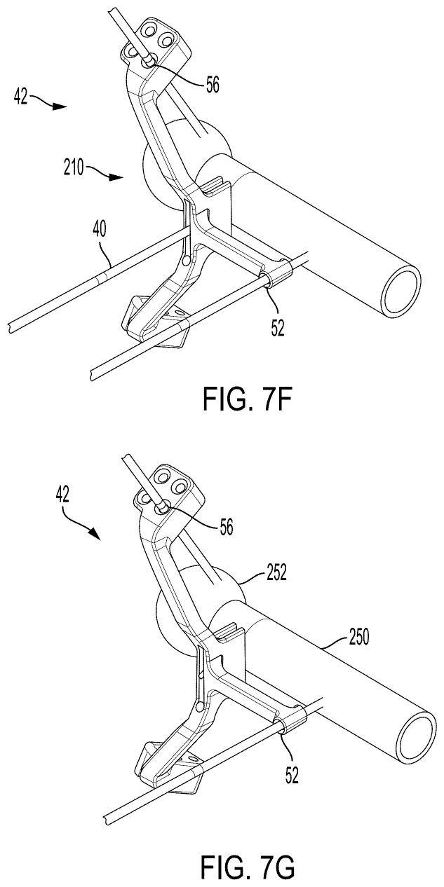

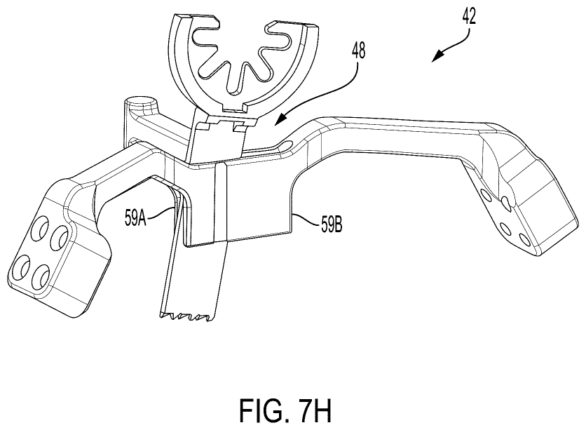

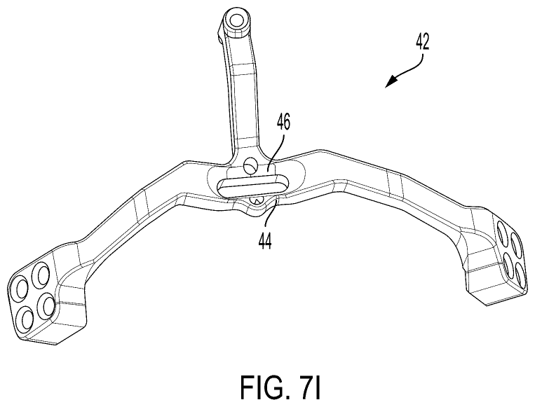

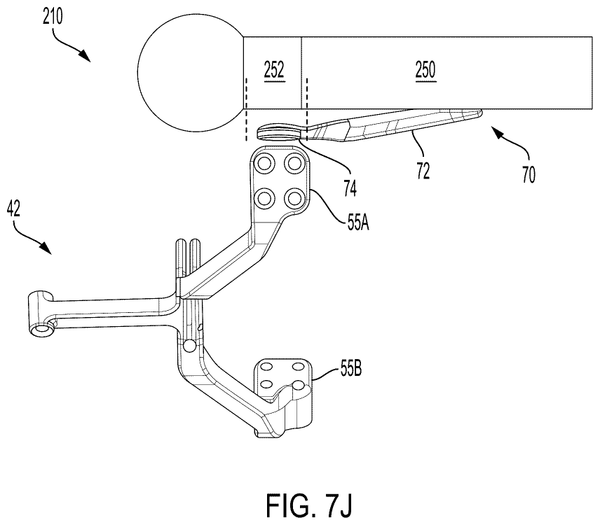

A variety of different bone preparation guides can be used to guide a cutting instrument. FIG. 7A illustrates one example bone preparation guide 42 that may be used as part of an osteotomy procedure. As shown, bone preparation guide 42 may define at least one guide surface 44 for guiding a cutting instrument. Guide surface 44 can be positioned over first metatarsal 210 at the location where the bone is to be cut. The clinician can guide the cutting instrument along the plane defined by guide surface 44 to control the location where first metatarsal 210 is cut. For example, the clinician may place the cutting instrument adjacent to and/or in contact with guide surface 44 and advance the cutting instrument into and through the first metatarsal 210 along the plane defined by the guide surface (e.g., by advancing the cutting instrument from the medial side of the metatarsal laterally through the lateral side of the metatarsal).

In some examples, bone preparation guide 42 defines a single guide surface 44 without opposed facing surface. In other examples, such as the example illustrated FIG. 7A, bone preparation guide 42 includes a second guide surface 46, which may be referred to as a facing guide surface, defining a plane parallel to the plane defined by guide surface 44. As a result, a cutting slot can be provided between the two guide surfaces 44, 46, with the two guide surfaces bounding the extent of the cutting slot. In use, the clinician can insert the cutting instrument through the cutting slot, with the cutting slot guiding the cutting instrument to define the trajectory of the cutting instrument for cutting first metatarsal 210.

The one or more guide surfaces 44, 46 of bone preparation guide 42 may be configured to cut first metatarsal 212 transversely in the transverse plane (e.g., in a plane parallel to the frontal plane of the bone). When so configured, the cut ends of the bone portions may extend at an approximately 90° angle relative to the longitudinal axis of first metatarsal 210. In other examples, the one or more guide surfaces may be skewed in the frontal plane and/or sagittal plane such that the cut ends of the resulting bone portions have end faces that are angled at a non-perpendicular angle with respect to the longitudinal axis of the first metatarsal 210.

In some configurations of bone preparation guide 42, the one or more guide surfaces 44, 46 of the bone preparation guide are sized relative to the expected size of the one or more bones to be prepared using the guide surface. For example, bone preparation guide 42 may include one or more guide surfaces 44, 46 having a length sized to be larger than or the same as the diameter of first metatarsal 210 at the location where the metatarsal is to be cut. In other examples, the one or more guide surfaces 44, 46 may have a length sized to be smaller than the diameter of first metatarsal 210 at the location where the metatarsal is to be cut. In these applications, the clinician may angle the end of the cutting instrument projecting beyond the guide surfaces to cut regions of bone not covered by the one or more guide surfaces.

In some examples, such as that illustrated in FIG. 7A, bone preparation guide 42 defines a pin opening 47 configured (sized and positioned) to receive K-wire 40 inserted into first metatarsal 210. For example, where K-wire 40 has a diameter within a range from 0.8 to 3.0 millimeters, such as from 0.8 mm to 2.0 mm (e.g., 1.6 mm), pin opening 47 may have a corresponding diameter sized to receive the K-wire therethrough. As illustrated, pin opening 47 is located substantially centered along the length of the one or more guide surfaces 44, 46, although in other examples may be positioned at other locations relative to the guide surfaces. Configuring bone preparation guide 42 with pin opening 47 may be useful to help position and orient the guide surfaces 44, 46 relative to the target cut location (e.g., by positioning the bone preparation over the K-wire).

Depending on the configuration of bone preparation guide 42, the clinician may position the one or more guide surfaces 44, 46 (e.g., cutting slot defined thereby) at the target location where first metatarsal 210 is to be cut and hold the bone preparation guide at the location while the cutting instrument is guided by the bone preparation guide to cut the first metatarsal. Additionally or alternatively, bone preparation guide 42 may be configured to be pinned to first metatarsal 210 to help stably position the bone preparation guide during subsequent use. Accordingly, bone preparation guide 42 can include one or fixation holes through which one or more corresponding pins can be inserted to pin the bone preparation guide to underlying bone.

In the example of FIG. 7A, bone preparation guide 42 is illustrated as having a body 48 defining first guide surface 44 and second guide surface 46. Bone preparation guide 42 may define at least one pin receiving hole for pinning the bone preparation guide to the first metatarsal, such as at least one pin receiving hole positionable proximally from where first metatarsal 210 is to be cut using the bone preparation guide and at least one pin receiving hole positioned distally from where first metatarsal 210 is to be cut using the bone preparation guide. When so configured, pins can be inserted on both sides of the cut to be made using the bone preparation guide, helping to stabilize the bone preparation guide while cutting the first metatarsal and after the first metatarsal has been cut into two different portions.

In the illustrated example, bone preparation guide 42 is illustrated as including a first arm 50 extending outwardly from body 48 to define a first pin receiving hole 52 at the end of the body. First arm 50 is illustrated as extending generally perpendicularly relative to the length of guide surfaces 44, 46. As a result, when bone preparation guide 42 is positioned over first metatarsal 210 with first and second guide surfaces 44, 46 defining guide planes across the cross section of first metatarsal 210, first arm 50 may extend parallel to the longitudinal length of the first metatarsal (in a distal to proximal direction).

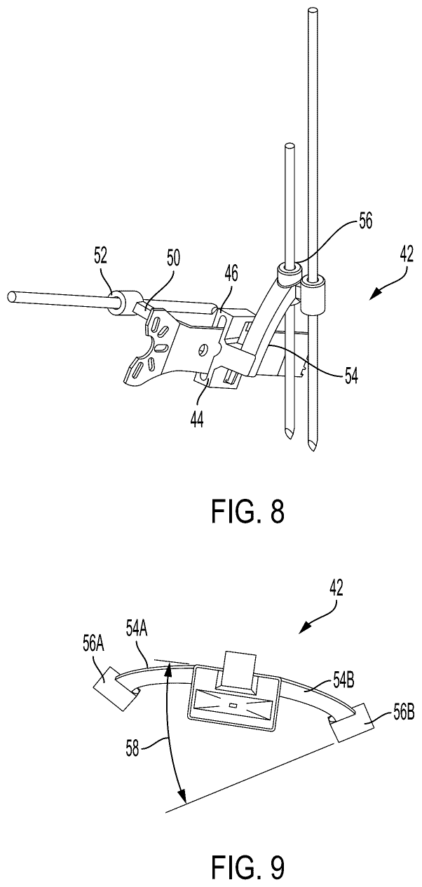

Bone preparation guide 42 is also illustrated as including a second arm 54 defining a second pin receiving hole 56, which is illustrated as including at least two pin receiving holes 56A and 56B. The second arm 54 may be configured to extend parallel to the longitudinal length of first metatarsal 210 (e.g., mirroring the arrangement of first arm 50). As illustrated, however, second arm 54 extends generally parallel to the plane defined by first guide surface and second guide surface 44, 46. For example, second arm 54 can define a radius of curvature extending outwardly from body 48 to position second pin receiving hole 56 at a location that is offset about the perimeter of first metatarsal 210 in the frontal plane from the first and second guide surfaces. For example, bone preparation guide 42 may include a second arm 54 having a first portion 54A configured to wrap dorsally upwardly from the medial side of the first metatarsal where the guide surfaces are to be positioned, thereby positioning pin receiving hole 56A over a dorsal-medial and/or dorsal side of the metatarsal. Bone preparation guide 42 may additionally or alternatively include a second arm 54 having a second portion 54B configured to wrap plantarly downwardly from the medial side of the first metatarsal where the guide surfaces are to be positioned, thereby positioning pin receiving hole 56B over a plantar-medial and/or plantar side of the metatarsal. FIG. 8 is a perspective view of another example configuration of bone preparation guide 42 in which the bone preparation guide is configured with a second arm portion configured to wrap in a single direction (e.g., plantarly or dorsally) about the metatarsal rather than two arm portions configured to wrap in both directions.

With further reference to FIG. 7A, when configured with one or more arms 54A, 54B that are configured to extend about first metatarsal 210, the one or more pin receiving holes 56A, 56B may extend at any desired angle. FIG. 9 is a side view of bone preparation guide 42 from FIG. 7A illustrating an example angular orientation of pin receiving hole 56 relative to a plane tangent to the outermost face of the bone preparation guide. As shown in this example, pin receiving hole 56 may be positioned on the end of second arm 54 and angled an angle 58 relative to a plane tangent to the outermost face of the bone preparation guide. In various examples, angle 58 may be within a range from 15° to 75°, such as from 20° to 45°, or from 25° to 35°. The pin receiving hole(s) at the opposite end of second arm 54 may be angled at the same or different angles as angle 58, albeit in opposite direction.

In some configurations, bone preparation guide 42 includes at least one pin receiving opening on either side of the target cut location to which parallel pins can be inserted. This can allow the bone preparation to be lifted off the parallel pins after cutting while leaving the parallel pins in place. In other configurations, one or more pin receiving openings on one side of the target cut location are angled relative to one or more pin receiving openings on the other side of the target cut location, e.g., such that pins inserted through the openings are angled relative to each other and the bone preparation guide cannot be lifted directly off the pins.

Bone preparation guide 42 can have a single pin receiving hole (which may also be referred to as a fixation hole) associated with each arm and/or side of the guide. Alternatively, bone preparation guide 42 may include multiple pin receiving holes associated with and/or defined by each side of the guide and/or arm extending from the guide. For example, as shown in FIG. 7A, first arm 50 is illustrated as defining a single pin receiving hole 52. By contrast, the first portion 54A of the second arm as illustrated as defining a pair of adjacent pin receiving holes, and the second portion 54B the second arm is illustrated as defining another pair of adjacent pin receiving holes. In general, any pin receiving hole may be implemented as a single pin receiving hole or an array of multiple adjacent pin receiving holes (e.g., two, three, or more) to provide flexibility and options for pinning the guide at different locations.

FIG. 7B is a perspective illustration of another example configuration of bone preparation guide 42, where like elements refer to like features discussed above with respect to the example of FIG. 7A. In the example of FIG. 7B, bone preparation guide 42 is illustrated as including a first arm 50 extending outwardly from body 48 to define a first pin receiving hole 52 at the end of the body. Bone preparation guide 42 is also illustrated as including a second arm 54 defining a second pin receiving hole 56, which is illustrated as including at least two pin receiving holes 56A and 56B. As illustrated, second arm 54 includes a first portion 54A configured to wrap dorsally upwardly from the medial side of the first metatarsal where the guide surfaces are to be positioned, thereby positioning pin receiving hole 56A over a dorsal-medial and/or dorsal side of the metatarsal. Second arm 54 is also illustrated as having a second portion 54B configured to wrap plantarly downwardly from the medial side of the first metatarsal where the guide surfaces are to be positioned, thereby positioning pin receiving hole 56B over a plantar-medial and/or plantar side of the metatarsal.

As illustrated, the first portion 54A of second arm 54 includes an array of multiple pin receiving hole 56A (e.g., offset medially-to-laterally and/or proximal-to-distally from each other when the bone preparation guide is oriented in a target cutting position). Second portion 54B of second arm 54 may also include an array of multiple pin receiving hole 56B (e.g., offset medially-to-laterally and/or proximal-to-distally from each other when the bone preparation guide is oriented in a target cutting position). In the specific example of FIG. 7B, first portion 54A of second arm 54 defines a first block 55A having an array of four pin receiving holes arranged in a generally square arrangement, and second portion 54B of second arm 54 defines a second block 55B having an array of four pin receiving holes arranged in a generally square arrangement. Bone preparation guide 42 may define a different number or arrangement of pin receiving holes.

In the illustrated example, bone preparation guide 42 also includes a groove or marking 57, which may be used for defining the length of the incision to be made through the skin of the patient prior to cutting the first metatarsal into multiple portions. During a surgical procedure, a clinician can use fluoroscopy and a guidewire over the skin of the patient and can mark a target cut location and/or centerline of the metatarsal shaft. The clinician can visualize and set the target cut location while visualizing under fluoroscopy. The clinician can then insert a wire into the first metatarsal at the target osteotomy location (e.g., directly under fluoroscopic visualization and/or at a location marked on the skin of the patient as determined during prior fluoroscopic visualization). With a wire inserted percutaneously through the skin of the patient, the clinician can determine the location and size of incision to be made through the skin of the patient encompassing the target cut location.

The clinician can align the groove or marking 57 indicated on bone preparation guide 42 with a wire inserted into the metatarsal and use body 48 of the bone preparation to define the length and position of the incision to be made through the skin of the patient. For example, FIG. 7C illustrates bone preparation guide 42 positioned adjacent first metatarsal 210 (e.g., overlying the skin of the patient covering the metatarsal) with wire 40 aligned with a positioning indicator 57 defined by the bone preparation which, is illustrated as being a groove that wire 40 can fit into. Body 48 defines a proximal edge 59A in a distal edge 59B that can be used to set the proximal-most extent and distal-most extent of an incision to be made through the skin of the patient. With some implant systems, the incision may be longer in one direction than another relative to the target osteotomy location. For example, the incision through the skin of the patient may be longer distally to the osteotomy location then proximal to the osteotomy location to provide additional surgical access space for accessing the cut faces of the bone portions and/or applying screws through a plate portion of an intramedullary implant. As shown in the illustrated example, positioning indicator 57 may be asymmetrically located more proximal than distal to define an incision length and location that extends longer distally to an osteotomy location then proximally from the osteotomy location. The clinician can then proceed to make the incision through the skin of the patient (e.g., on the medial side of first metatarsal 210) following the length and location defined by body 48 as oriented relative to wire 40 using positioning indicator 57.

After making the incision through the skin of the patient, the clinician can insert a bone preparation guide 42 over wire 40 and into the incision (e.g., until the cut guide abuts first metatarsal 210). In some examples, the clinician inserts bone preparation guide 42 down over pin 40 such that the length of body 48 (e.g., bone preparation slot) is parallel to the incision as the body is advanced through the incision. Thereafter, the clinician may rotate bone preparation guide 42 (e.g., 90°) such that the slot and/or guide surfaces defined by bone preparation guide 42 are perpendicular to the length of the incision through the skin and/or first metatarsal 210. Inserting the bone preparation guide 42 parallel through the incision and then rotating can function to retract tissue and allow the guide to sit better against the bone. FIGS. 7D and 7E illustrate bone preparation guide 42 being advanced over wire 40 (perpendicular to the length of the incision and seated against first metatarsal 210. Before inserting bone preparation guide 42 through the incision, soft tissue preparation may be performed. Such soft tissue preparation can include stripping of the medial cortex of first metatarsal 210 proximally and/or distally of the osteotomy location, e.g., to allow the plate portion of the intramedullary implant to sit flush against the face the bone and/or to remove inhibiting tissue that is difficult access.

FIG. 7F illustrates bone preparation guide 42 being secured to first metatarsal 210 with a fixation wire inserted into the metatarsal proximally of the target osteotomy location through pin receiving opening 52 and also distally of the target osteotomy location through pin receiving opening 56. Pin receiving opening 52 may be aligned with the central axis of first metatarsal 210 (e.g., using an external mark on the skin made when initially laying out the osteotomy) to ensure the resulting cut through the bone is perpendicular to the longitudinal axis of the metatarsal shaft. This can help prevent off axis movements introduced by a non-perpendicular osteotomy, helping to ensure that the cut end faces of the resulting bone portions are substantially perpendicular to the longitudinal axis of the bone (plus or minus 10 degrees or less, such as 5 degrees or less, or 3 degrees or less from absolutely perpendicular).

Once secured proximally and distally of the target osteotomy location, wire 40 introduced into metatarsal 210 at the target osteotomy location can be removed, as shown in FIG. 7G, and a bone preparation instrument guided along a guide surface (e.g., through a slot bounded by two guide surfaces) to divide the first metatarsal into a proximal metatarsal bone portion 250 in a distal metatarsal bone portion 252. The wire inserted through pin receiving opening 52 can then be removed and bone preparation guide 42 slid off the wire extending through pin receiving opening 56. This wire inserted into distal metatarsal bone portion 252 can be used subsequent during a realignment process for controlling repositioning of the distal metatarsal bone portion (e.g., in the frontal plane and/or sagittal plane).

Bone preparation guide 42 can utilize a variety of different features and configurations. FIG. 7H is an illustration of an example configuration of bone preparation guide 42 that includes a cutout extending through proximal edge 59A of body 48 and/or through distal edge 59B of body 48. Configuring bone preparation guide 42 with a sidewall cutout on one or both sides delimiting the extent of the cutting slot can be useful to allow a bone preparation instrument to be angled beyond the terminal extent of the cutting slot on one or both sides. When performing the osteotomy, a clinician can advance a bone preparation instrument through the cutting slot an angle the terminal end of the bone preparation instrument beyond the terminal end or ends delimited by the cutting slot to increase the extent of the cut. This can enlarge the length that the bone preparation instrument can sweep through and cut bone while minimizing the size of body 48 to be inserted through an incision in the skin of the patient.

As another example configuration, bone preparation guide 42 can define one or more guide surfaces configured to guide a burr bone preparation instrument rather than a saw blade. FIG. 7I is a perspective illustration of an example configuration of bone preparation guide 42 configured for use with a burr. As shown in this example, the bone preparation guide defines a first guide surface 44 and a second guide surface 46, with the two guide surfaces bounding the extent of a cutting slot there between. To allow the use of a burr bone preparation instrument, the cutting slot may be sized wider in shorter than a comparative cutting slot configured for a saw blade.

As another example configuration feature for bone preparation guide 42, one or both of the blocks 55A, 55B defining one or more pin receiving holes extending therethrough may have a size corresponding to a size of a portion of an intramedullary implant 70 to be installed during the surgical procedure. For example, FIG. 7J is a perspective illustration of an example configuration of bone preparation guide 42 showing first block 55A of the bone preparation having a size (e.g., width, length) that is substantially the same as the length of a plate portion 74 of an intramedullary implant 70. The clinician can position first block 55A relative to the head of first metatarsal 210 (optionally under fluoroscopy) to determine where the osteotomy location should be set. Plate portion 74 of intramedullary implant 70 may be positioned against a side of distal metatarsal bone portion 252 abutting and/or offset from the distal metatarsal head. Accordingly, block 55A can be used as a visualization aid to show the approximate positioning where plate portion 74 of intramedullary implant 70 will be located following the osteotomy, thereby allowing the clinician to identify the osteotomy location at the proximal end of plate portion 74.

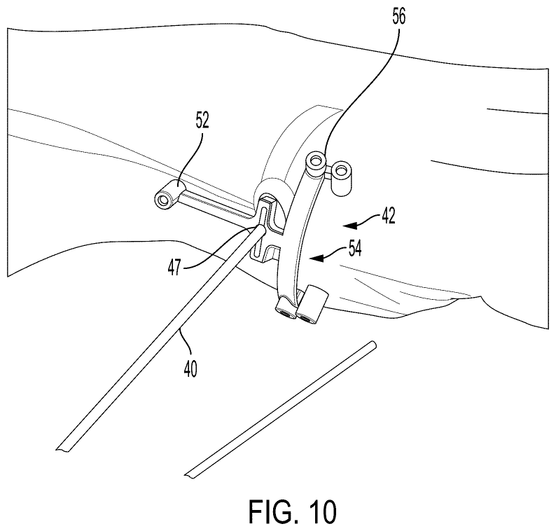

With further reference to the example bone preparation guide 42 of FIG. 7A, a clinician may position bone preparation guide 42 relative to a target location at which first metatarsal 210 is to be cut. For example, the clinician can align opening 47 of the bone preparation with K-wire 40 and advance the bone preparation down over the K-wire. FIG. 10 illustrates bone preparation guide 42 installed over K-wire 40 on an example foot of a patient with K-wire 40 extending out through opening 47 of the bone preparation guide.

A clinician can insert a first pin through a pin receiving opening 52, 54 on one side of the target cut location and insert the second pin through a pin receiving opening 52, 54 on the other side of the target cut location. The clinician may additionally or alternatively insert additional pins on one or both sides of the cut guide. Each pin inserted through a corresponding pin receiving opening 52, 54 may or may not be inserted percutaneously through the skin of the patient, e.g., which can minimize the length of the incision needed through the skin of the patient.

With bone preparation guide 42 suitably positioned and/or pinned over first metatarsal 210, the clinician can remove K-wire 40 from the opening 47 to expose the entire length of the opening for guiding a cutting instrument there through. The clinician can then guide a cutting instrument using the slot defined between the first and second guide surfaces 44, 46 to advance the cutting instrument through the first metatarsal 210 to cut the first metatarsal into two separate portions.

Example cutting instruments that can be used to cut first metatarsal 210 (which may also be referred to as tissue removing instruments) include, but are not limited to, a saw blade, a rotary bur, a rongeur, a reamer, an osteotome, a curette, and the like. In some examples, the clinician may use one cutting instrument (e.g., saw blade, rotary bur) to transect first metatarsal 210 into two portions and then further prepared the cut end faces of the two bone portions, e.g., by fenestrating, morselizing, and/or otherwise generating bleeding bone faces to promote fusion.

With further reference to FIG. 4, the example technique may include creating a pocket in one or both of the proximal metatarsal bone portion and the distal metatarsal bone portion formed by cutting first metatarsal 210 (step 14 in FIG. 4). For example, when preparing to fixate a moved position of the distal metatarsal bone portion relative to the proximal metatarsal bone portion using an intramedullary implant, a pocket may be formed in the bone portion configured to receive the stem of the intramedullary implant. The pocket can create an opening sized and oriented to receive and guide the stem of the intramedullary implant during installation. If using other fixation devices that do not include a stem or intramedullary implant, the clinician may forgo the step of creating a pocket in the metatarsal bone portion.

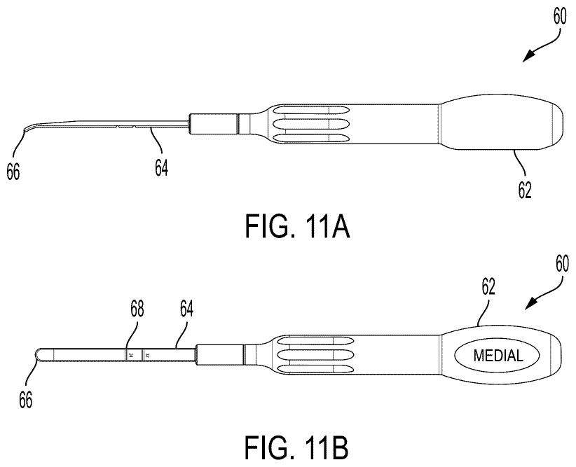

FIGS. 11A and 11B are side and top views, respectively, of an example broach instrument 60 that can be used to form a pocket in the end of a metatarsal bone portion to prepare the metatarsal bone portion to receive the stem of the intramedullary implant. In this example, broach instrument 60 includes a handle 62 graspable by a user to manipulate the broach instrument and a shaft 64 extending outwardly from the handle. Shaft 64 can have a size and shape corresponding to the size and shape of the stem of the intramedullary implant to be inserted into the pocket formed using the broach. The distal end 66 of shaft 64 may be straight or, as illustrated, curved to help guide insertion of the instrument into the end of the metatarsal bone portion. For example, shaft 64 may define a radius of curvature extending partially or fully along the length of the shaft. The curvature may be useful to help form a pocket extending at least partially across the diameter of the metatarsal.

One or more depth stop markings 68 may be indicated on shaft 64 of broach instrument 60. In use, a clinician can insert broach instrument 60 into the metatarsal bone portion until the instrument has been advanced to a desired depth corresponding to a depth stop markings 68. The desired depth stop marking 68 may be a length corresponding to the length of the intramedullary implant stem to be inserted into the pocket.

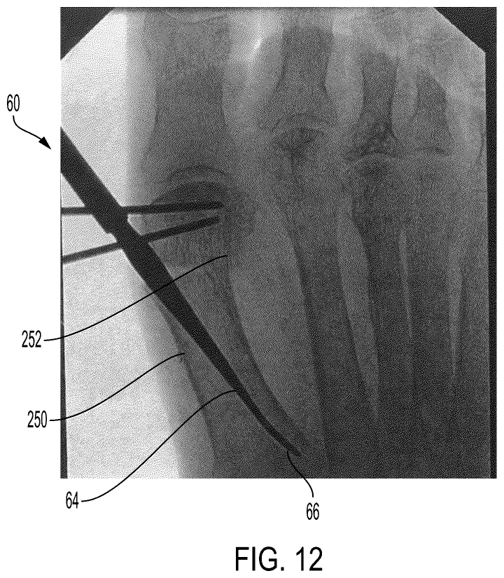

FIG. 12 is a fluoroscopic image showing broach instrument 60 inserted into a proximal metatarsal bone portion 250. As shown in this example, broach instrument 60 is inserted from the distal toward the proximal direction along the length of the first metatarsal. Distal metatarsal bone portion 252 formed by cutting the first metatarsal is offset laterally relative to the cut end face of the proximal metatarsal bone portion 250 formed by cutting the first metatarsal. In the illustrated example, shaft 64 of broach instrument 60 is advanced from the distal medial side of proximal metatarsal bone portion to a proximal lateral side of the proximal metatarsal bone portion. As a result, the pocket formed by broach instrument 60 extends both partially along the length of proximal metatarsal bone portion 250 as well as across the diameter of the proximal metatarsal bone portion. In other configurations, broach instrument 60 may be inserted at other angles relative to the metatarsal bone portion, e.g., such as axially along the length of the metatarsal bone portion in the medullary canal without substantially deviating medially or laterally.

While FIG. 12 illustrates an example configuration in which broach instrument 60 is inserted into proximal metatarsal bone portion 250 to subsequently insert an intramedullary implant into the proximal metatarsal bone portion, in alternative implementations, broach instrument 60 may additionally or alternatively be inserted into distal metatarsal bone portion 252. In these applications, the intramedullary implant stem may be inserted into the distal metatarsal bone portion 252 and pocket formed therein by the broach instrument. While FIGS. 11 and 12 illustrate one example broach instrument 60, other instruments capable of forming a pocket through the end face of a cut metatarsal bone portion can be used, the disclosure is not limited in this respect.

For example, as will be described with respect to FIGS. 42 and 43, a procedure may utilize a trialing instrument that has a trialing implant sized and shaped to correspond to the definitive implant to be used during the procedure. The trialing implant can be inserted into the intramedullary canal of a metatarsal bone portion to form a pocket mirroring the dimensions of the intramedullary implant stem for the definitive implant.

Independent of how an implant pocket is created in a bone portion, the example technique of FIG. 4 includes inserting an intramedullary implant stem into the pocket formed in the metatarsal bone portion (step 16 in FIG. 4). For example, the clinician can remove broach instrument 60 from the proximal metatarsal bone portion 250 and insert the stem of an intramedullary implant into the pocket formed by the broach instrument. In other examples, the clinician may insert the intramedullary implant into the metatarsal bone portion without first forming a pocket in the metatarsal bone portion using a separate instrument.

A variety of different fixation devices including intramedullary stem portions can be used as during an osteotomy procedure according to the disclosure. In some implementations, an intramedullary implant fixation device includes a stem portion insertable into the intramedullary canal of one metatarsal bone portion and in a plate portion connected to the stem portion that is positionable against an exterior surface of another metatarsal bone portion. For example, the intramedullary implant may include an intramedullary stem insertable into proximal metatarsal bone portion 250 and an integral plate portion positionable against an external side (e.g., medial side) a distal metatarsal bone portion 252 (e.g., with the implant portion and plate portion forming a unitary body). The plate portion of the intramedullary implant can include one or more fixation holes for receiving corresponding fixation devices therethrough to attach the plate portion to the distal metatarsal bone portion.

It should be appreciated that reference to an intramedullary device having a stem portion does not require that the stem have any particular shape or dimensions unless otherwise specified. In some examples, the intramedullary stem portion of the intramedullary implant has a length (e.g., in the distal to proximal direction when inserted into a bone portion) greater than a width (e.g., in the medial to lateral direction when inserted into a bone portion) and thickness (e.g., in the dorsal to plantar direction when inserted into a bone portion).

In some examples, the intramedullary stem portion of the intramedullary implant is sized to be press-fit into the intramedullary canal of the bone portion, e.g., such that the clinician cannot substantially or at all translate or shift the intramedullary stem portion of the intramedullary implant in the intramedullary canal once inserted. In these examples, the width of the intramedullary stem portion of the intramedullary implant may be sized relative to the intramedullary canal to have a size effective to position the intramedullary stem portion of the intramedullary implant extending across the entire intramedullary canal (e.g., with the stem in contact with a cortical wall of the bone portion on one widthwise side of the stem and also in contact with a cortical wall of the bone portion on an opposite widthwise side of the stem).

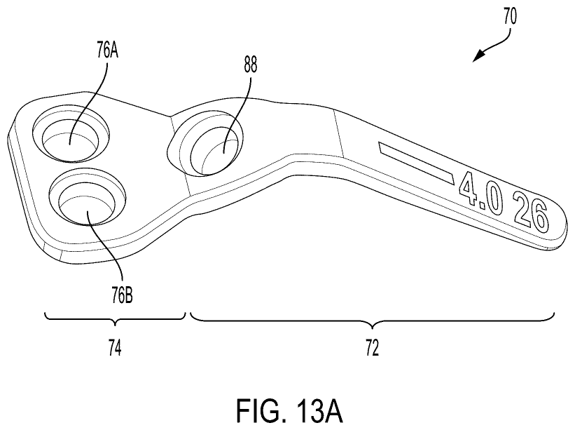

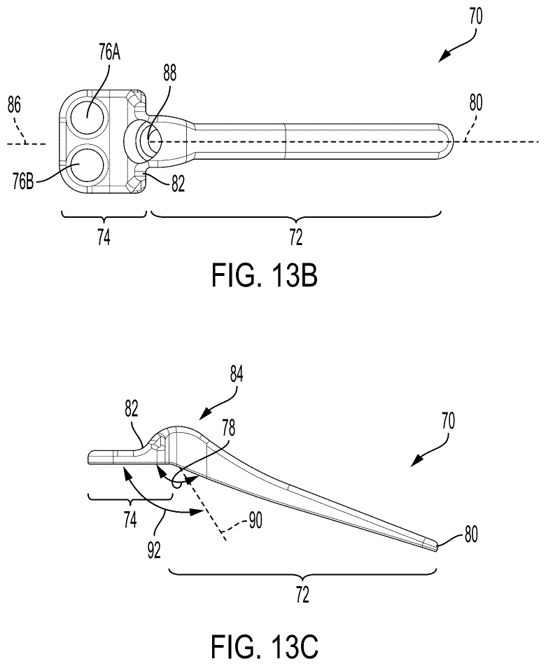

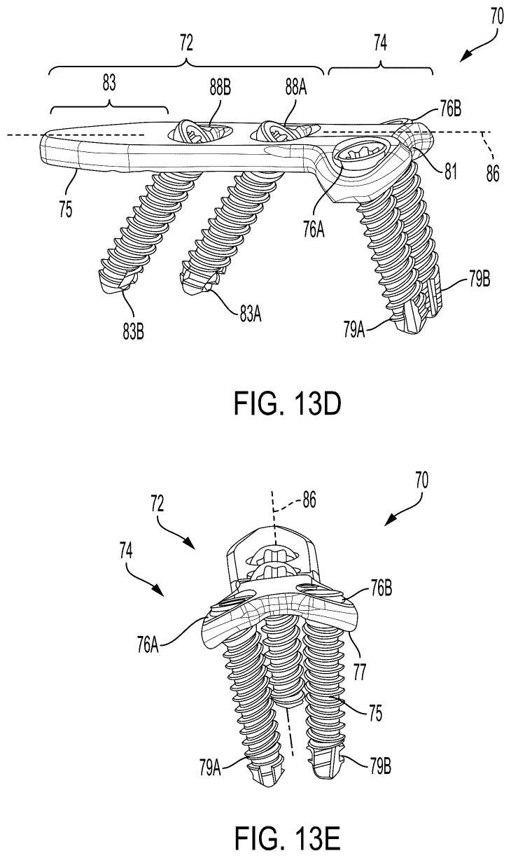

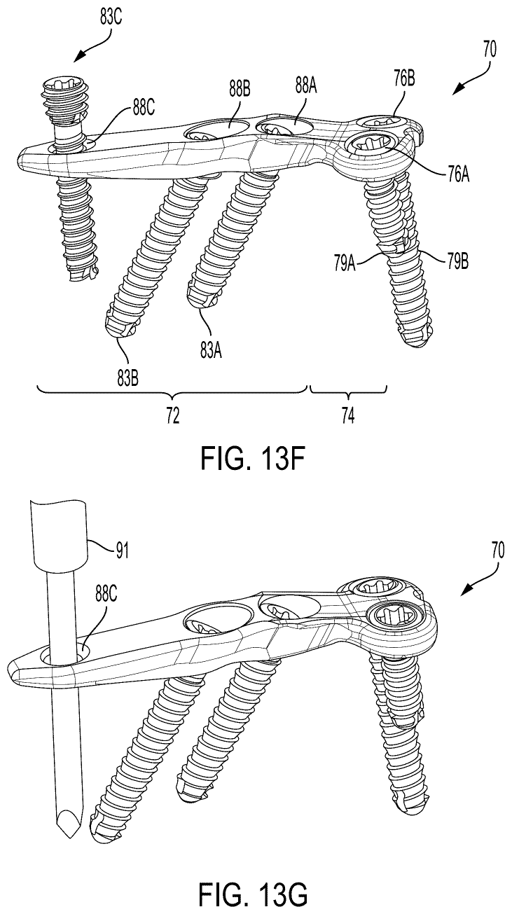

In other examples, including some implementations of the examples of FIG. 13 to be discussed, the intramedullary stem portion of the intramedullary implant may be sized smaller than the intramedullary canal into which the stem is inserted. For example, the width of the intramedullary stem portion of the intramedullary implant may be sized smaller than the diameter of the intramedullary canal of the bone portion into which the stem is to be inserted (e.g., such that there is an offset from the cortical wall of the bone portion into which the stem is inserted on one or both widthwise sides of the stem). As a result of the configuration, in some examples, the clinician may translate or shift the intramedullary stem portion of the intramedullary implant in the intramedullary canal once inserted. The clinician can shift the intramedullary stem portion in the intramedullary canal by shifting the distal metatarsal bone portion relative to the proximal metatarsal bone portion while the intramedullary stem remains in a substantially stationary location and/or by shifting the intramedullary stem relative to the intramedullary canal while the bone portion remains substantially stationary. In either case, under sizing the intramedullary stem portion of the intramedullary implant relative to the intramedullary canal can allow the clinician to rotate, translate, and/or otherwise move the intramedullary stem portion relative to the intramedullary canal once inserted.

FIGS. 13A-13C (collectively referred to as “FIG. 13”) are different views of an example intramedullary implant 70 according to the disclosure. FIG. 13A is perspective of the example intramedullary implant 70. FIG. 13B is a top view of the example intramedullary implant 70. FIG. 13C is a side view of the example intramedullary implant 70. As shown in this example, intramedullary implant 70 includes a body defining an intramedullary stem portion 72 and a plate 74. Intramedullary stem portion 72 can extend from plate 74 to form a unitary structure. Intramedullary stem portion 72 can be configured (sized, shape) for insertion into the medullary canal of a metatarsal bone portion. Plate 74 can define one or more fixation apertures 76 which, in the illustrated example is shown as two fixation apertures 76A and 76B. Fixation apertures 76 can receive fixation elements therethrough, such as screws, to secure the plate to an underlying bone portion.

In the illustrated arrangement, intramedullary stem portion 72 is illustrated as extending at an angle 78 relative to the surface defined by plate 74. When so configured, plate 74 can be positioned against an exterior surface of one metatarsal bone portion (e.g. distal metatarsal bone portion 252) and intramedullary stem portion 72 can extend lengthwise into an opposed metatarsal bone portion (e.g., proximal metatarsal bone portion 250) and cross from a medial side of the opposed metatarsal bone portion to the lateral side of the opposed metatarsal bone portion at the angle set by angle 78. Configuring intramedullary stem portion 72 to extend angularly away from plate 74 may be useful to position the intramedullary stem crossing across the metatarsal bone portion into which it is inserted. This can help retain the intramedullary stem in the metatarsal bone portion into which it is inserted, to resist pull up and promote fusion. In addition, this angular offset can help align the relative orientation of distal metatarsal bone portion 252 to proximal metatarsal bone portion 250.

In some examples, angle 78 between intramedullary stem portion 72 and plate 74 is within a range from 100° to 175°, such as from 120° to 160°. In some examples, angle 78 between intramedullary stem portion 72 and plate 74 is within a range from 145° to 175°, such as from 150° to 170°, from 165° to 175°, from 155° to 165°, or from 145° to 155°. Angle 78 can be measured between a lengthwise axis bisecting stem 72 and a lengthwise axis bisecting plate 74. The overall length of intramedullary implant 70, including intramedullary stem portion 72 of the intramedullary implant may vary. In some examples, intramedullary stem has a length extending from a first end 80 to a second end 82 within a range from 20 mm to 50 millimeters, such as from 25 mm to 40 mm. The cross-sectional size of intramedullary stem portion 72 may vary and, in some examples, is within a range from 3 mm to 10 mm, such as from 4 mm to 6 mm. While intramedullary stem portion 72 of intramedullary implant 70 is illustrated as having a generally rectangular cross-sectional profile in the example of FIG. 13, the intramedullary stem can have other polygonal and/or arcuate (e.g., circular) cross-sectional profiles. In some examples, intramedullary stem portion 72 includes one or more grooves, ribs, and/or other surface features that function to help retain the intramedullary stem into the bone portion in which the stem is inserted.

The thickness of intramedullary implant 70 may be the same across the entire length of the intramedullary implant, or the intramedullary implant may have one or regions that have a thickness differing from one or more other regions of the implant. For example, in the illustrated arrangement, intramedullary stem portion 72 defines an enlarged region 84 adjacent the second end 82 of the intramedullary stem. Enlarged region 84 may be a region of increased thickness relative to a remainder of the intramedullary implant, e.g., to provide structural strength and rigidity to the implant. When so configured, intramedullary stem portion 72 may have a thickness that tapers from second end 82 toward first end 80. Plate 74 of the intramedullary implant may also define a thickness less than that of the enlarged region 84.

In the example of FIG. 13, plate 74 of intramedullary implant 70 defines at least one fixation aperture 76 configured to receive a fixation screw therethrough. In the illustrated arrangement, plate 74 of intramedullary implant 70 has two fixation apertures 76A and 76B positioned side-by-side with each other, with the two fixation apertures being positioned on opposite sides of an axis 86 bisecting the intramedullary implant lengthwise. Upon inserting intramedullary stem portion 72 into the metatarsal bone portion such as proximal metatarsal bone portion 250, first fixation aperture 76A may be positioned comparatively dorsally to the second fixation aperture 76B, and the second fixation aperture 76B positioned comparatively proximately to the first fixation aperture.

In different examples, intramedullary implant 70 can have a different number or arrangement of fixation apertures on the plate portion 74 of the implant. For example, plate 74 may have only a single fixation aperture 76 (e.g., substantially centered on the axis 86 bisecting the intramedullary implant lengthwise) or have three or more fixation apertures. As another example, plate 74 may have two or more fixation apertures 76 positioned in a different arrangement than that illustrated in FIG. 13. For example, the two or more fixation apertures 76 may be positioned side-by-side lengthwise along the length of intramedullary implant 70 (e.g., each substantially centered on the axis 86 bisecting the intramedullary implant lengthwise) rather than being offset from the centerline.

Intramedullary stem portion 72 may or may not include one or more fixation apertures 88 configured to receive one or fixation members (e.g., screws) therethrough. For instance, in some examples, intramedullary stem portion 72 may be inserted into proximal metatarsal bone portion 250 with frictional resistance between the intramedullary stem and bone portion retaining the intramedullary stem in the bone portion. Additional fixation screws may not be used to secure the intramedullary stem to the proximal metatarsal bone portion 250. In other examples, supplemental fixation may be used to help retain the intramedullary stem portion 72 in the proximal metatarsal bone portion 250. In these examples, one or more additional fixation apertures 88 may be positioned at one or more locations along the length of intramedullary stem 72. For example, a fixation aperture 88 may be positioned adjacent the second end 82 of intramedullary stem 72.

After inserting intramedullary stem portion 72 into proximal metatarsal bone portion 250, a screw can be inserted into fixation aperture 88 and into the underlying bone. Fixation aperture 88 may be positioned at the cut end face of proximal metatarsal bone portion 250. As a result, when a screw is inserted into fixation aperture 88, the screw may enter the fixation aperture at the cut end face of proximal metatarsal bone portion 250 and be advanced into and/or through the proximal metatarsal bone portion. For example, a screw may be inserted into fixation aperture 88 at the cut end face of proximal metatarsal bone portion 250 and advanced through a lateral cortical wall of the proximal metatarsal bone portion (e.g., such that the distal end of the screw is positioned in the intermetatarsal space between the adjacent second metatarsal and the lateral side of the proximal metatarsal bone portion 250 following fixation).

Fixation apertures 88 can be positioned to define a variety of different screw trajectories for the screw inserted therein. In some examples, fixation apertures 88 defines a screw trajectory 90 that extends at an angle 92 relative to the surface defined by plate 74. In some examples, angle 92 is within a range from 110° to 175°, such as from 125° to 160°.

FIGS. 13D and 13E are a perspective view and a back view, respectively, of another example configuration of intramedullary implant 70. As shown in this example, intramedullary implant 70 again includes a body defining an intramedullary stem portion 72 and a plate 74. Intramedullary stem portion 72 is illustrated as extending from plate 74 and defining a unitary structure therewith. Intramedullary stem portion 72 and intramedullary plate portion 74 are illustrated as being co-linear with each other along axis 86 bisecting the intramedullary implant lengthwise such that a bottom surface 75 of intramedullary stem portion 72 at a location along axis 86 is in the same plane as the bottom surface 75 of plate 74 at a location along the axis.

As illustrated in FIG. 13E, the bottom surface of the distal end of plate 74 may define a radius of curvature configured to conform to a curvature the metatarsal head (e.g., diaphysis of the head) and/or the metatarsal shaft against which plate 74 is to be placed in use. When configured with a radius of curvature, the bottom surface of at least plate 74 may form a concave shape such that the bottom surface 77 of the plate at the widthwise-side edges of the plate is offset relative to the bottom surface 75 of the plate at the lengthwise center of the plate.

In the illustrated arrangement of FIGS. 13D and 13E, plate 74 of intramedullary implant 70 has at least one fixation aperture, which is illustrated as two fixation apertures 76A and 76B positioned side-by-side with each other, with the two fixation apertures being positioned on opposite sides of an axis 86 bisecting the intramedullary implant lengthwise. Fixation apertures 76A and 76B can be oriented relative to each other in a number of different ways, e.g., such that screws 79A and 79B inserted through the respective fixation apertures are parallel to each other, converge toward each other, or diverge away from each other. As illustrated, apertures 76A and 76B are oriented relative to each other such that screws 79A and 79B inserted therethrough converge towards each other. As a result, the distance separating the ends of the screws located farthest away from plate 74 is less than the distance separating the heads of the screws engaged plate 74.

In some configurations, the distal end of the plate 74 defines a recess 81. For example, plate 74 may include at least two lobes defining fixation apertures 76A and 76B. The distalmost extent of the lobes along the lengthwise direction of intramedullary implant 70 may extend beyond the distalmost extent of plate 74 at the widthwise center of the plate (e.g., at the location bisected by axis 86) to define recess 81. Recess 81 may provide a feature or space that can receive a portion of an intramedullary implant insertion and positioning instrument (discussed in greater detail below), although the instrument to hold intramedullary implant 70 for insertion into a bone portion.

Intramedullary stem portion 72 of intramedullary implant 70 in the example of FIGS. 13D and 13E includes at least one fixation aperture, which is illustrated as at least two fixation apertures 88A, 88B configured to receive at least two screws 83A and 83B therethrough. Fixation apertures 88A and 88B can be oriented relative to each other in a number of different ways, e.g., such that screws 83A and 83B inserted through the respective fixation apertures are parallel to each other, converge toward each other, or diverge away from each other. In the illustrated arrangement, fixation apertures 88A and 88B are oriented relative to each other such that screws 83A and 83B extend parallel to each other. While the screws can extend at a variety of different angles relative to intramedullary stem 72, in some examples, the screws are angled at an acute angle relative to the bottom surface 75 of the stem. This can define screw trajectories that orients screw 83A and 83B diverging away from distal metatarsal bone portion 252 (such that the end of the screw is positioned more proximally of distal metatarsal bone portion 252 than the head of the screw engaging the implant), when intramedullary stem portion 72 is inserted into proximal metatarsal bone portion 250 and plate 74 is attached to distal metatarsal bone portion 252.

Fixation apertures 88A, 88B may be positioned at various locations along the length of intramedullary stem 72. In some examples, fixation apertures 88A, 88B may be positioned at locations along the length of intramedullary stem portion 72 such that screws 83A and/or 83B are inserted through a cortical wall of a bone portion (e.g., proximal metatarsal bone portion 250) to engage fixation apertures 88A and/or 88B. For example, intramedullary stem portion 72 can be inserted into a bone portion and screws 83A and 83B then inserted through a cortical wall (e.g., medial cortical wall) of the bone portion to access and engage with fixation apertures 88A and 88B positioned in the medullary canal of the bone portion. The screws 83A and 83B may have a length that causes the ends of the screws to project into and/or through the opposite cortical wall of the bone portion (e.g., lateral cortical wall), when the screws are fully seated in fixation apertures 88A and/or 88B.

In some configurations, the proximal-most fixation aperture 88B is offset from the proximal most end of intramedullary stem portion 72 a distance 83 to define an unapertured region of intramedullary stem 72. This can provide an extended tail region of the plate offset from the proximal most fixation aperture 88B, which may help stabilize intramurally implant 70 within the intramedullary space of the bone portion into which intramedullary stem portion 72 is inserted.

In some configurations, intramedullary stem portion 72 includes at least one fixation aperture in a proximal-most portion of the stem, such as a proximal-most half of the stem, the proximal-most third of the stem, or the proximal-most quarter of the stem. This can provide a tail screw fixation aperture for receiving a tail screw in the tail portion of intramedullary stem 72.

FIG. 13F is a perspective of an example configuration of intramedullary implant 70 from FIGS. 13D and 13E where the intramedullary implant includes a fixation aperture 88C in the tail portion of intramedullary stem 72. In this example, intramedullary stem portion 72 is also illustrated as including fixation apertures 88A and 88B, each of which are offset distally from fixation aperture 88C. Intramedullary stem portion 72 may not include these additional fixation apertures or may include a different number or arrangement of fixation apertures extending through the stem. In either case, the tail screw fixation aperture 88C is configured to receive a screw 83C extending through the aperture.

Fixation apertures 88A-88C can be oriented relative to each other in a number of different ways, e.g., such that screws 83A-83C inserted through the respective fixation apertures are parallel to each other, converge toward each other, diverge away from each other, or include combinations thereof. In the illustrated arrangement, fixation apertures 88A and 88B are oriented relative to each other such that screws 83A and 83B extend parallel to each other and are angled proximally relative to the longitudinal axis of intramedullary stem 72. Further, in this example, fixation aperture 88C is orientated such that screw 83C extends substantially perpendicular to intramedullary stem portion 72 with the tips of screws 83A and 83B angled toward screw 83C. This can define a screw trajectory that orients screw 83C extending substantially perpendicularly relative to the longitudinal axis of proximal metatarsal portion 250, when inserted. It should be appreciated that discussion of screws herein is for purposes of discussion and other fixation members may be used without departing from the scope of the disclosure.

Configuring intramedullary implant 70 with the tail fixation aperture 88C and tail screw 83C may be beneficial for a variety of reasons. Such a feature may satisfy a clinician's perceived need for additional fixation, provide additional fixation for patients exhibiting for bone quality, and/or provide additional fixation for patients having a compromised cortical wall (e.g., lateral cortical wall). Additionally or alternatively, such a feature may provide an additional fixation location in the event that one or both of fixation apertures 88A, 88B are not used (e.g., because of degraded underlying bone, a tumor or infection, clinician preference).

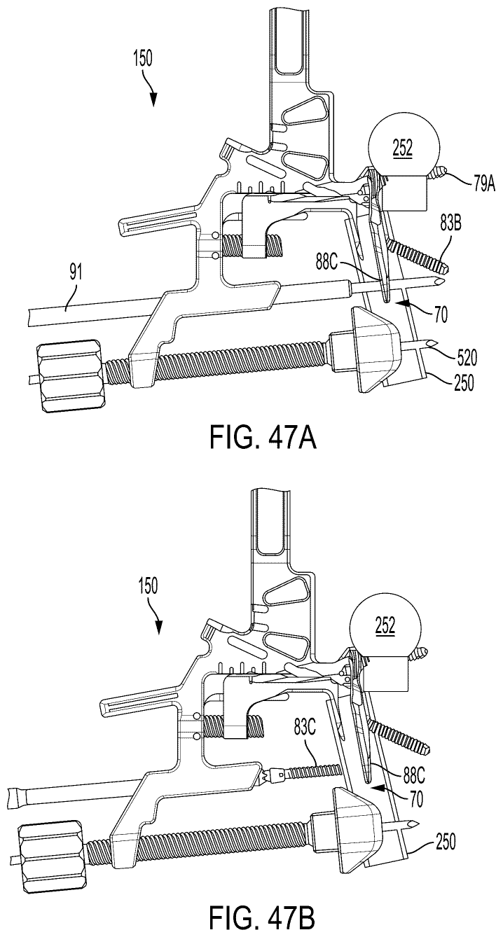

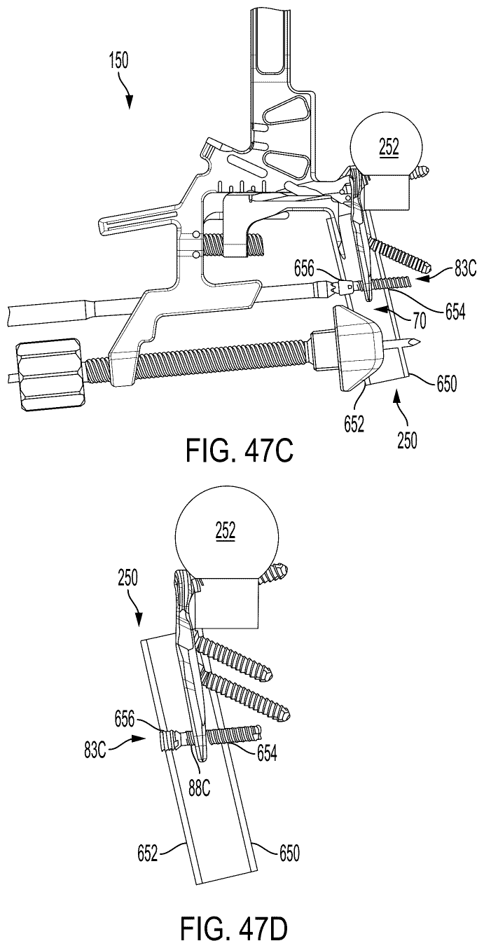

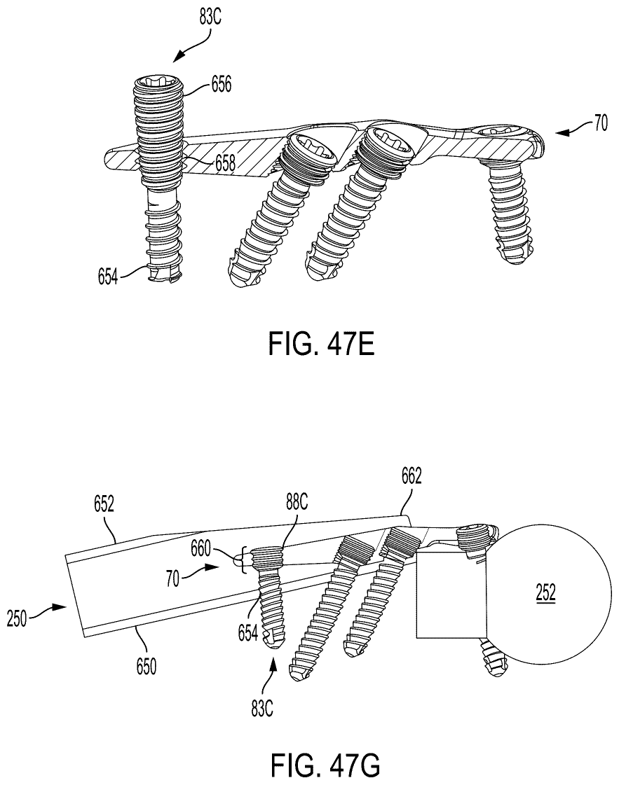

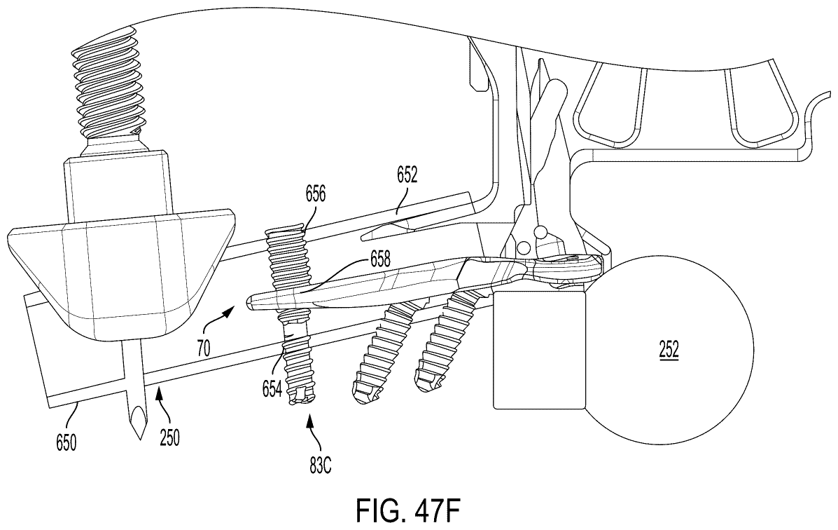

To install fixation screw 83C in fixation aperture 88C, a drill and/or tack be provided to form an opening in the bone that is aligned with fixation aperture 88C. FIG. 13G is a perspective view of intramedullary implant 70 from FIG. 13F illustrating an example tack 91 that can be used to form an opening for inserting a tail screw 88C. In this example, tack 91 is illustrated as having a wire tip (e.g., sized smaller than the diameter of screw 88C) and a thicker main body sized larger than fixation aperture 88C. Tack 91 may be used to form an opening in the bone for receiving fixation screw 83C and/or may be used for additional temporary fixation between intramedullary implant 70 and proximal metatarsal bone portion 250 before, during, and/or after realignment of distal metatarsal portion 252 (e.g., during frontal plane rotation of distal metatarsal portion 252). Additional details on example features and procedure steps for fixation screw 83C and fixation aperture 88C are discussed below with respect to FIGS. 47A-47G.

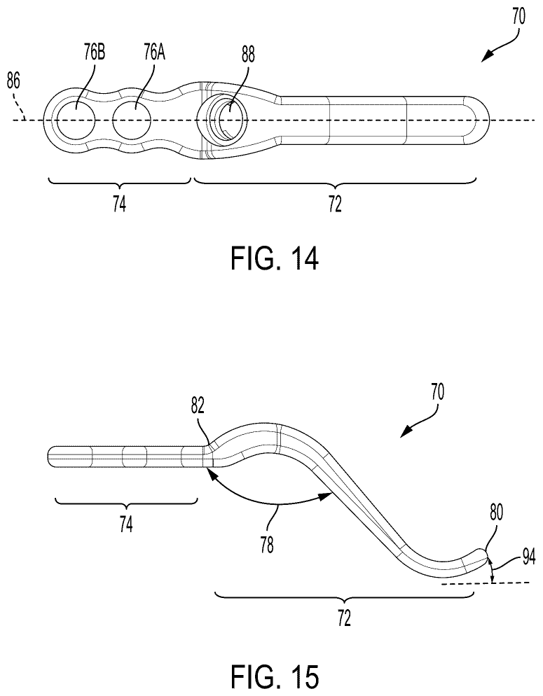

As noted, intramedullary implant 70 can have a variety of different configurations. FIGS. 14 and 15 are top and side views, respectively, of another example configuration of intramedullary implant 70. As shown in this example, plate 74 of intramedullary implant 70 is configured with two fixation apertures 76A, 76B positioned side-by-side lengthwise along the length of intramedullary implant 70, with each being substantially centered on an axis 86 bisecting the intramedullary implant lengthwise. In addition, in this example, intramedullary stem portion 72 defines a direction change the adjacent the first end 80 of the stem. In particular, the illustrated intramedullary stem is angled in a first direction relative to plate 74 at an angle 78 adjacent second end 82 it is angled in a second direction substantially opposite to the first direction at an angle 94 adjacent first end 80. In the illustrated orientation, intramedullary stem portion 72 is angled downwardly from plate 74 moving from second end 82 toward first end 80 until reaching a transition point at which the intramedullary stem changes direction and is angled upwardly relative to the adjacent region this step. This provides a generally “s-shaped” profile.

The clinician can insert intramedullary stem portion 72 of intramedullary implant 70 into a metatarsal bone portion a number of different ways. The clinician can manually grasp intramedullary implant 70 in advance the intramedullary stem portion 72 into the metatarsal bone portion (e.g., the pocket created in the metatarsal bone portion using the broach instrument 60). In some examples, an inserter is attached to intramedullary implant 70 and used to help install the intramedullary implant.

In use, a clinician can cut metatarsal 212 in a proximal metatarsal bone portion 250 and a distal metatarsal bone portion 252. The clinician can insert stem portion 72 of intramedullary implant 70 into a cut end face of proximal metatarsal bone portion 250, e.g., using an instrument attached to the intramedullary implant as discussed herein. The clinician can also position the one or more fixation apertures extending through plate portion 74 of intramedullary implant 72 overlying distal metatarsal bone portion 252, e.g., again while the intramedullary implant is attached to the instrument. The clinician can position the fixation apertures overlying distal metatarsal bone portion 252 with the plate portion 74 positioned over any desired surface of the distal metatarsal bone portion 252, such as a medial surface of the bone portion (e.g., medial half of the bone portion), a dorsal surface of the bone portion (e.g., dorsal half of the bone portion), and/or a dorsal-medial surface of the bone portion (e.g., dorsal-medial quadrant of the bone portion). The clinician can adjust the position of distal metatarsal bone portion 252 relative to proximal metatarsal bone portion 250 in one or more planes, as discussed herein, before, during, and/or after inserting stem portion 72 of intramedullary implant 70 into proximal metatarsal bone portion 250 and/or positioning the one or more fixation apertures extending through plate portion 74 of intramedullary implant 72 overlying distal metatarsal bone portion 252.

In some examples, stem portion 72 of intramedullary implant 70 has a length extending from a first end to a second end with plate portion 74 being positioned at the second end of the stem portion. Stem portion 72 can extend at an angle 78 relative to plate portion 42. The clinician can insert stem portion 72 of intramedullary implant 70 into proximal metatarsal portion 250 by at least positioning the first end of stem portion 72 in contact with a lateral cortical wall of the proximal metatarsal portion 250. The clinician may further position the second end of stem portion 72 in contact with a medial cortical wall of the proximal metatarsal portion 250. In some examples, the clinician positions the first end of stem portion 72 in contact with the lateral cortical wall of the proximal metatarsal portion 250 by at least positioning the first end of the stem portion projecting partially or fully through a thickness of the lateral cortical wall.

The clinician using intramedullary implant 70 can insert fixation members through each of the fixation aperture extending through plate portion 74 of the intramedullary implant and into distal metatarsal portion 252 to fixate a moved position of the second metatarsal portion relative to the first metatarsal portion. For example, the clinician may insert one or more screws extending through the fixation apertures extending through plate portion 74 and into the dorsal surface, medial surface, and/or dorsal-medial surface of distal metatarsal portion 252. The one or more screws may be locking screws non-locking screws, or combinations thereof. Additionally or alternatively, the clinician may insert one or more fixation members through one or more fixation aperture extending through stem portion 72 of the intramedullary implant and into proximal metatarsal portion 250 to further secure fixate the moved position of the second metatarsal portion relative to the first metatarsal portion. In some examples, the clinician may insert a screw through a fixation aperture extending through stem portion 72 of intramedullary implant 70 and through a cut end of proximal metatarsal bone portion 250. Additionally or alternatively, the clinician may insert a screw through a fixation aperture extending through stem portion 72 of intramedullary implant 70 and through one and/or two cortical walls of proximal metatarsal bone portion 250.

In some examples, one or more of the screws may have a length effective to position the tip of the screw in the medullary canal of distal metatarsal portion 252, when the screws are fully inserted into the corresponding fixation aperture of plate portion 74. These can be unicortical screws that only pass through a single cortex (e.g., medial cortex) of distal metatarsal portion 252. Additionally or alternatively, one or more of the screws may have a length effective to position the tip of the screw passing partially or fully through the opposite cortical wall of distal metatarsal portion 252, when the screws are fully inserted into the corresponding fixation aperture of plate portion 74. These can be bicortical screws that pass through two cortices (e.g., medial cortex and lateral cortex) of distal metatarsal portion 252. The length of such screws may be effective to position the tip of the screw in the intermetatarsal space between the lateral cortical wall of first metatarsal 212 and the medial cortical wall of second metatarsal 214.

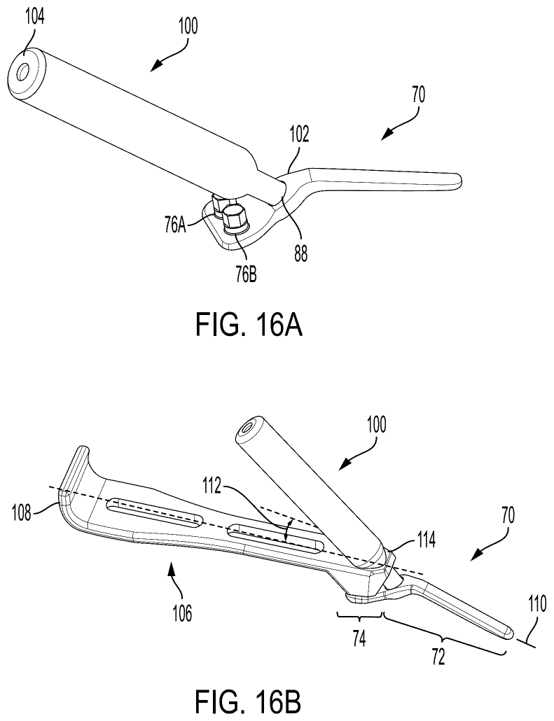

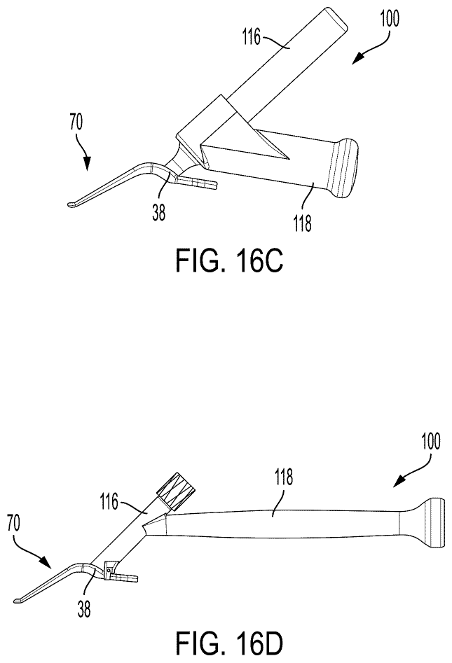

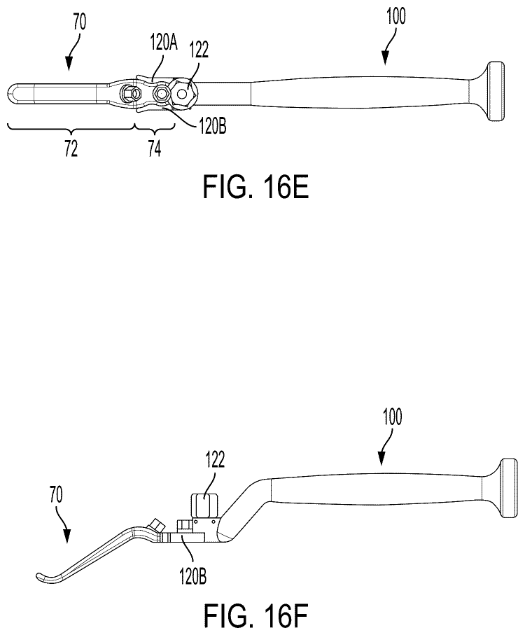

FIG. 16A is a perspective view of an example configuration of intramedullary implant 70 with an example inserter 100 attached to the implant. Inserter 100 can be defined by a body having a length extending from a first end 102 to a second end 104. Inserter can be removably attached to intramedullary implant 70 and used as an aid to manipulate positioning of the intramedullary implant and/or for applying an impaction force to help advance distal end 70 of the implant into the metatarsal bone portion. In the illustrated arrangement, inserter 100 is configured to engage one or more fixation apertures of intramedullary implant 70. For example, first end 102 of inserter 100 may be threaded and can be threadably engaged with screw receiving threading encircling the one or more fixation apertures of intramedullary implant 70. FIG. 16A illustrates inserter 100 threadingly attached to fixation aperture 88 extending through intramedullary stem 72.

In instances where the screw trajectory defined by fixation aperture 88 is not parallel to the longitudinal length of intramedullary stem 72, inserter 100 attached to fixation aperture 88 may be angled relative to the intramedullary stem. As a result, an axially directed force applied to inserter 100 may be off axis with an access bisecting intramedullary stem 72. Accordingly, in some implementations, the orientation of an inserter engaged with intramedullary implant 70 may be adjusted to better position the longitudinal axis of the inserter in line with a longitudinal axis bisecting intramedullary stem 72.

FIG. 16B is a perspective view of another example inserter configuration that can be used with intramedullary implant 70. FIG. 16B illustrates inserter 100 releasably engaged with intramedullary implant 70 as discussed with respect to FIG. 16A. In the example of FIG. 16B, however, a secondary inserter impaction arm 106 is engaged with intramedullary implant 70 to help with installation of the intramedullary implant into a metatarsal bone portion. As shown, secondary inserter impaction arm 106 extends outwardly from intramedullary implant 70 at an angle substantially the same as the angle at which intramedullary stem portion 72 extends. As a result in axes 108 extending along the longitudinal length of secondary inserter impaction arm 106 may be substantially co-linear with an axis 110 bisecting intramedullary stem 72. For example, an angle 112 formed between the two axes may be less than 45°, such as less than 30°, or less than 15°. This positioning can allow a clinician to impart an axially directed force on the end of secondary inserter impaction arm 106 to help impact in advance intramedullary stem portion 72 into a metatarsal bone portion.

When used, secondary inserter impaction arm 106 can be engaged with intramedullary implant 70 in a variety of different ways. In the illustrated example, secondary inserter impaction arm 106 defines an opening 114 that can be used to interlock the secondary inserter impaction arm to intramedullary implant 70. For example, secondary inserter impaction arm 16 can be positioned over plate 74 of intramedullary stem portion 72 with opening 114 positioned over fixation aperture 88. Inserter 100 can then be inserted through opening 114 of secondary inserter impaction arm 106 and engaged with fixation aperture 88, thereby sandwiching and interlocking secondary inserter impaction arm 106 between intramedullary implant 70 and inserter 100. Secondary inserter impaction arm 106 can be engaged with intramedullary implant 70 in a variety of different ways, such as being threadingly engaged with one or more of fixation apertures 76A, 76b; clamping to a side of intramedullary implant 70 across the thickness; and/or otherwise engaging with the implant. In some applications, secondary inserter impaction arm 106 can be used as the sole inserter without including inserter 100.

Inserter 100 coupled to intramedullary implant 70 can have a variety of different configurations. FIGS. 16C and 16D are images of an additional example configurations of inserter 100 in which the inserter is configured with different handle and/or impaction force receiving surfaces extending at different angles relative to intramedullary implant 70. In these examples, inserter 100 is shown configured with a first portion 116 extending generally axially away from the intramedullary stem of intramedullary implant 70. Inserter 100 is also illustrated in these examples as having a second portion 118 extending at an angle (e.g., an acute angle) relative to the first portion 116. This arrangement can provide the clinician with different services to hold an/or apply impaction forces to (e.g., via hand, a hammer, and/or other instrument) to help guide insertion of intramedullary implant 70 into a metatarsal bone portion.

FIGS. 16E and 16F are top and side views, respectively, of another example configuration of inserter 100 that can be releasably engaged with an example intramedullary implant 70. In this configuration, inserter 100 is configured to engage with sidewalls of intramedullary implant 70 to releasably hold the intramedullary implant for insertion (e.g., without engaging any fixation apertures of the implant). As illustrated, inserter 100 defines a pair of arms 120A, 120B with a receiving cavity formed between the arms that is configured to receive the end of intramedullary implant 70 (at least a portion of plate 74 of the implant). Inserter 100 can include an actuator 122 (e.g., threaded knob, slider) that controls the relative spacing between arms 120A, 120B. Intramedullary implant 70 can be inserted into the arms 120A, 120B of inserter 100 and actuator 122 engaged to compress the arms of the inserter against the sidewalls of the implant, thereby releasably retaining the implant to the inserter for use.

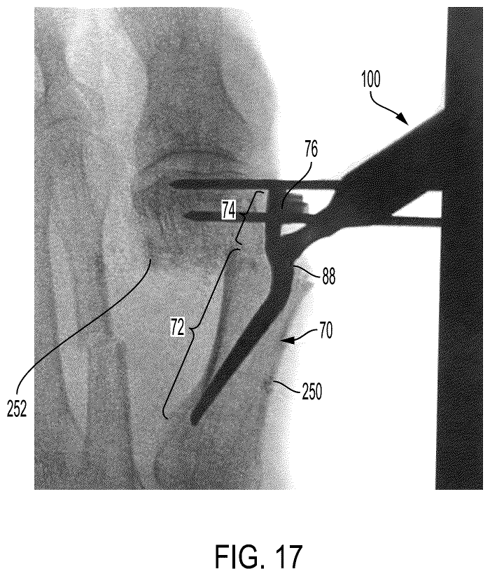

Independent of the configuration of inserter 100, and whether the clinician even uses an inserter to help insert intramedullary implant 70 into the metatarsal bone portion, the clinician can insert intramedullary stem portion 72 of the implant into a metatarsal bone portion. FIG. 17 is an example dorsal view fluoroscopic image showing an example configuration of implant 70 inserted into the metatarsal bone portion. In particular, intramedullary stem portion 72 of intramedullary implant 70 is inserted into proximal metatarsal bone portion 250. Intramedullary stem portion 72 can be inserted into proximal metatarsal bone portion 250 until the intramedullary stem is fully seated in the proximal bone portion (e.g., with fixation aperture 88 positioned on and/or immediately adjacent a cut end face 254 of proximal metatarsal bone portion 250). Depending on the configuration of intramedullary implant 70, intramedullary stem portion 72 may extend from the medial side of the medullary canal of proximal metatarsal bone portion 250 at the distal end of the implant (from which plate 74 extends) to a lateral side of the medullary canal of proximal metatarsal bone portion 250 at the proximal end of the implant. In some configurations, the proximal end of intramedullary stem portion 72 may project partially or fully through the thickness of the cortical wall of proximal metatarsal bone portion 250 (e.g., the lateral cortical wall of the bone). Plate 74 can be positioned adjacent to an/or in contact with the distal metatarsal bone portion 252 (e.g., such that fixation aperture(s) 76 are aligned with a medial sidewall of the distal metatarsal bone portion).

With further reference to FIG. 4, the example technique may involve installing one or more screws through one or more fixation apertures 88 of intramedullary implant 70 to help secure intramedullary stem portion 72 to proximal metatarsal bone portion 250 (step 18 in FIG. 4). While the example of FIG. 4 describes an example procedure order in which one or more screws are engaged with intramedullary stem portion 72 and proximal metatarsal bone portion 250 prior to inserting screws into plate 74 and distal metatarsal bone portion 252, the procedure order is not limited in this respect. In other applications, intramedullary implant 70 may be fixated to distal metatarsal bone portion 252 by inserting one or more screws through one or more fixation apertures 76 before and/or after inserting one or screws through the one or more fixation apertures 88 extending through intramedullary stem 72. Further, as discussed above, in other examples, an intramedullary implant 70 use during an osteotomy procedure may not utilize screws to supplementally secure intramedullary stem portion 72 to proximal metatarsal bone portion 250.

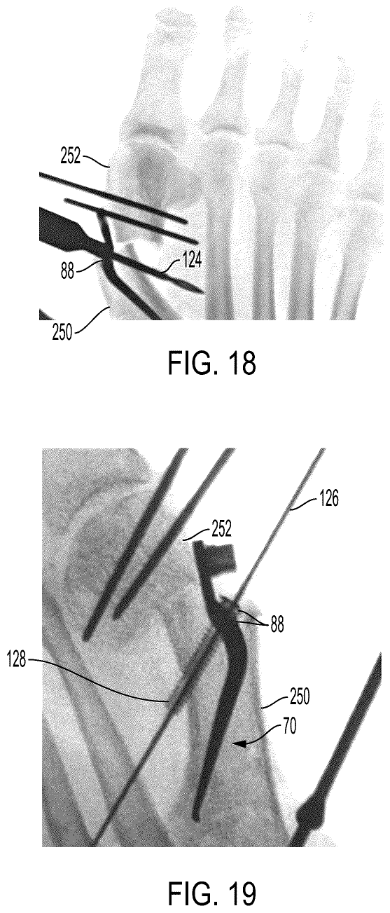

To secure intramedullary implant stem 72 to proximal metatarsal bone portion 250 using one or more screws, the clinician may predrill a hole through aperture 88 extending through intramedullary implant stem 72. In some examples, a drill guide is threadingly engaged with fixation aperture 88 to provide a drill guide channel extending outwardly from the intramedullary implant. In either case, a drill bit can be advanced through fixation apertures 88 along the trajectory defined by the fixation aperture into and/or through the underlying metatarsal bone portion (e.g., proximal metatarsal bone portion 250). FIG. 18 is an example dorsal view fluoroscopic image showing an example drill 124 inserted into fixation aperture 88 and through proximal metatarsal bone portion 250 underlying the fixation aperture. In some examples, a solid (non-cannulated) drill is used to form the hole in the proximal metatarsal bone portion 250 through fixation aperture 88.

After optionally predrilling a hole along the trajectory defined by fixation aperture 88, the clinician can install a fixation member (e.g., screw) through the fixation aperture and into the underlying metatarsal bone portion via of the predrilled hole. In some examples, the clinician inserts a screw directly through fixation apertures 88 without a supplemental guiding aid. In other examples, a clinician may insert a K-wire through fixation aperture 88 (optionally also through a drill guide threadingly inserted into the fixation aperture) and then guides a cannulated screw over the K-wire. FIG. 19 is an example dorsal view fluoroscopic image showing drill 124 (FIG. 18) removed from proximal metatarsal bone portion 250 and a K-wire 126 inserted through fixation aperture 88 of intramedullary implant 70. With K-wire 126 guiding the trajectory for inserting a fixation screw through fixation apertures 88, a clinician can guide a cannulated screw 128 over the K-wire 126 into fixation aperture 88. Utilizing a K-wire 126 and cannulated screw 128 can be useful to ensure that the clinician accurately targets fixation aperture 88 and seats the screw relative to intramedullary implant 70 during installation. That being said, in other examples, a non-cannulated screw may be used in lieu of a cannulated screw to secure intramedullary stem portion 72 to proximal metatarsal bone portion 250.

In different implementations, screw 128 to be inserted into fixation aperture 88 of intramedullary stem portion 72 can be a locking screw or a nonlocking screw (e.g., compression screw). A locking screw can have threading extending about the head of the screw that interlocks with complementary threading extending about fixation aperture 88. Screw 128 can have a variety of lengths. In some examples, screw 128 is sized such that the end of the screw opposite the head that contacts intramedullary implant 70 resides within the medullary canal and/or cortical wall of proximal metatarsal bone portion 250, when the screw is fully inserted into the intramedullary implant. In some examples, screw 128 is sized such that the end of the screw opposite the head that contacts the plate of intramedullary implant 70 projects through the cortical wall surface (e.g., lateral cortical wall surface) of proximal metatarsal bone portion 250, when the screw is fully inserted into the intramedullary implant. When so configured, the end of the screw may reside within the intermetatarsal space between proximal metatarsal bone portion 250 and the adjacent second metatarsal, as illustrated in the example of FIG. 19.

The example technique of FIG. 4 also involves moving distal metatarsal bone portion 252 relative to proximal metatarsal bone portion 250, e.g., to correct a deformity such as a bunion deformity (step 20 in FIG. 4). For example, the clinician can shift distal metatarsal bone portion 252 in the transverse plane (e.g., to move the distal metatarsal bone portion laterally), rotate the distal metatarsal bone portion in the frontal plane, and/or shift the distal metatarsal bone portion in the sagittal plane. While the example of FIG. 4 describes an example procedure order in which distal metatarsal bone portion 252 is realigned following insertion of the intramedullary stem into proximal metatarsal bone portion 250, it should be appreciated the procedure is not limited to this order. In practice, distal metatarsal bone portion 252 can be moved in one or more planes prior to attaching intramedullary implant 70 to either or both metatarsal bone portions, while attaching the intramedullary implant to either or both metatarsal bone portions, and/or after attaching the intramedullary implant to either or both metatarsal bone portions.

For example, realignment of distal metatarsal bone portion 252 may occur through various stages of the surgical procedure. The distal metatarsal bone portion 252 may be moved in the transverse plane (e.g., laterally) relative to proximal metatarsal bone portion 250 prior to and/or while inserting intramedullary implant 70 into proximal metatarsal bone portion 250. For example, after cutting first metatarsal 210 into the proximal metatarsal bone portion 250 and distal metatarsal bone portion 252, the clinician can shift distal metatarsal bone portion 252 laterally to at least partially expose the cut end face of proximal metatarsal bone portion 250. The clinician can grasp a K-wire inserted into distal metatarsal bone portion 252 (e.g., a K-wire used to secure the bone preparation guide to the distal metatarsal bone portion, with the K-wire remaining in the bone portion after removing the bone preparation guide) to move the distal bone portion laterally. Additionally or alternatively, the clinician can move distal bone portion 252 laterally when inserting broach instrument 60 or another instrument and/or intramedullary stem portion 72 of intramedullary implant 70 into the cut end face of proximal metatarsal bone portion 250.

The distance distal metatarsal bone portion 252 is moved laterally in the transverse plane relative to proximal metatarsal bone portion 250 may be set based on the offset distance between the end of intramedullary stem portion 72 of intramedullary implant 70 and the plate 74 of the intramedullary implant. In some examples, distal metatarsal bone portion 252 is shifted laterally in the transverse plane so less than half of the cut end face of the distal metatarsal bone portion is positioned over and/or in contact with the cut end face of the proximal metatarsal bone portion. For example, distal metatarsal bone portion 252 may be shifted laterally so the medial-most half or less of the distal metatarsal bone portion is positioned over and/or in contact with a lateral portion of the cut end face of proximal metatarsal bone portion 250, such as the medial-most third or less of the distal metatarsal bone portion, the medial-most quarter or less of the distal metatarsal bone portion, or the medial-most fifth or less of the distal metatarsal bone portion.

Distal metatarsal bone portion 252 can be moved in one or more other planes relative to proximal metatarsal bone portion 250 in addition to or in lieu of moving the bone portion in the transverse plane. For example, before, after, and/or while engaging intramedullary stem portion 72 with proximal metatarsal bone portion 250 as discussed in connection with steps 16 and 18 of FIG. 4, distal metatarsal bone portion 252 may be rotated in the frontal plane and/or plantar flexed or dorsiflexed in the sagittal plane.

The clinician may move distal metatarsal bone portion 252 in one or more planes with and/or without the aid of a bone positioning device. In some examples, the clinician grasps one or more K-wires inserted into distal metatarsal bone portion 252 (e.g., extending percutaneously out of the skin of the patient) and manipulates the position of the distal metatarsal bone portion in one or planes. Additionally or alternatively, the clinician may engage a bone positioning device with distal metatarsal bone portion 252 that can be controlled (e.g., actuated) to move the distal metatarsal bone portion in one or planes.

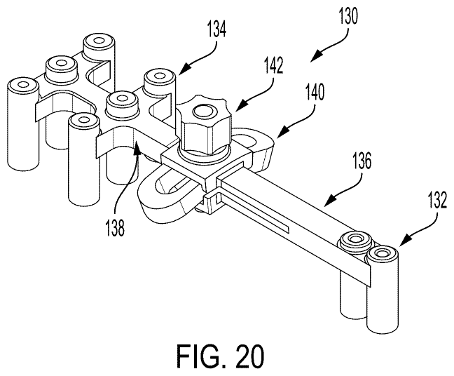

FIG. 20 is a perspective view of an example bone positioning device 130 that can be used to control positioning of distal metatarsal bone portion 252 relative to proximal metatarsal bone portion 250. Bone positioning device 130 in the illustrated example has a body defining one or more pin receiving apertures 132 on a first side of the body in the one or more additional pin receiving apertures 134 on a second side of body. Each side of bone positioning device 130 can include any desired number and/or arrangement of pin receiving apertures. For example, one or both sides of bone positioning device 130 may include two or more pin receiving apertures positioned side-by-side and separated from each other lengthwise along the body of the bone positioning device and/or two or more pin receiving apertures positioned side-by-side and separated from each other widthwise along the body of the bone positioning device.

Bone positioning device 130 can be engaged with distal metatarsal bone portion 252 by placing the one or more pin receiving apertures 132 over one or more corresponding K-wires inserted into the distal metatarsal bone portion (and/or positioning the pin receiving apertures over the distal metatarsal bone portion and then inserting K wires there through). When using a bone preparation guide, one or more K-wires used to secure the bone preparation guide to distal metatarsal bone portion 252 can be left in the bone portion after lifting the bone preparation guide off the K-wires. These one or more K-wires can then be used to manipulate the position of the distal metatarsal bone portion by hand and/or through engagement of the bone positioning device 130 with the K-wires. Bone positioning device 130 can be engaged with proximal metatarsal bone portion 250 by placing the one or more pin receiving apertures 134 over one or more corresponding K-wires inserted into the proximal metatarsal bone portion (and/or positioning the pin receiving apertures over the proximal metatarsal bone portion and then inserting K wires there through).

Bone positioning device 130 can include a distal body portion 136 and a proximal body portion 138 that are configured to move relative to each other via a connection 140, such as a sliding connection. With the bone positioning device 130 pinned to proximal metatarsal bone portion 250 and distal metatarsal bone portion 252, the clinician can move distal body portion 136 relative to proximal body portion 138, e.g., to adjust the rotational position of the body portions relative to each other and correspondingly the rotational position of the distal metatarsal bone portion 252 relative to the proximal metatarsal bone portion 250. This can adjust the rotational position of distal metatarsal bone portion 252 in the frontal plane. When the distal metatarsal bone portion 252 is moved to a desired position using bone positioning device 130, the clinician may engage a lock 142 to lock the distal to proximal portions of the bone positioning device relative to each other and, correspondingly, the metatarsal bone portions attached thereto. Additionally or alternatively, the clinician may insert one or more wires through distal metatarsal bone portion 252 and into an adjacent bone (e.g., adjacent second metatarsal) to temporarily secure and hold the moved position of the distal metatarsal bone portion.

The example technique of FIG. 4 may also involve installing one or more screws through one or more fixation apertures 76 of intramedullary implant 70 to secure intramedullary plate portion 74 to distal metatarsal bone portion 252 (step 22 in FIG. 4). Again, while the example of FIG. 4 describes an example procedure order in which one or more screws are engaged with intramedullary implant plate 74 and distal metatarsal bone portion 252 after inserting one or more screws into intramedullary stem portion 72 and proximal metatarsal bone portion 250, the procedure order is not limited in this respect. In other applications, intramedullary implant 70 may be fixated to distal metatarsal bone portion 252 before inserting one or more screws through one or more fixation apertures 88 and into proximal metatarsal bone portion 250.

To secure intramedullary implant plate 74 to distal metatarsal bone portion 252 using one or more screws, the clinician may predrill a hole through apertures 76 extending through intramedullary implant plate 74. In some examples, drill guides are threadingly engaged with fixation apertures 76 to provide a drill guide channel extending outwardly from the intramedullary implant. In some examples, a drill guide associated with an instrument holding intramedullary implant 70 is used to guide a drill bit. In either case, a drill bit can be advanced through fixation apertures 76 along the trajectory defined by each fixation aperture into the underlying distal bone portion 252.

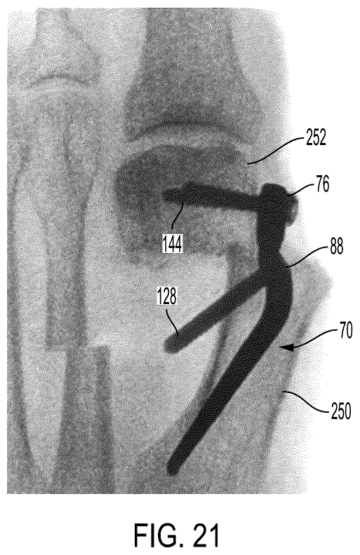

After optionally predrilling holes along the trajectory defined by fixation apertures 76, the clinician can install fixation members (e.g., screws) through the fixation apertures and into the underlying metatarsal bone portion. Each screw inserted through a corresponding fixation aperture 76 may be a locking and/or a nonlocking screw (e.g., compression screw). A locking screw can have threading extending about the head of the screw that interlocks with complementary threading extending about fixation aperture 76. Each screw inserted through a corresponding fixation aperture 76 can have a variety of lengths. In some examples, the screw is sized such that the end of the screw opposite the head that contacts the plate 74 of intramedullary implant 70 resides within the medullary canal of distal metatarsal bone portion 252 and does not pierce through the opposed (lateral) cortical wall of the distal metatarsal bone portion, when the screw is fully seated relative to intramedullary implant 70. FIG. 21 is an example dorsal view fluoroscopic image showing screws 144 inserted through fixation apertures 76 of intramedullary implant 70 to attach the implant to distal metatarsal bone portion 252. In other examples, the screw is sized such that the end of the screw opposite the head that contacts the plate 74 of intramedullary implant 70 extends through the cortical wall (e.g., lateral cortical wall) of distal metatarsal bone portion 252, when the screw is fully seated relative to intramedullary implant 70.

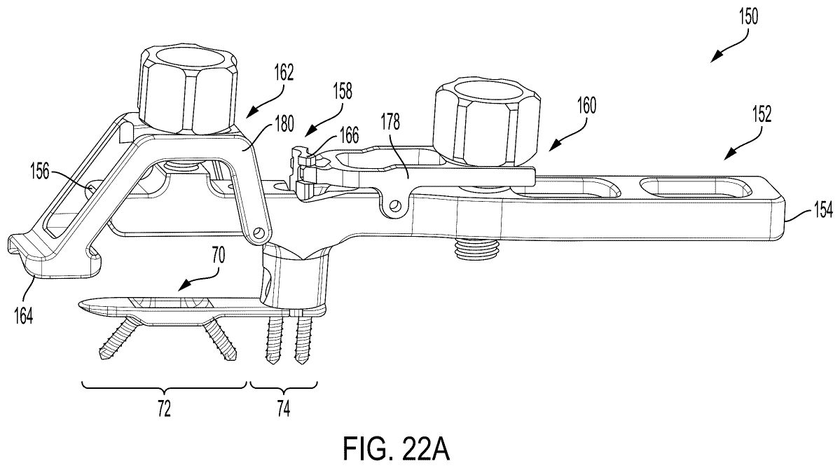

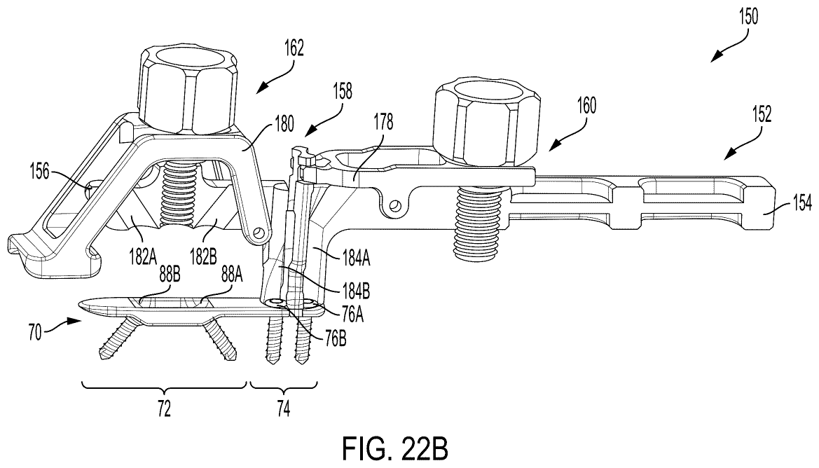

As discussed above, a variety of different inserter instruments and/or bone positioner devices can be used during an osteotomy procedure according to the disclosure. FIGS. 22A and 22B (collectively referred to as “FIG. 22”) are a perspective view and a sectional view of an example instrument 150 that can be used to guide installation of an intramedullary implant 70 and/or positioning of the distal metatarsal bone portion 252 relative to the proximal metatarsal bone portion. Instrument 150 may be referred to as an intramedullary implant insertion and positioning instrument.

As shown in the illustrated example of FIG. 22, instrument 150 can have a body 152 extending from a first end 154 to a second end 156. Body 152 can be operatively connected to an implant attachment member 158 that is releasably engageable with intramedullary implant 70. In use, implant attachment member 158 can be engaged with intramedullary implant 70 to stably hold the implant, allowing the clinician to manipulate instrument 150 and thereby guide positioning of the implant relative to proximal metatarsal bone portion 250 and/or distal metatarsal bone portion 252. Instrument 150 can include a mechanism 160 operatively connected to implant attachment member 158 and operable to engage and/or disengage the implant attachment from intramedullary implant 70.

In some examples, such as the illustrated example of FIG. 22, instrument 150 includes a bone positioning device 162 that can apply a force between one or more bone portions contacted by the bone positioning device and the instrument. For example, bone positioning device 162 can define a contact surface 164 that can contact a bone portion (e.g., directly or indirectly via an overlying skin layer that the contact surface touches). For example, in the illustrated arrangement, bone positioning device 162 is illustrated as being positioned at the second end 156 of body 152 of instrument 150. Implant attachment member 158 is positioned offset along the longitudinal axis of body 152 toward first end 154. As a result, contact surface 164 of bone positioning device 162 can be positioned over and/or distally beyond the intramedullary stem portion 72 of the implant engaged by implant attachment member 158. In use, bone positioning device 162 can be actuated to apply a force between a bone portion underlying contact surface 164 of body 152 of the instrument.

The force applied by bone positioning device 162 can be effective to help realign distal metatarsal bone portion 252 relative to proximal metatarsal bone portion 250. Additionally or alternatively, the force applied by bone positioning device 162 can help the instrument stably engage foot 200 during a surgical procedure. For example, the proximally-located bone positioning device 162 can provide a counterforce to the end of the instrument carrying an implant inserted into proximal metatarsal bone portion 250. The pressure between contact surface 164 and the underlying bone portion (e.g., skin covering the bone portion) can help stabilize instrument 150 during the surgical procedure. The clinician can visualize the bone portions under imaging (e.g., fluoroscopy) with instrument 150 attached. The clinician can evaluate the relative position of distal metatarsal bone portion 252 to metatarsal bone portion 250 and/or the position of intramedullary implant 70 relate to one or both bone portions under imaging. The clinician can adjust the position of distal metatarsal bone portion 252 relative to metatarsal bone portion 250 (e.g., by hand and/or through the use of instrument 150) and/or the position of the implant relative to one or both bone portions until desired positioning is achieved.

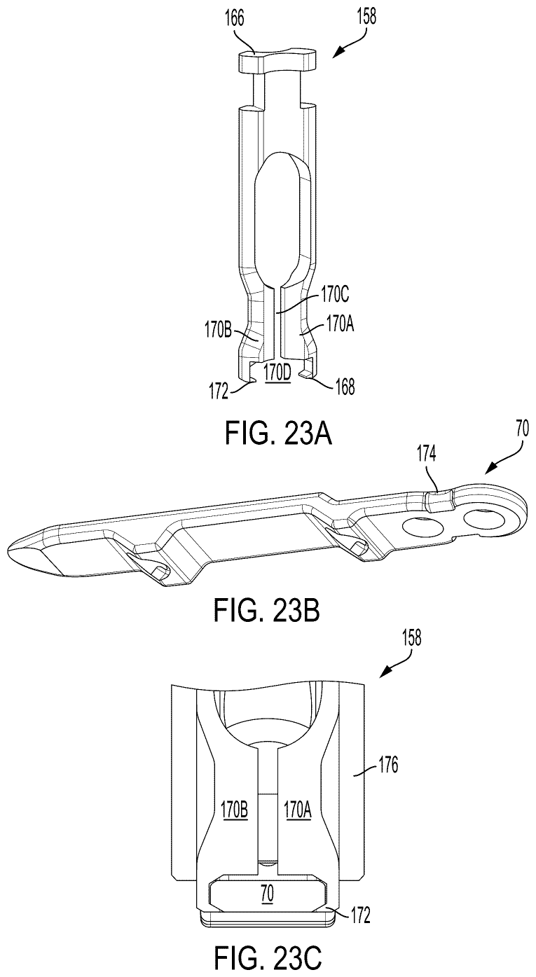

Implant attachment member 158 can have a variety of different configurations that function to releasably engage intramedullary implant 70. FIG. 23A is a perspective view of an example configuration of implant attachment member 158 that can be used on instrument 150. In this example, implant attachment member 158 defines a body extending from a first end 166 to a second end 168. The second end 168 of the body is divided into a pair of arms 170A and 170B separated by a space 170C between the two arms. The terminal end of each arm can define a receiving cavity 170D configured (e.g., sized and/or shaped) to receive intramedullary implant 70 therein. In some examples, the terminal end of each arm 170A, 170B defines an inward projecting foot 172 extending at least partially under the receiving cavity 170D into which intramedullary implant 70 can be received. Arms 170A, 170B can be separated from each other a distance sufficient to insert intramedullary implant 70 into and remove the intramedullary implant from the receiving cavity 170D. Arms 170A, 170B can also be drawn together a distance effective to grasp and retain intramedullary implant 70 inserted into receiving cavity 170D therein.

In some examples, intramedullary implant 70 is inserted into receiving cavity 170D and arms 170A, 170B then pulled and/or pushed together to cause the arms to press against opposed sidewalls of the intramedullary implant. Intramedullary implant 70 may or may not be configured with receiving channels on one or both opposed sidewalls of the intramedullary implant about which arms 170A, 170B are configured to grasp. For example, FIG. 23B is a perspective view of an example configuration of an intramedullary implant 70 where the opposed sidewalls of the implant are configured with channels 174 to define a recessed space relative to a remainder of the sidewall. Each channel 174 may be configured to receive a corresponding one of arms 170A, 170B from implant attachment member 158.

FIG. 23C is a side sectional view of implant engagement member 158 showing arms 170A, 170B grasping opposed sidewalls of intramedullary implant 70 with feet 172 extending at least partially under the implant. To engage and disengage implant engagement member 158 from intramedullary implant 70, the implant engagement member may be actuated relative to stationary and/or movable opposed wall surfaces 176. The distance between opposed wall surfaces 176 may be set and/or adjusted (upward and downwardly or inwardly and outwardly) to cause arms 170A, 170B to move toward each other a distance effective to grasp and retain intramedullary implant 70.

With further reference to FIG. 22, instrument 150 may include mechanism 160 engaging and disengaging implant attachment member 158. In the illustrated arrangement, mechanism 160 is shown as a knob connected via a threaded shaft to body 152 of instrument 150. The knob and/or threaded shaft are coupled to first end 166 of implant attachment member 158 via a pivoting body 178. Engaging the knob of mechanism 160 in one direction can cause pivoting body 178 to push implant attachment member 158 away from body 152, thereby moving arms 170A, 170 outwardly relative to wall surfaces 176 (FIG. 23C) and allowing the arms to spread apart to insert or remove implant 70 from between the arms. Engaging the knob of mechanism 160 in the opposite direction can cause pivoting body 178 to pull implant attachment member 158 toward body 152, thereby moving arms 170A, 170 inwardly relative to wall surfaces 176 (FIG. 23C) and causing the arms to push together based on the spacing between wall surfaces 176 to retain implant 70 between the arms.

In use, a clinician can attach intramedullary implant 70 to implant attachment member 158 of instrument 150 and use the instrument to guide installation of the implant into one or more metatarsal bone portions. For example, as discussed above in connection with inserter 100, the clinician can grasp the instrument and use it to guide intramedullary stem portion 72 into proximal metatarsal bone portion 250. When configured with bone positioning device 162, the bone positioning device can be to apply a force helping to position intramedullary implant 70 and/or to control the positioning of distal metatarsal bone portion 252 relative to the proximal metatarsal bone portion.

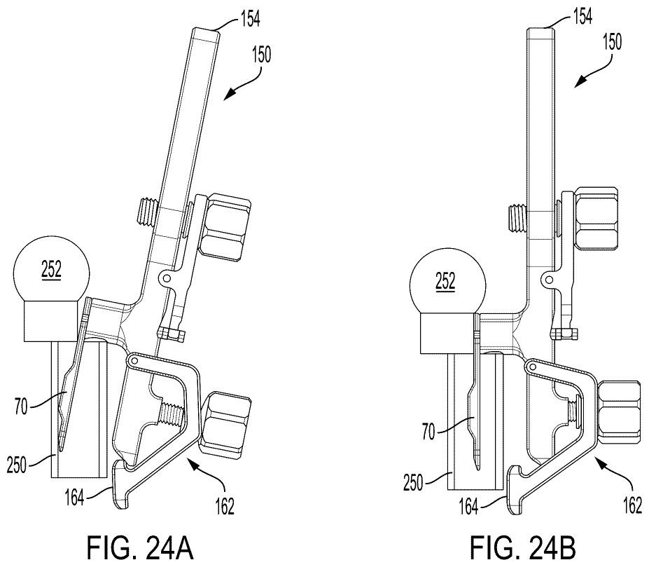

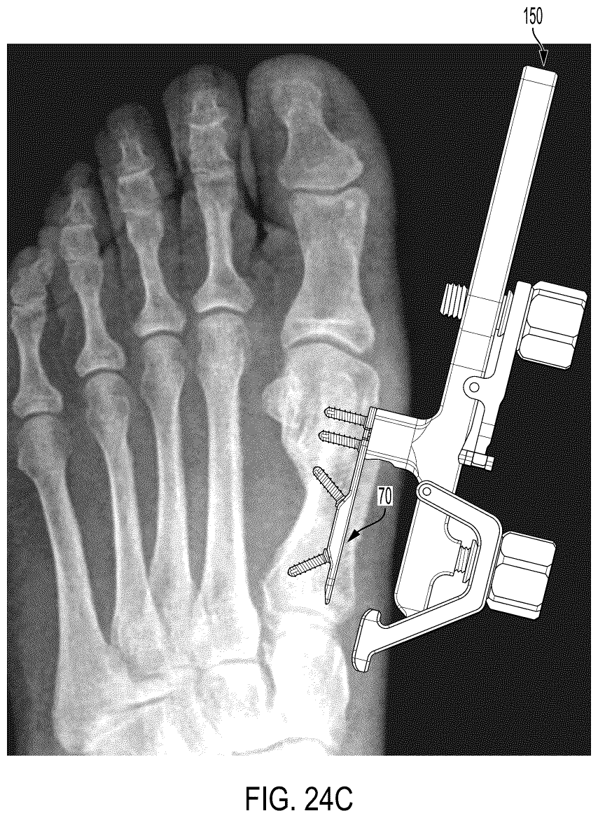

FIGS. 24A and 24B illustrate example procedural steps in which bone positioning device 162 can help facilitate installation of intramedullary implant 70 and/or positioning of distal metatarsal bone portion 252 relative to proximal metatarsal bone portion 250. As shown in FIG. 24A, instrument 150 can be used to guide intramedullary implant 70 into proximal metatarsal bone portion 250, which may result in the stem of the implant being inserted at an angle relative to the cut end face of the proximal metatarsal bone portion. Bone positioning device 162 can be actuated as shown in FIG. 24B to apply a force that results in realignment of intramedullary implant 70 within proximal metatarsal bone portion 250 and/or shifting of distal metatarsal bone portion 252 laterally. Contact surface 164 of bone positioning device 162 can apply a force to proximal metatarsal bone portion 250, medial cuneiform 222, and/or another bone portion. Bone positioning device 162 can apply force through contact surface 164 to the medial side of the bone portion, e.g., resulting in the end 154 of instrument 150 opposite contact surface 164 rotating laterally. This can change the angular orientation of intramedullary implant 70 within the medullary canal of proximal metatarsal bone portion 250. Additionally or alternatively, this can move distal metatarsal bone portion 252 laterally through a laterally-directed force between the plate portion of the intramedullary implant 70 and the distal metatarsal bone portion 252 (as the end 154 of instrument 150 opposite contact surface 164 rotates laterally). Additionally or alternatively, application of the force can pull proximal metatarsal bone portion 250 medially relative to distal bone portion 242 to increase the offset between the cut end faces of the two bone portions. FIG. 24C is an example image of a foot showing intramedullary implant 70 fixated to proximal and distal metatarsal bone portions and instrument 150 still secured to the bone portions.

Bone positioning device 162 can have a variety of different configurations. In the illustrated arrangement, device 162 is shown as including a knob connected via a threaded shaft to body 152 of instrument 150. The knob and/or threaded shaft are coupled to a pivoting body 180 defining contact surface 164. Engaging the knob of device 162 in one direction can cause pivoting body 180 to push contact surface 164 away from body 152, e.g., thereby causing the first end 154 of body 152 to rotate laterally. Engaging the knob of device 162 in the opposite direction can cause pivoting body 180 to pull contact surface 164 toward body 152.

With further reference to FIG. 22, instrument 150 can include a variety of features in addition to or in lieu of bone positioning device 162. In some examples, instrument 150 defines one or more screw insertion apertures that are coaxially aligned one or more of a corresponding fixation apertures of intramedullary implant 70 (when the intramedullary implant is attached to implant attachment member 158). For example, with reference to FIG. 22B, instrument 150 may define one or more screw insertion apertures 182 (e.g., apertures 182A, 182B) aligned with the trajectory of the one or more fixation apertures 88 extending through intramedullary implant stem 72. Additionally or alternatively, instrument 150 may define one or more screw insertion apertures 184 (e.g., 184A, 184B) aligned with the trajectory of the one or more fixation apertures 76 extending through intramedullary implant plate 74.

Each screw insertion aperture 182, 184 may be an opening extending through a portion of instrument 150 (e.g., extending through body 152) having a length and a direction. An axis extending parallel to the length of the screw insertion aperture may be co-axially aligned with a corresponding fixture aperture defined by intramedullary implant 70. This can help the clinician to guide insertion of fixation screws when installing intramedullary implant 70. For example, after suitably positioning intramedullary implant 70 relative to proximal metatarsal bone portion 250 and/or distal metatarsal bone portion 252, the clinician can drill pilot holes and/or guide fixation screws through each screw insertion aperture of instrument 150 (while intramedullary implant 70 remains engaged with the instrument). This can reduce or eliminate the challenge the clinician may otherwise face accurately locating each fixation aperture of intramedullary implant 70 after insertion of the intramedullary implant into proximal metatarsal bone portion 250.

In general, intramedullary implant 70 can have any desired number and orientation of fixation apertures configured to receive screws to secure the intramedullary implant to proximal metatarsal bone portion 250 and/or distal metatarsal bone portion 252. In some examples, intramedullary implant 70 includes one or more fixation apertures 88 positioned along intramedullary stem portion 72 that define angled screw insertion trajectories relative to one or more fixation apertures 76 positioned along intramedullary implant plate 74 and the screw insertion trajectories defined thereby. Angled screw insertion trajectories can increase the length of engagement between the bone portion and screw, e.g., as compared to a perpendicularly inserted screw.

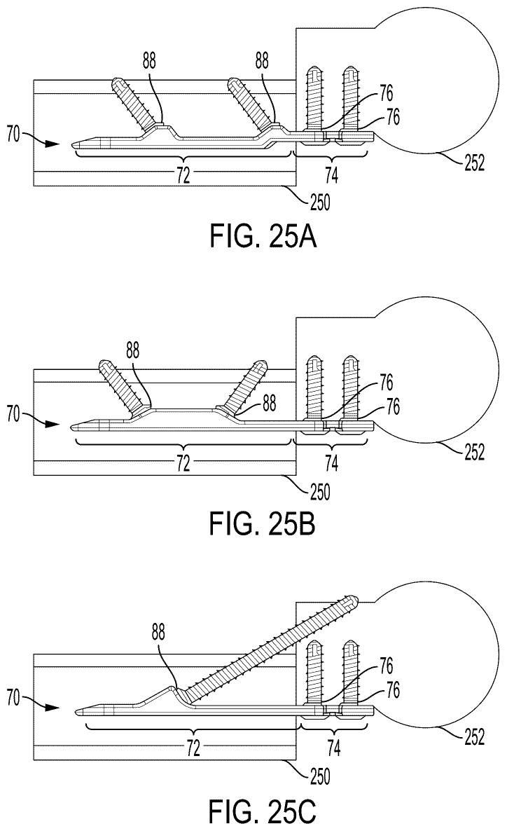

FIGS. 25A-25C illustrate example screw trajectory configurations that can be used for intramedullary implant 70. In each of the examples, the fixation apertures 76 positioned on intramedullary implant plate 74 are illustrated as defining screw insertion trajectories perpendicular to the underlying distal metatarsal bone portion 252. However, a different number or arrangement (e.g., screw trajectory) can be used.

FIG. 25A illustrates intramedullary implant 70 including at least one fixation aperture 88 on intramedullary stem portion 72 defining a screw insertion trajectory that orients the screw to diverge away from distal metatarsal bone portion 252 (such that the end of the screw is positioned more proximally of distal metatarsal bone portion 252 than the head of the screw engaging the implant). FIG. 25B illustrates intramedullary implant 70 including one fixation aperture 88 on intramedullary stem portion 72 defining a screw insertion trajectory that orients a screw to diverge away from distal metatarsal bone portion 252 and one fixation aperture 88 on intramedullary stem portion 72 defining a screw insertion trajectory that orients a screw to converge toward distal metatarsal bone portion 252. When so configured, one screw may be inserted so the end of the screw is positioned more proximally of distal metatarsal bone portion 252 than the head of the screw engaging the implant, and the other screw may be inserted so the end of the screw is positioned more distally to distal metatarsal bone portion 252 than the head of the screw engaging the implant.

FIG. 25C illustrates intramedullary implant 70 including one fixation aperture 88 on intramedullary stem portion 72 defining a screw insertion trajectory that orients a screw to converge toward distal metatarsal bone portion 252. In this example, the screw inserted through fixture aperture 88 is positioned at an orientation and has a length effective to project through the lateral cortical wall of proximal metatarsal bone portion 250 and into and/or through distal metatarsal bone portion 252.

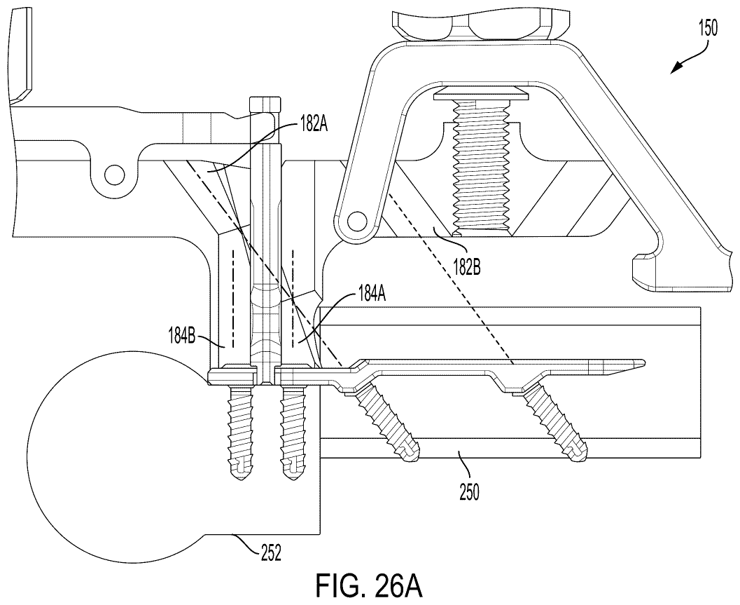

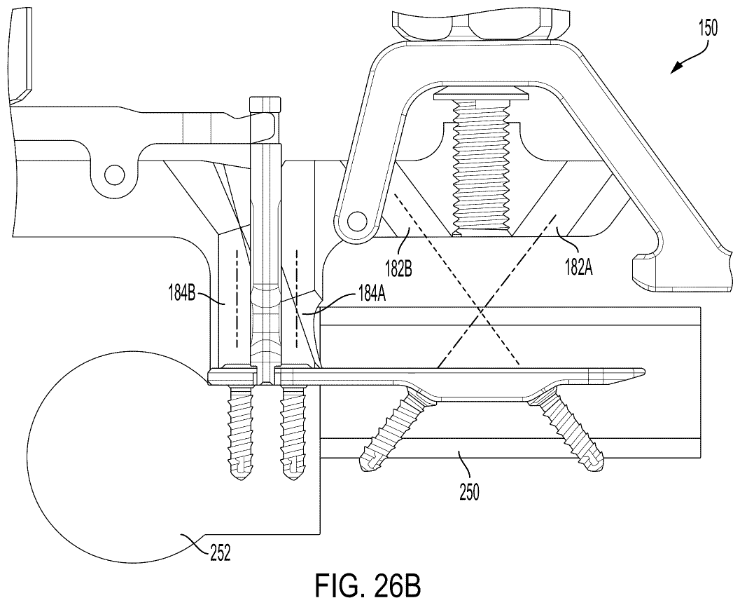

Independent of the configuration of intramedullary implant 70 and the number and arrangement of fixation apertures defined by the intramedullary implant, instrument 150 can define screw insertion apertures 182, 184 corresponding to each of the fixation apertures of the intramedullary implant. For example, FIG. 26A illustrates a configuration of instrument 150 defining screw insertion apertures aligned with the fixation aperture configuration of the intramedullary implant of FIG. 25A. FIG. 26B illustrates a configuration of instrument 150 defining screw insertion apertures aligned with the fixation aperture configuration of the intramedullary implant of FIG. 25B.

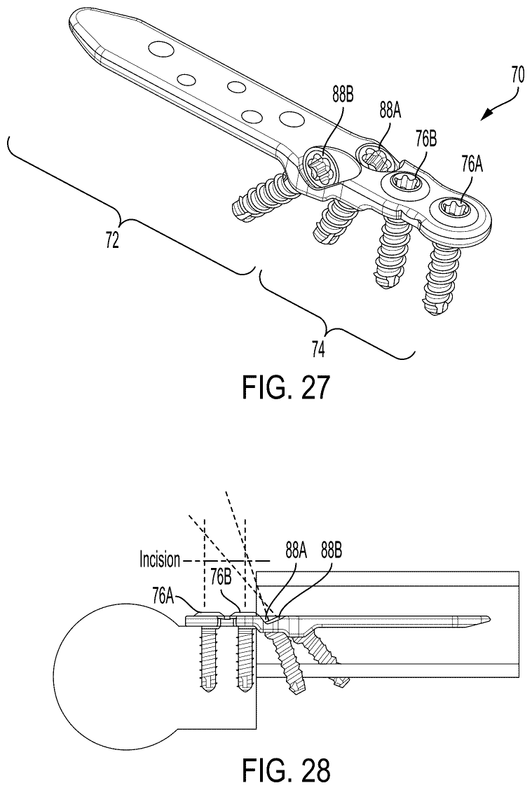

FIG. 27 is an illustration of another example configuration of intramedullary implant 70 showing example fixation aperture and screw trajectory orientations. In this example, intramedullary implant stem 72 includes fixation apertures 88 positioned on the distal end of the stem adjacent intramedullary implant plate 74. The orientation of fixation apertures 88 may be configured to cause the screws inserted therethrough to angularly diverge from each other. For example, the two screws inserted through fixation apertures 88 may be inserted at different relative angles to each other. In some examples, the screws may diverge laterally and/or proximally and/or may converge (e.g., cross) when viewed axially. Positioning fixation apertures 88 on the distal end of intramedullary implant stem 72 may allow all the fixation apertures of intramedullary implant 70 to be accessed through a single incision location, as schematically illustrated in FIG. 28.

Each fixture aperture defined by intramedullary implant 70 may be configured to receive a locking screw and/or non-locking screw. When configured to receive a locking screw, the fixation aperture can include threading extending about the perimeter of the fixation aperture into which the head of the locking screw can threadingly engage. With a typical locking screw, the threads on the head of the screw may have a constant diameter (pitch), meaning the inner/male thread can be advanced anywhere along the outer/female thread (because the threads are uniform). In some configurations, a flange or cap may be added to the screw such that, as the inner thread is advanced, the screw will stop advancing as soon as the head bottoms out on the external surface of the outer thread/plate.

In some implementations according to the present disclosure, one or more flange-less/cap-less locking screws may be used to fixate intramedullary implant 70 to an underlying bone portion. The screw may have a head defining threading configured to engage with complementary threading extending about the fixation aperture of intramedullary implant 70. The last partial thread on the screw may get progressively shallower. As the screw is threaded into the intramedullary implant fixation aperture, once the tapered portion interacts with the female threads, the male thread can jam and prevent further advancement of the screw. The taper may be adjusted such that when fully jammed, the top surface of the head may be flush with the body of the intramedullary implant, thereby reducing the profile as compared to when using a screw with cap.

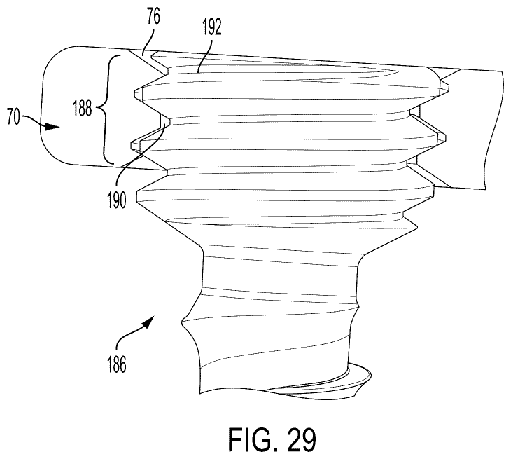

FIG. 29 is a side sectional illustration of an example cap-less locking screw configuration with a shallower terminal thread. As shown in this example, screw 186 is inserted into intramedullary implant 70. Screw 186 has a locking head 188 that defines a region of thread width 190 that threadably engages with corresponding threading extending about the fixation aperture of the implant. Screw 186 also has a terminal thread region 192 of reduced width, e.g., that gets progressively shallower. As terminal thread 192 is threadingly engaged with corresponding threading extending about the fixation aperture of the implant, the comparatively shallower threading can jam on the corresponding threading extending about the fixation aperture, thereby preventing further advancement of screw 186. In other examples, the pitch of the terminal thread region 192 may be varied to cause jamming in addition to or in lieu of varying the width of the threading.

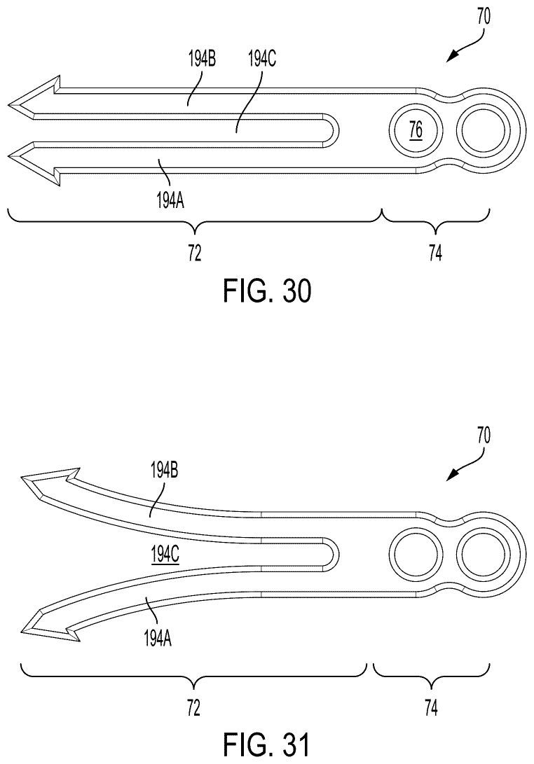

Intramedullary implant 70 has generally been described as including an implant stem 72 that may include one or more fixation apertures for receiving one or more corresponding fixation screws. FIG. 30 is top view of an alternative configuration of intramedullary implant 70 where the implant includes a stem that is configured to be deformed and enlarged in response to engaging a fixation device (e.g., screw) with the stem. As illustrated, intramedullary implant 70 includes implant stem 72 and plate 74. Plate 74 in this example includes one or more fixation apertures 76, which can be configured in various ways as described herein. Implant stem 72 is shown as being divided into a first longitudinal stem portion 194A and a second longitudinal stem portion 194B with a longitudinal gap 194C separating the two stem portions.

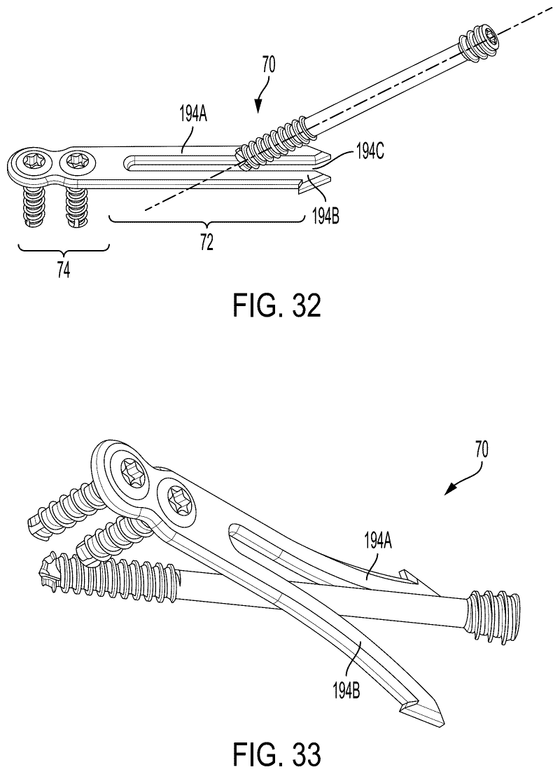

The widthwise distance of gap 194C between first longitudinal stem portion 194A and second longitudinal stem portion 194B may be set less than the diameter of a fixation member selected to be engaged with intramedullary implant stem 72. As a result, when the fixation member is inserted through gap 194C, the comparatively large size of the fixation member can cause first longitudinal stem portion 194A and second longitudinal stem portion 194B to push away from each other, thereby deforming. FIG. 31 is top view of intramedullary implant 70 from FIG. 30 illustrating an example deformation profile. FIGS. 32 and 33 illustrate an example insertion trajectory for a fixation member through the longitudinal gap in the intramedullary implant resulting in deformation of the implant stem. In some examples, the implant stem includes on or more barbs or other retaining features that engage into bone as the implant stem is deformed.

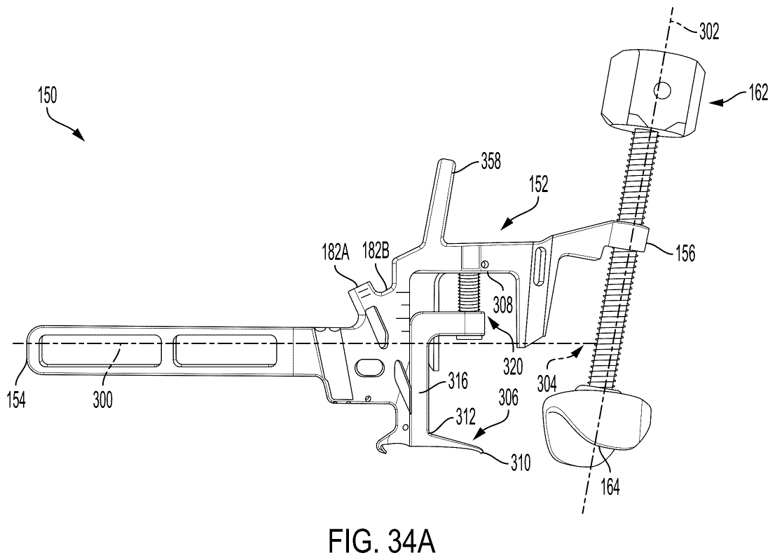

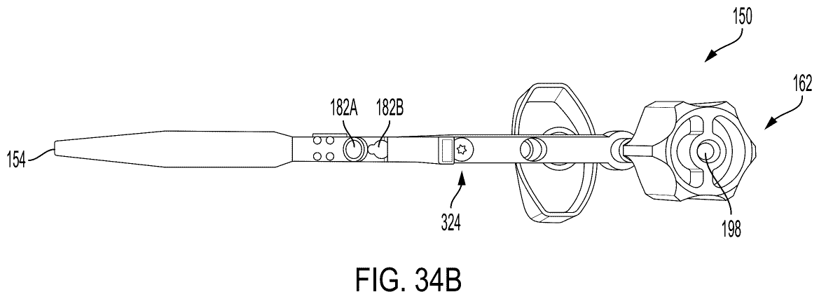

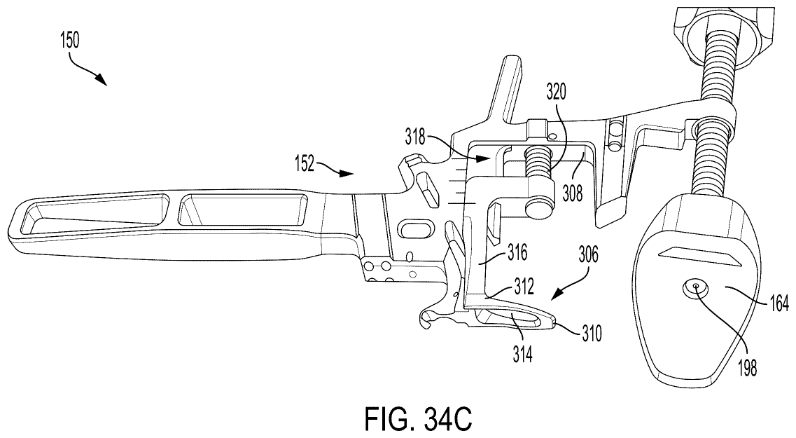

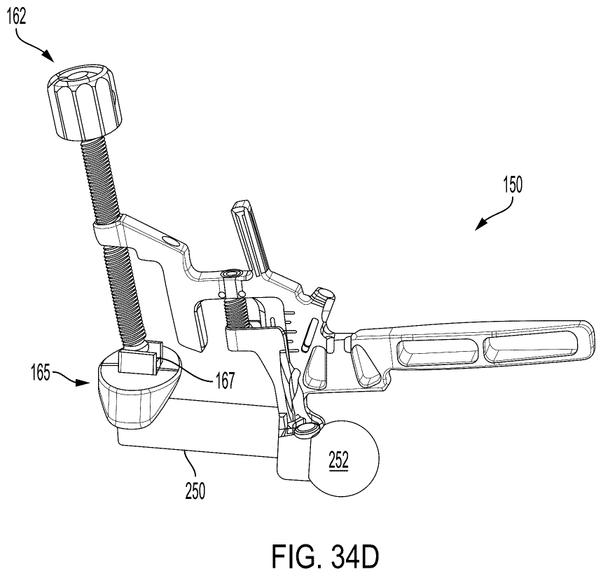

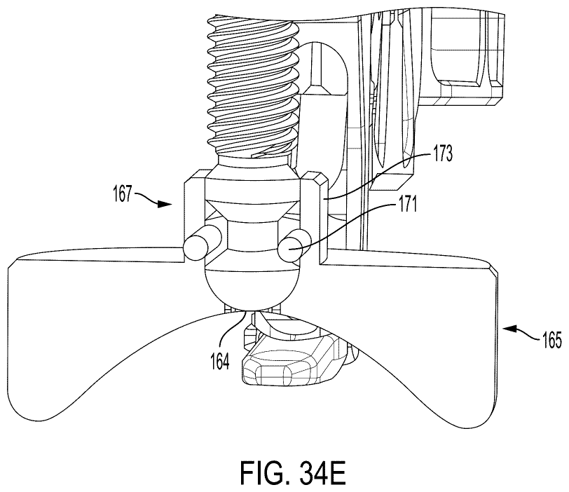

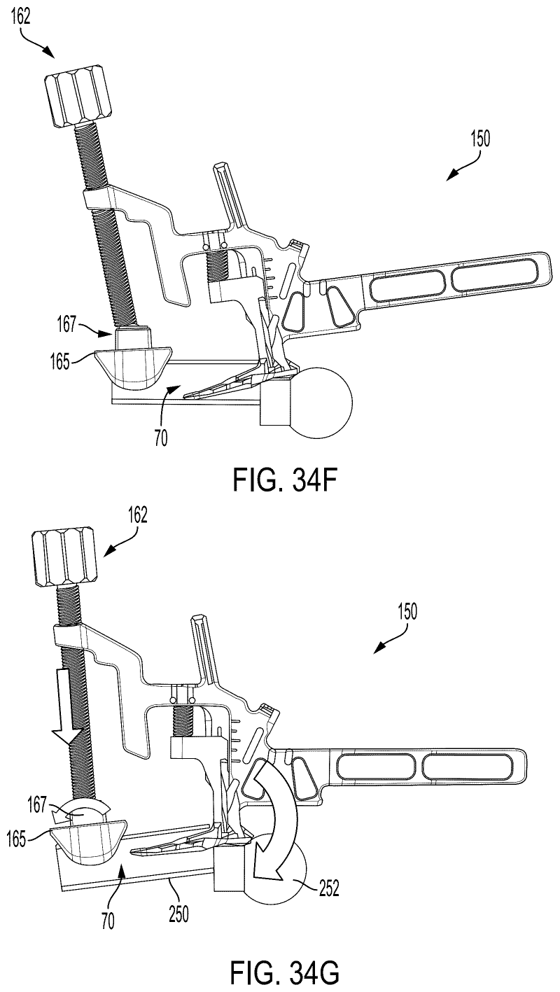

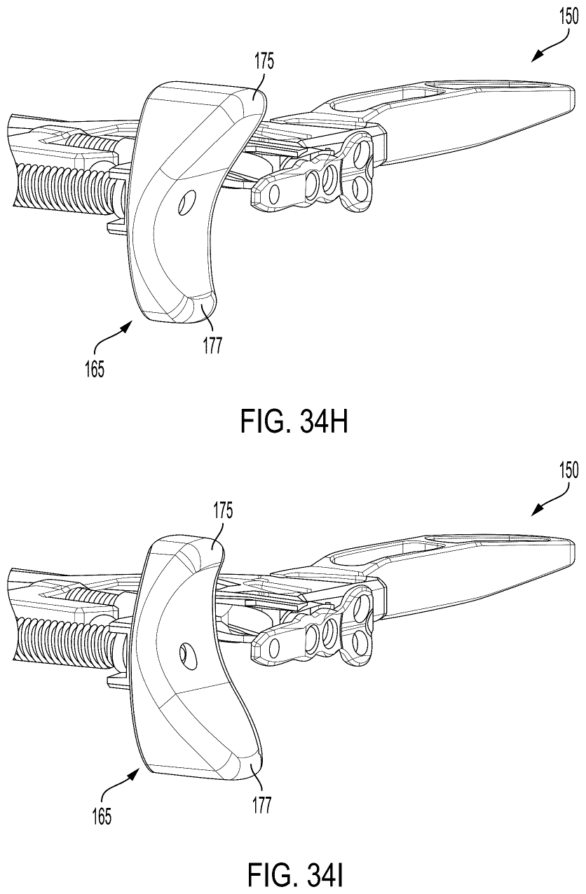

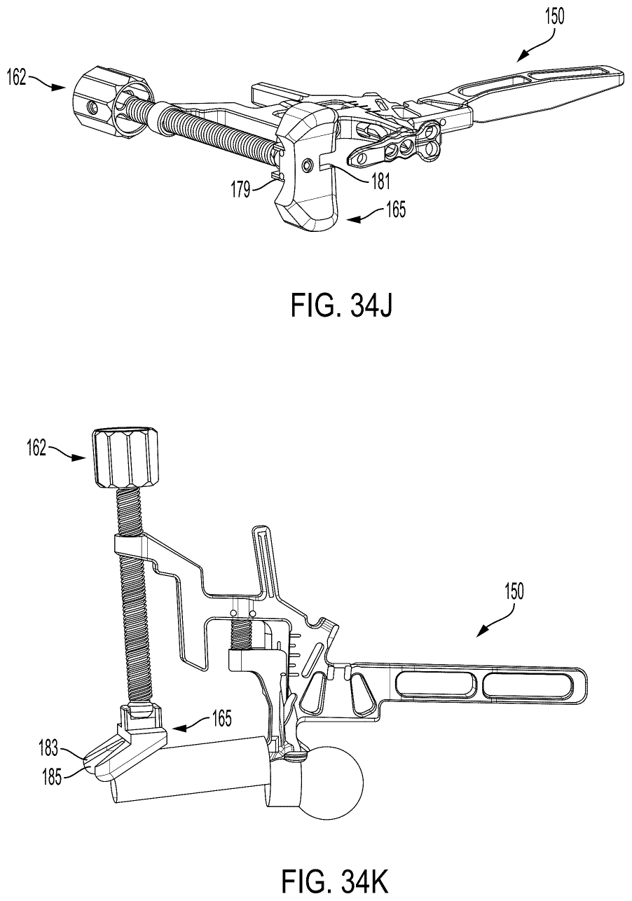

As noted above, an intramedullary implant insertion and positioning instrument according to the disclosure can have a variety of different configurations. FIGS. 34A-34C (collectively referred to as “FIG. 34”) are different views of another example configuration of instrument 150 that can be used to guide installation of an intramedullary implant 70 and/or positioning of the distal metatarsal bone portion 252 relative to the proximal metatarsal bone portion 250. FIG. 34A is a side view of instrument 150. FIG. 34B is a top view of instrument 150. FIG. 34C is a bottom perspective view of instrument 150. Reference numerals described with respect to FIGS. 22-26 refer to like features and elements in FIG. 34.

As shown in the illustrated example of FIG. 34, instrument 150 can have a body 152 extending from a first end 154 to a second end 156. Instrument 150 can include previously-described bone positioning device 162 that can apply a force between one or more bone portions contacted by the bone positioning device and the instrument. For example, bone positioning device 162 can define contact surface 164 that can contact a bone portion (e.g., directly or indirectly via an overlying skin layer that the contact surface touches). Bone positioning device 162 is illustrated in FIG. 34 as being positioned at the second end 156 of body 152 of instrument 150.

In the illustrated arrangement, bone positioning device 162 is shown as including a knob connected via a threaded shaft to body 152 of instrument 150. Body 152 can define a threaded opening through which a threaded shaft can translate. Engaging the knob of device 162 in one direction can cause the threaded shaft to advance contact surface 164 linearly away from body 152 to push the contact surface against a bone portion against which the contact surface is engaged. Engaging the knob of device 162 in the opposite direction can cause the threaded shaft to advance linearly in an opposite direction to draw contact surface 164 linearly toward body 152. Bone positioning device 162 can implemented using a variety of additional or different force delivery mechanisms, such as a sliding connection, rack and pinion, ratch connection, and/or other mechanical linkage that causes movement of one feature to apply a translational force to the structure defining contact surface 164 and/or a pin inserted therethrough.

Contact surface 164 in FIG. 34 is illustrated as being defined by a cup having a concave shape configured to conform (e.g., partially wrap about) a generally cylindrical bone portion engaged with the contact surface. The cup is illustrated positioned at the end of the threaded shaft. Threaded shaft can have a length effective to advance the cup (or other structure defining contact surface 164) colinear with and/or beyond the plane in which intramedullary implant 70 is held by instrument 150.

In some examples, bone positioning device 162 includes an opening or cannulation 198 extending through the length of the device (e.g., through the knob or other actuator, the shaft, and the cup or other contact surface). The cannulation 198 can be sized to receive a pin (e.g., K-wire) for attaching the bone positioning device to one or more underlying one portions and/or temporarily fixating a position of an underlying bone portion. In some applications, contact surface 164 of bone positioning device 162 may be offset from the underlying bone and/or skin overlying the underlying bone a distance with bone positioning device 162 being operatively engaged with the underlying bone via a pin inserted through the bone positioning device. In these applications, contact surface 164 may be referred to as a facing surface or overlying surface that faces or overlies a bone portion engaged via a wire inserted through the facing surface or overlying surface and into the underlying bone.

The shaft of bone positioning device 162 can be arranged a variety of different ways relative to body 152 of instrument 150. With respect to FIG. 34A, body 152 of instrument 150 can define a longitudinal axis 300 extending parallel to the length of the body. Bone positioning device 162 can also define a longitudinal axis 302 extending parallel to the length of the shaft of the bone positioning device. The longitudinal axis 302 of bone positioning device 162 can intersect longitudinal axis 300 of body 152 at an angle 304. Angle 304 may be a 90 degree angle or a non-90 degree angle. For example, in the illustrated arrangement, longitudinal axis 202 of bone positioning device 162 intersects longitudinal axis 300 of body 152 at an acute angle 304 such that contact surface 164 is positioned closer to first end 154 of body 152 (and, also, intramedullary implant 70 engaged with the instrument when attached) than the end of the shaft opposite the contact surface. Angling the shaft relative to the body 152 of instrument 150 (and/or lengthwise axis of intramedullary implant 70 engaged therewith) can help apply a directed force when positioning one bone portion relative to another one portion using the instrument. For example, angling the shaft relative to the body 152 of instrument 150 (and/or lengthwise axis of intramedullary implant 70 engaged therewith) can help to apply a force causing the distal metatarsal bone portion 252 to rotate and/or translate laterally (and/or while rotating and/or translating the proximal metatarsal bone portion 250 medially).

Contact surface 164 of instrument 150 can have a variety of different configurations. In some examples, contact surface 164 is defined by a body translatable relative to a shaft operatively connected to body 152 of instrument 150 or is defined by a body coupled to a shaft that is movable with the shaft. In either case, the body defining contact surface 154 can have a fixed orientation relative to the shaft or can have an adjustable orientation relative to the shaft. FIGS. 34A-34C illustrate the body defining contact surface 154 positioned at the end of a shaft with the body having a fixed orientation relative to the shaft.

FIG. 34D is a perspective view of an example configuration of instrument 150 showing the instrument configured with an adjustable body 165 defining the contact surface of the bone positioning device 162. In this example, body 165 is connected to the shaft of bone positioning device 162 via an adjustable connection 167, allowing the angular orientation of body 165 to change relative to the shaft via the adjustable connection. Configuring bone positioning device 162 with an angularly adjustable bone contact surface body can be useful for delivering a force generally perpendicular to the longitudinal axis of proximal metatarsal bone portion 250 Independent of the angular orientation of bone positioning device 162 relative to the axis of the bone.

FIG. 34E is a close-up view of body 165 defining bone contact surface 164 illustrating an example configuration for adjustable connection 167. In this example, the force provided by the shaft is transferred to body 165 via a ball and socket connection, which allows the shaft to move (e.g., via rotation of threading) yet allow the body 165 to in the same orientation and angulation relative to the foot. In this example, the shaft is coupled to body 165 through two retention pins 171 bounded by opposed walls 173. A thinned portion of the shaft allows clearance for the retention pins at different angulations. The bounding walls can be orientated to allow body 165 to rotate proximally or distally while restricting rotation plantarly or dorsally, which can cause plantar or dorsal migration of proximal metatarsal bone portion 252.

FIGS. 34F and 34G are top views illustrating example procedural steps in which instrument 150 with an angularly adjustable body 165 defining contact surface 164 is engaged with proximal metatarsal bone portion 250 and distal metatarsal bone portion 252. As shown in FIG. 34F, the stem portion of intramedullary implant 70 can be inserted into proximal metatarsal bone portion 250 at an angle through the cut face of the metatarsal bone portion. Contact surface 164 can be engaged with proximal metatarsal bone portion 250 proximally of the terminal end of the stem. Body 165 can be angled relative to the longitudinal axis of the shaft of bone positioning device 162. The clinician can engage bone positioning device 162 to adjust the alignment of the stem portion of intramedullary implant 70 from being angled relative to the longitudinal axis of proximal metatarsal bone portion 250 to being substantially parallel to the longitudinal axis of the metatarsal bone portion, as illustrated in FIG. 34G. In order for contact surface 164 defined by body 165 to continue effectively distributing the force applied by bone positioning device 162, body 165 may pivot relative to the shaft such that the body 165 remains substantially parallel to the longitudinal axis of the shaft while the angular orientation of intramedullary implant 70 and/or the shaft of bone positioning device 162 changes relative to the longitudinal axis of proximal metatarsal bone portion 250.

Body 165 defining contact surface 164 can have a variety of different configurations configured to apply a force to proximal metatarsal bone portion 250 (e.g., through the skin overlaying the metatarsal bone portion). In some examples, contact surface 164 is defined by a comparatively small cross-sectional area feature (e.g., a pin inserted percutaneously into proximal metatarsal bone portion 250 and optionally having an enlarged region pressing against the proximal metatarsal bone portion 250, such as against the skin overlying the metatarsal bone portion). In other examples, body 165 defining contact surface 164 is comparatively larger to transmit the force over a larger surface area.

In the example of FIG. 34E, body 165 is illustrated as defining a generally concave contact surface configured to contact a generally cylindrical bone, such as proximal metatarsal bone portion 250. Other example contact surface shapes include planar surfaces and V-shaped surfaces. When using a concave or V-shaped body 165, the sidewalls of the concavity or V-shaped groove may be symmetrical or asymmetrical.

In a symmetrical configuration such as illustrated in FIG. 34E, the bottom of the concavity or groove can be centered between upwardly extending sidewalls configured to receive a bone. Each sidewall can extend upwardly to the same height and/or at the same slope. In an asymmetrical configuration, one sidewall can have a different configuration than the opposing sidewall. For example, one of the sidewalls may extend upwardly from the bottom of the concavity or groove to a lower height than the opposing sidewall. An asymmetrical configuration can be useful for applying a force that is biased in one direction instead of being applied uniformly across body 165.

FIGS. 34H and 34I illustrate example configurations of body 165 defining an asymmetrical configuration in which one sidewall extends away from the bottom of the concavity or groove a shorter distance than the other sidewall. As configured, the bottom of the concavity or groove may be offset from the lengthwise and/or widthwise center of body 165. FIG. 34H illustrates an example configuration of a contact surface defining body 165 where a first side wall 175 configured to wrap at least partially about a dorsal surface of the metatarsal bone portion extends at a greater distance (e.g., laterally) then a second side wall 177 configured to wrap at least partially about a plantar surface of the metatarsal bone portion. The increased prominence of the dorsal side sidewall can act as a pivot causing the distal portion of instrument 150 to tilt plantarly. This can be beneficial to tilt the plate portion of intramedullary implant 70 carried by instrument 150 plantarly to reduce the dorsal profile of the plate portion of the implant.

FIG. 34I illustrates an alternative example configuration of a contact surface defining body 165 where the first sidewall 175 configured to wrap at least partially about the dorsal surface of the metatarsal bone portion extends a lesser distance (e.g., laterally) than the second sidewall 177 configured to wrap at least partially about a plantar surface of the metatarsal bone portion. The increased prominence of the plantar side sidewall can act as a pivot causing the distal portion of instrument 150 to tilt dorsally. This may be beneficial, e.g., when using an intramedullary implant 70 having staggered hole positions, to increase bony engagement with the plate portion of the intramedullary implant, and/or to decrease plantar prominence. In some configurations, body 165 is rotatable 180° to selectively position the elongated sidewall dorsally or plantarly depending on the needs of the clinician and the specific patient undergoing a procedure.

In the foregoing examples, the contact surface defining body 165 was generally illustrated as a solid, unbroken surface configured to generally distribute contact loading with a patient's tissue. In other examples, body 165 may define one or more grooves, openings, or other access features to allow room for insertion of wires, other targeting implements, and/or to avoid particularly regions of a patient's anatomy (e.g., a protruding bony element, sensitive tissue that may not handle being pushed for extended periods of time). FIG. 34J illustrates an example configuration of a contact surface defining body 165 for bone positioning device 162 having an example side edge cutout or chamfer 179 (e.g., allowing for broad clearance of anatomy) and/or an example slot or opening 181 extending through the thickness of the body (e.g., for passing discrete elements, such as a screw and/or wire).

FIG. 34K illustrates another example configuration of a contact surface defining body 165 for bone positioning device 162 defining one or more pointed contact surfaces. In this case, body 165 may include one or more (e.g., two) prominent prongs 183 that can engage the bone or tissue through a point contact, e.g., providing a rigid connection (as compared to general abutment) with the underlying anatomy. The prongs 183 may include a gap 185 between the prongs to cause the prongs to be more prominent and/or to allow passing of other instruments (e.g., a burr, wires for temporary fixation), and/or other implants (e.g., staple, screw).

With further reference to FIGS. 34A-34C, instrument 150 is shown as including an intramedullary insertion body 306 operatively connected to body 152. Intramedullary insertion body 306 can be inserted into the medullary cannel of a bone portion (e.g., proximal metatarsal bone portion 250) concurrent with intramedullary implant 70. Intramedullary insertion body 306 can move relative to instrument body 152 to apply a positioning force between body 152 of instrument 150 and proximal metatarsal bone portion 250 and/or distal metatarsal bone portion 252. A clinician can translate intramedullary insertion body 306 to pull or push a bone portion engaged with the intramedullary insertion body in a direction of movement desired by the clinician. For example, during initial set up, intramedullary insertion body 306 may be positioned offset from a support surface 308 of body 152 a maximum offset distance. During subsequent use, a clinician may cause intramedullary insertion body 306 to move closer toward support surface 308 of body 152, pulling the proximal metatarsal bone portion 250 away (e.g., medially outwardly) from the distal metatarsal bone portion 252.

Intramedullary insertion body 306 can have a variety of different configurations. In the illustrated example, intramedullary insertion body 306 defines a length extending from a first end 310 to a second end 312. Intramedullary insertion body 306 is insertable into a medullary canal of a bone portion, e.g., by inserting first end 310 through a cut end face of a metatarsal bone portion and advancing the body into the medullary canal of the bone portion. In some examples, the thickness of body 206 tapers from second end 312 to first end 310, e.g., such that the body is thicker adjacent second end 312 than at first end 310. This can provide a body with a tapered profile to help insert the body into the medullary canal.

In different implementations, intramedullary insertion body 306 may be a solid structure devoid of openings or may, instead, include one or more openings extending through the thickness of the body. For example, as seen in FIG. 34C, intramedullary insertion body 306 may include an opening 314 extending through the thickness of the body. Opening 314 may be sized to receive a drill and/or screw that can be inserted therethrough. During use, intramedullary implant 70 can be engaged with instrument 150 adjacent to and/or in contact with intramedullary insertion body 306. Opening 314 may provide access through intramedullary insertion body 306 to one or more apertures of intramedullary stem portion 72 of intramedullary implant 70. During use, a clinician may drill a screw hole and/or insert a screw through opening 314 and into the underlying aperture(s) of intramedullary stem 72.

Intramedullary insertion body 306 can be operatively connected to body 152 of instrument 150. For example, intramedullary insertion body 306 may be connected to and/or extend from a support arm 316 (optionally defining a unitary structure) operatively connected to body 152. In use, intramedullary insertion body 306 can be positioned in a medullary canal of a bone while support arm 316 resides outside of the medullary canal, e.g., such as extending transversely to a cut end face of a bone into which intramedullary insertion body 306 is inserted. Support arm 316 can be slidably connected to body 152 via a sliding connection 318 between the support arm and body.

Instrument 150 may include an intramedullary insertion body translation mechanism 320 for controlling translational movement of the intramedullary insertion body relative to body 152. In the illustrated arrangement, mechanism 320 is shown as a threaded shaft connected to intramedullary insertion body 306 on one end and connected to body 152 on an opposite end. The threaded shaft can include a drive receptacle 324 (FIG. 34B) configured to receive a driver (e.g., hexalobular driver) through which a rotational force can be applied to turn the shaft. In some examples, a knob is provided on the end of the threaded shaft through which a rotational force can be applied to control positioning of intramedullary insertion body 306. In either case, turning the threaded shaft one direction can cause the shaft to pull intramedullary insertion body 306 toward support surface 308. During the threaded shaft in the opposite direction can cause the threaded shaft to push intramedullary insertion body 306 away from support surface 308. While instrument 150 illustrates intramedullary insertion body translation mechanism 320 as comprising a threaded shaft, other mechanical linkages can be used that causes movement of one feature to apply a translational force to intramedullary insertion body 306, including those discussed above.

Intramedullary insertion body 306 can be permanently connected to body 152 of instrument 150 (such that the intramedullary insertion body is not designed or configured to be removed from instrument 150). Alternatively, intramedullary insertion body 306 may be detachably connected to body 152 of instrument 150. Detachably connecting intramedullary insertion body 306 to instrument 150 can be beneficial to allow the intramedullary insertion body to be removed, for example for cleaning, repair, and/or replacement. In some applications, a system of different intramedullary insertion bodies 306 are provided where each intramedullary insertion body in the system varies from each other intramedullary insertion body in the system by one or more characteristics (e.g., size, shape, angulation, the presence or configuration of one or more openings extending through the body, the material of construction).

While instrument 150 is generally described as being configured so intramedullary implant 70 is positioned at a fixed location relative to body 152 of the instrument and intramedullary insertion body 306 moves relative to body 152, in other examples, intramedullary implant 70 may be movable relative to intramedullary insertion body 306. In these examples, intramedullary insertion body 306 may be fixed relative to body 152 of instrument 150 or may also be movable relative to the body. Thus, it should be appreciated that discussion of intramedullary insertion body 306 being movable relative to body 152 may additionally or alternatively be implemented by configuring intramedullary implant 70 to be movable relative to intramedullary insertion body 306. Accordingly, instrument 150 can be operatable to provide relative movement between intramedullary insertion body 306 and intramedullary implant 70 to controllably adjust the distance and spacing between the components, whether intramedullary implant 70 is configured to move, intramedullary insertion body 306 is configured to move, or both intramedullary implant 70 and intramedullary insertion body 306 are configured to move.

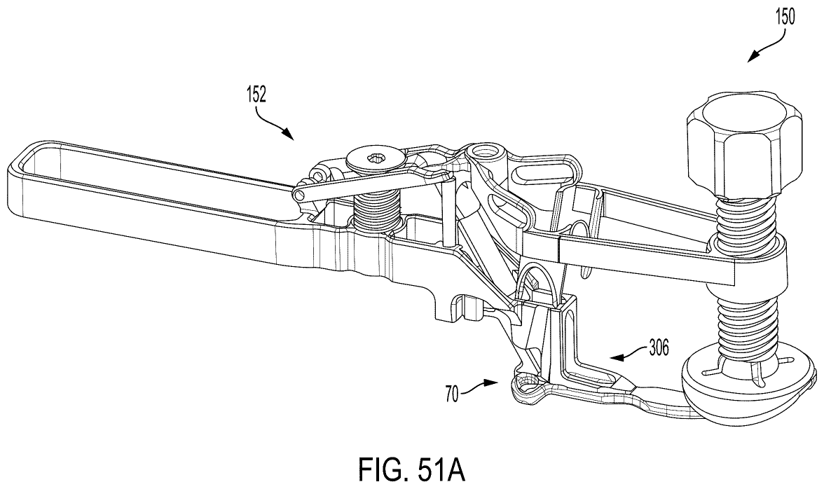

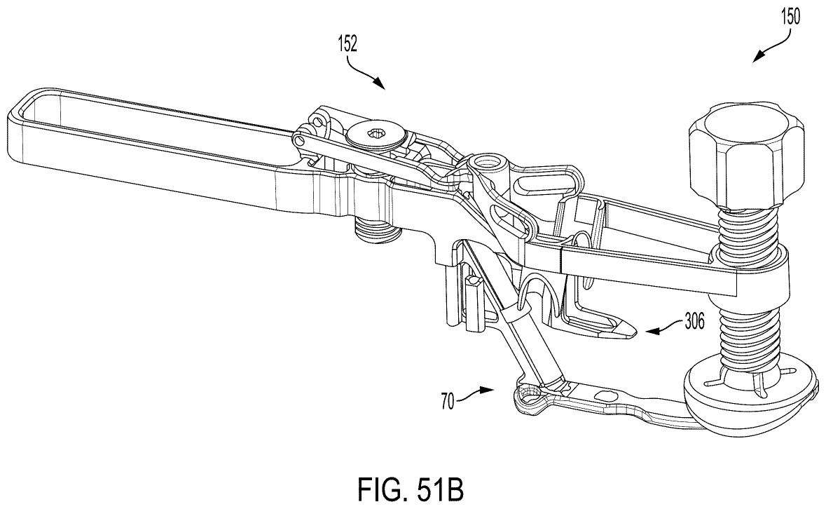

FIGS. 51A and 51B are illustrations of an example configuration of instrument 150 in which intramedullary implant 70 is operatively connected to body 152 and movable relative to the body. In these examples, intramedullary insertion body 306 is illustrated as being fixedly positioned relative to the body and intramedullary implant 70 is releasably and movably coupled to body 152. For example, intramedullary implant 70 may be releasably coupled to a movable section of body 152 using any of the techniques described herein. The movable section of body 152 can move (e.g., translate linearly) relative to a stationary section of body 152 (to which intramedullary insertion body 306 is coupled) to provide relative movement between intramedullary implant 70 (when coupled to the movable section of the body) and intramedullary insertion body 306. In the illustrated example, the movable section of body 152 is illustrated as being configured to linearly translate via control of a mechanical linkage, which can be implemented using any of the example mechanical linkages discussed herein. In some examples, both intramedullary implant 70 and intramedullary insertion body 306 are movable relative to a stationary section of body 152, e.g., combining embodiments of instruments 150 discussed herein.

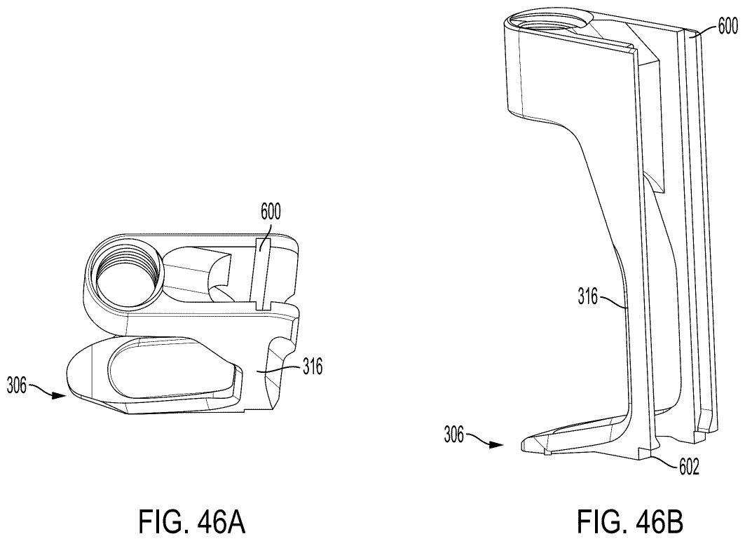

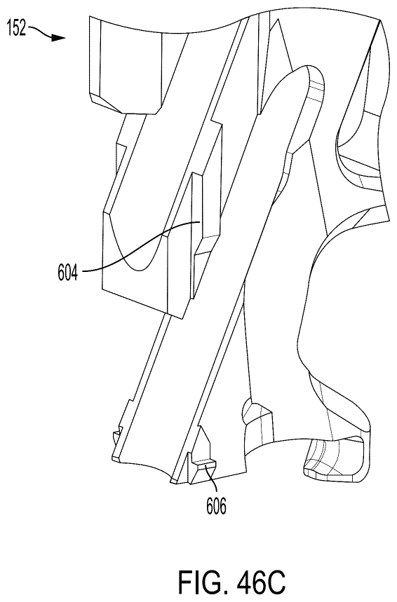

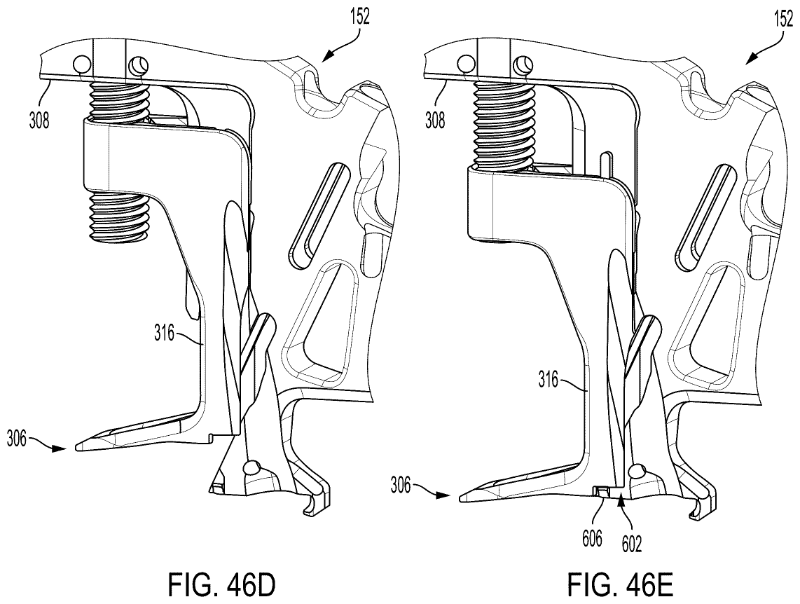

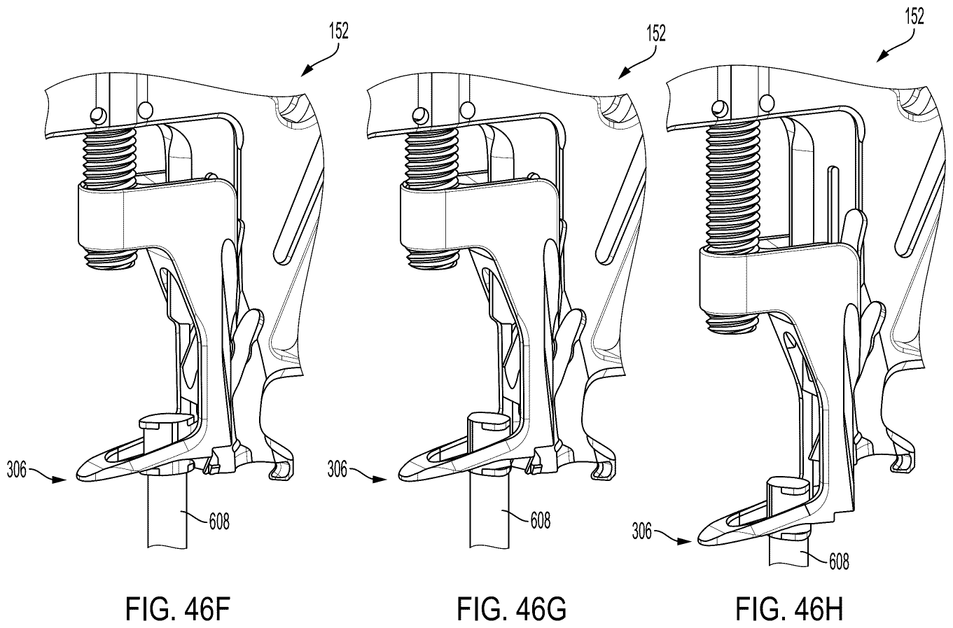

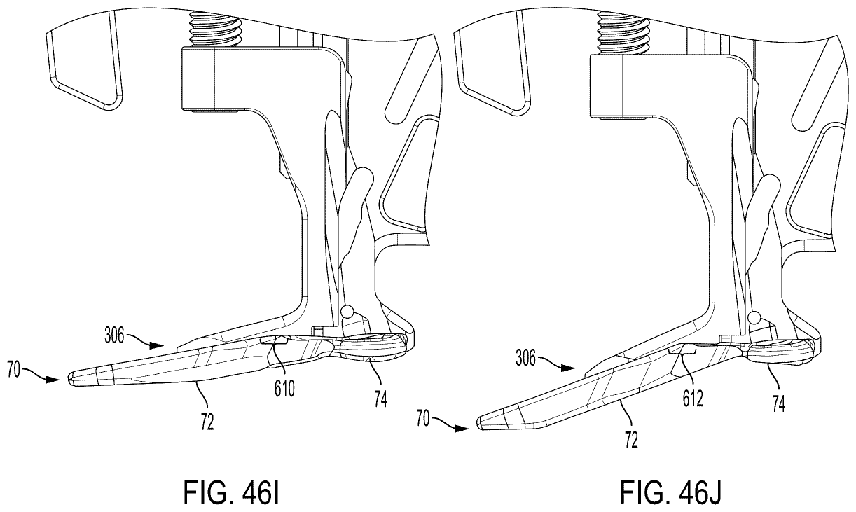

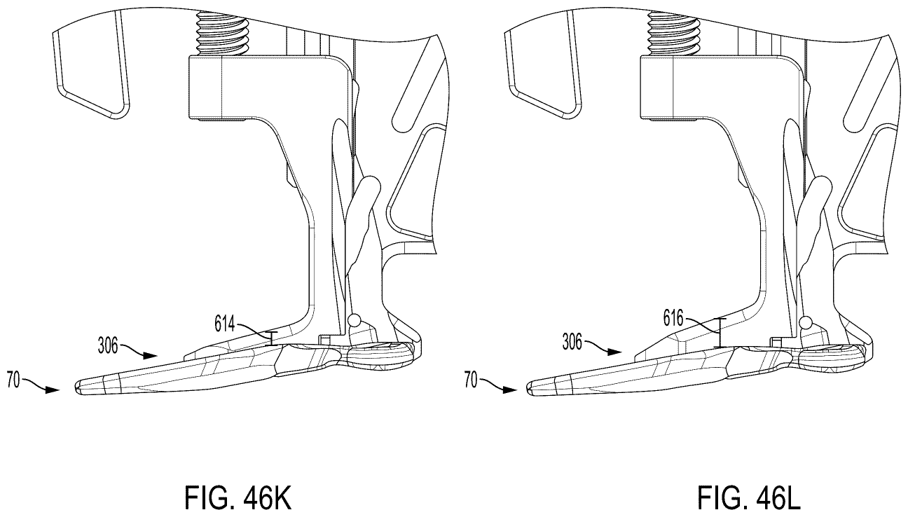

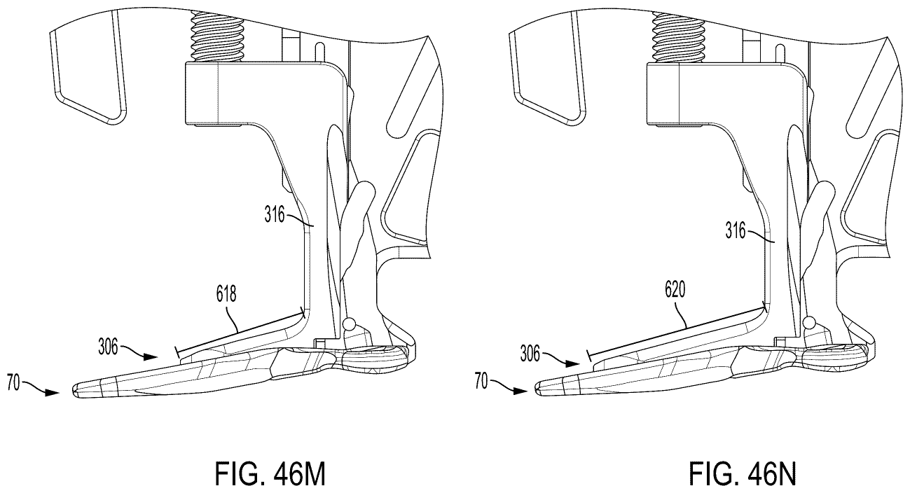

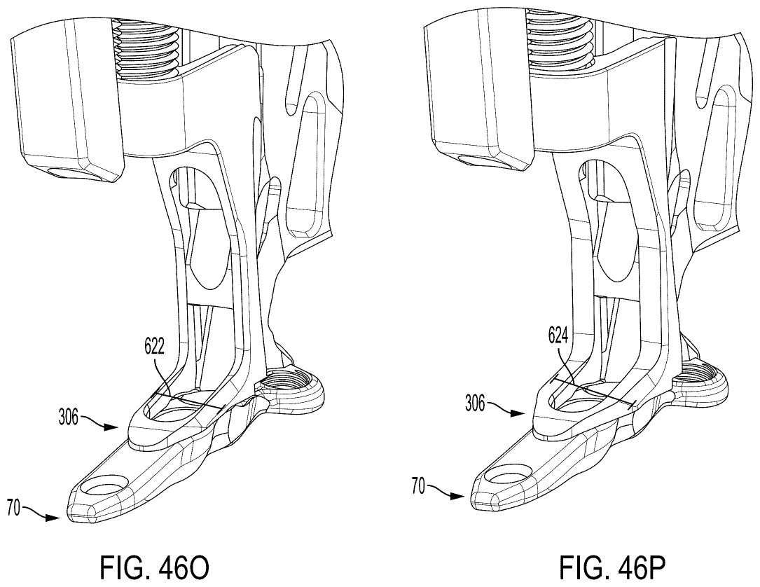

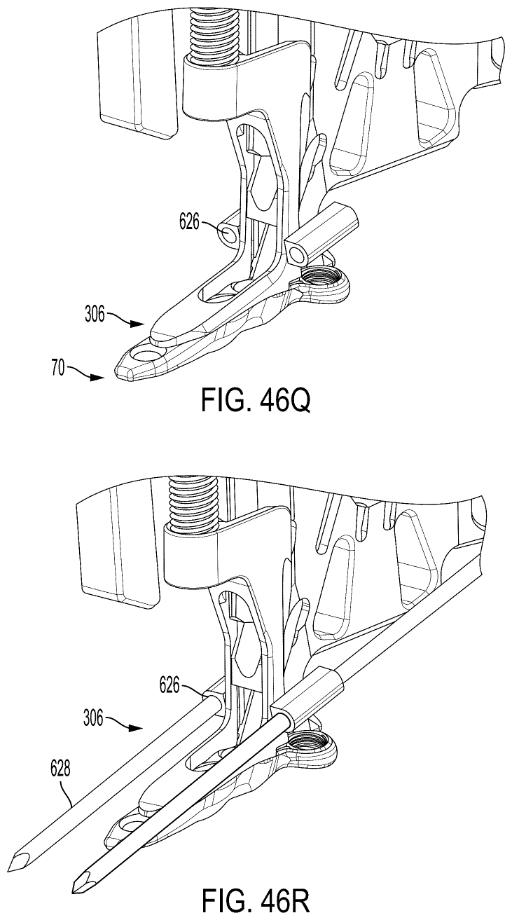

FIGS. 46A and 46B is an angled top view and a partially sectionalized perspective view, respectively, of an example assembly that includes a support arm 316 connected to and/or extending from intramedullary insertion body 306. As shown in these figures, the assembly may include one or more male or female connection features 600 that are configured to engage with one or more corresponding female or male connection features on body 152 of instrument 150. FIGS. 46A and 46B particulate illustrate example configuration having a pair of opposed grooves 600 that receive corresponding projections of body 152 of instrument 150 to define a sliding connection there between. The grooves may terminate at a shelf 602 that delimits the extent to which the assembly can slide relative to body 152 of instrument 150. For example, FIG. 46C as a partial sectionalized view of an example configuration of body 152 of instrument 150 defining male projection features 604 that can be received in grooves 600. A terminal tab 606 can be formed collinear with projection features 604 to restrict the movement and delimit the extent to which the assembly that includes arm 316 and intramedullary insertion body 306 can move relative to body 152 of instrument 150.

FIGS. 46D and 46E are side views of an example intramedullary insertion body assembly showing different positions of the assembly relative to the body 152 of the instrument. FIG. 46D shows the assembly partially retracted toward support surface 308. FIG. 46E shows the assembly advanced to the maximum offset distance from support surface 308. As shown in this position, tabs 606 of body 152 are received in the space defined by shelf 602 and contact the edge surface of the assembly, preventing the assembly from being advanced further away from support surface 308.

To detach the intramedullary insertion body assembly from body 152 of instrument 150, a wedge or cam can be inserted into an opening extending through intramedullary insertion body 306 and a force applied to push the rails of the assembly apart so can be removed from body 152. FIGS. 46F-46H our different illustrations showing an example cam device 608 inserted through an opening in intramedullary insertion body 306 and being turned to spread the legs of the assembly apart. With the legs of the intramedullary insertion body assembly spread apart, the assembly can be advanced along the coupling shaft until the assembly is released from the terminal end of the coupling shaft.

With intramedullary insertion body 306 removed from instrument 150, the same or a different intramedullary insertion body may be coupled to body 152 of the instrument. As mentioned, a system that includes two or more different intramedullary insertion bodies 306 (e.g., two, three, four, five or more) can be provided to provide procedural flexibility using instrument 150. As one example, intramedullary insertion bodies having different angles may be provided. FIG. 46I illustrates an example intramedullary insertion body 306 configured to contact and/or engage with an example intramedullary implant 70 defining a first intersection angle 610 between the stem portion 72 of the implant and the plate portion 74 of the implant. FIG. 46J illustrates a different example intramedullary insertion body 306 configured to contact and/or engage with a different example intramedullary implant defining a second intersection angle 612 between the stem portion 72 of the implant and the plate portion 74 of the implant. Intramedullary insertion body 306 can be changed depending on the type and configuration of intramedullary implant 70 desired to be used for a particular procedure, e.g., such that the intramedullary insertion body has a shape corresponding to the shape of the selected intramedullary implant.

As another example, intramedullary insertion bodies having different heights may be provided. FIG. 46K illustrates an example intramedullary insertion body 306 have an example height 614 (e.g., measured midway along the length of the insertion body). FIG. 46L illustrates a different example intramedullary insertion body 306 have a different example height 616 (e.g., also measured midway along the length of the insertion body). Providing intramedullary insertion bodies of different heights may be useful to control the amount of initial distraction of proximal metatarsal bone portion 250 relative to distal metatarsal bone portion 252 upon insertion of the intramedullary insertion body and intramedullary implant stem portion through the cut end face of the proximal metatarsal bone portion.

As another example, intramedullary insertion bodies having different lengths may be provided. FIG. 46M illustrates an example intramedullary insertion body 306 have an example length 618 (e.g., measured from the face of support arm 316 to the terminal end of the insertion body). FIG. 46N illustrates a different example intramedullary insertion body 306 have a different example length 620 (e.g., also measured the face of support arm 316 to the terminal end of the insertion body). Providing intramedullary insertion bodies of different lengths may be useful depending on the length of the metatarsal of the particular patent undergoing a procedure and/or if extended distribution of loading forces across the medial cortex is desired (e.g., in instances of poorer bone quality and/or if a portion of the medial cortex has been violated).

As another example, intramedullary insertion bodies having different widths may be provided. FIG. 46O illustrates an example intramedullary insertion body 306 have an example width 622. FIG. 46P illustrates a different example intramedullary insertion body 306 have a different example width 624. Providing intramedullary insertion bodies of different widths may be useful depending on the size of the patient's anatomy and/or to control the distribution of loading forces across the medial cortex.

As another example, intramedullary insertion bodies having supplemental features, such as wire receiving apertures, may be provided. FIG. 46Q illustrates an example intramedullary insertion body 306 have wire receiving apertures 626. FIG. 46R illustrates the intramedullary insertion body 306 with wires 628 inserted through the wire receiving apertures 626. Configuring intramedullary insertion body 306 with wire receiving apertures 626 may be useful if the medial cortex of proximal metatarsal bone portion 250 breaks during insertion and/or application of force using a first intramedullary insertion body without wire receiving apertures. The initial intramedullary insertion body can be replaced with the intramedullary insertion body 306 with wire receiving apertures 626 functioning as a rescue component. The wire receiving apertures 626 can allow the clinician to insert wires 628 down the medial cortex to provide structural support and/or to use as an anchor to translate against. The wires 628 may be inserted parallel to the medial cortex or can be angled to be converging, diverging, and/or staggered.

With further reference to FIG. 34, instrument 150 (as with instrument 150 in FIG. 22) can also define one or more screw insertion apertures that are coaxially aligned one or more of a corresponding fixation apertures of intramedullary implant 70 (when the intramedullary implant is attached to the instrument). For example, with reference to FIG. 34B, body 152 of instrument 150 may define one or more screw insertion apertures 182 (e.g., apertures 182A, 182B) aligned with the trajectory of the one or more fixation apertures 88 extending through intramedullary implant stem 72 (e.g., fixation apertures 88A, 88B extending through implant stem 72 as illustrated in FIGS. 13D and 13E). The number and arrangement of screw insertion apertures defined by body 152 may vary depending on the number and arrangement of screw apertures defined by intramedullary implant 70.

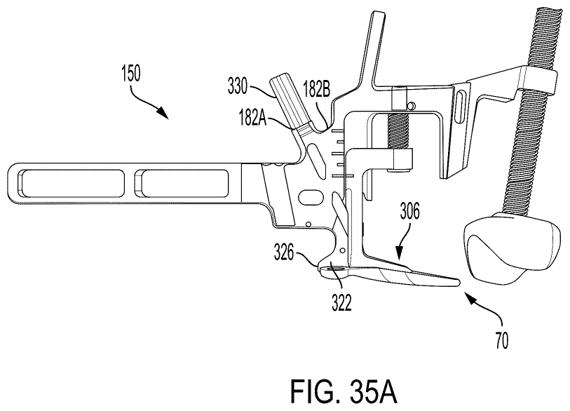

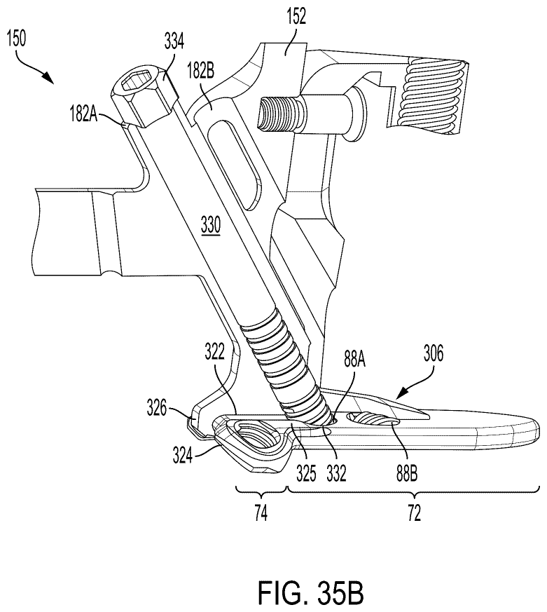

Instrument 150 can receive and hold intramedullary implant 70 to facilitate positioning of the implant relative to proximal metatarsal bone portion 250 and/or distal metatarsal bone portion 252 using the instrument. Instrument 150 can have a variety of different engagement configurations that function to releasably engage intramedullary implant 70. FIG. 35A is a side view of instrument 150 illustrating an example implant attachment arrangement that can be used to attach intramedullary implant 70 to the instrument. FIG. 35B is a sectional side view of a portion of instrument 150 further illustrating example attachment features that can be used to attach intramedullary implant 70 to the instrument.

In the example of FIGS. 35A and 35B, instrument 150 is illustrated as defining an implant engagement surface 322 that is configured to be positioned adjacent to and/or in contact with a surface intramedullary implant 70. For example, plate portion 74 of intramedullary implant 70 can define a bone contacting surface 324 (FIG. 35B) and a facing surface 325 opposite the bone contacting surface. Bone contacting surface 324 of plate portion 74 of intramedullary implant 70 may be positioned in contact with distal metatarsal bone portion 352 bone attachment of the intramedullary implant. At least a portion of bone contacting surface 324 of plate portion 74 can be brought into contact with implant engagement surface 322 of instrument 150.

In some configurations, instrument 150 includes a hook 326 configured to wrap at least partially about the thickness of intramedullary implant 70. For example, hook 326 can extend from implant engagement surface 320 across an edge of plate portion 74 defined by the thickness of the plate of portion and, in some examples, extend at least partially under and/or in contact with bone engagement surface 324. In configurations in which intramedullary implant 70 includes a distal recesses 81 (e.g., FIGS. 13D and 13E), hook 326 may be configured (e.g., sized and positioned) to be received in the distal recesses 81 and to capture intramedullary implant 70 at that location. When so configured, at least a portion of one or more lobes defining apertures extending through plate portion 74 of intramedullary implant 70 may extend distally beyond the proximal-most surface of hook 326, where the hook contacts the implant. In other configurations, instrument 150 may hold intramedullary implant 70 relative to contact surface 322 defined by body 152 without utilizing a hook wrapping partially or fully about a thickness of the implant.

In either case, intramedullary implant 70 may be mechanically fixated to instrument 150 to temporarily hold the implant relative to the instrument until desirably released. In the illustrated arrangement, an attachment rod 330 is used to help secure intramedullary implant 70 to instrument 150. Attachment rod 330 can extend from a first end 332 engageable with a threaded aperture of intramedullary implant 70 to a second end 334 secured relative to body 152 of instrument 150. In some implementations, intramedullary implant 70 is configured to be attached instrument 150 by inserting attachment rod 330 through a screw insertion aperture 182A extending through the body 152 of the instrument. Screw insertion aperture 182A can be aligned with the trajectory of a fixation apertures 88A extending through intramedullary implant stem 72.

A region of attachment rod 330 adjacent first end 332 can be threadingly engaged with a fixation aperture 88A of intramedullary implant 70. A region of attachment rod 330 adjacent second end 334 can be enlarged relative to screw insertion aperture 182A to function as a depth limiter that limits the depth to which the rod can be inserted into the screw insertion aperture. Attachment rod 330 may have a length effective to threadingly engage fixation aperture 88A without extending beyond the thickness of intramedullary implant 70 while simultaneously abutting body 152 adjacent second end 334. In various examples, second end 334 of attachment rod 330 may include a knob and/or define a driver receiving cavity configured to receive a driver. In either case, a clinician can interact with attachment rod 330 to engage and/or disengage the rod from the intramedullary implant by rotating the rod into and/or out of the fixation aperture of the implant. In some examples, attachment rod 330 may be cannulated to allow a K-wire, drill bit, and/or another elongate instrument to be advanced through the cannulation of the rod. Other mechanical connections can be used to releasably couple intramedullary implant 70 to instrument 150.

As noted, instrument 150 can define one or more screw insertion apertures that are coaxially aligned one or more of a corresponding fixation apertures of intramedullary implant 70 (when the intramedullary implant is attached to the instrument). The clinician can then use the one or more screw insertion apertures to drill pilot holes and/or guide fixation screws through each screw insertion aperture of instrument 150 (and correspondingly through underlying fixation apertures of intramedullary implant 70 while the implant remains engaged with the instrument). This can address the challenge the clinician may otherwise face of accurately locating each fixation aperture of intramedullary implant 70 after inserting the intramedullary implant into proximal metatarsal bone portion 250.

In the illustrated example of FIGS. 34, 35A, and 35B, instrument 150 is illustrated as defining screw insertion apertures 182A and 182B aligned fixation apertures 88A and 88B, respectively, extending through intramedullary stem 72. In this arrangement, fixation apertures 88A and 88B are substantially centered across the width of intramedullary implant 70 and spaced from each other lengthwise across the implant. Instrument 150 may include fewer (e.g., one) or more (e.g., three or more) screw insertion apertures corresponding to each fixation aperture of intramedullary implant 70 that is substantially centered across the width of intramedullary implant (e.g., on intramedullary stem portion 72 and/or plate 74 of the implant). Instrument 150 may additionally or alternatively include one or more screw insertion apertures corresponding to one or more fixation apertures of intramedullary implant 70 located offset from a longitudinally bisecting centerline of the implant. To limit the thickness of body 152 of instrument 150 in this situations (e.g., for easier manipulation by the clinician and better visualization under fluoroscopic imaging), instrument 150 may include a detachable guide that defines one or more screw insertion apertures and is detachably couplable to body 152 of the instrument.

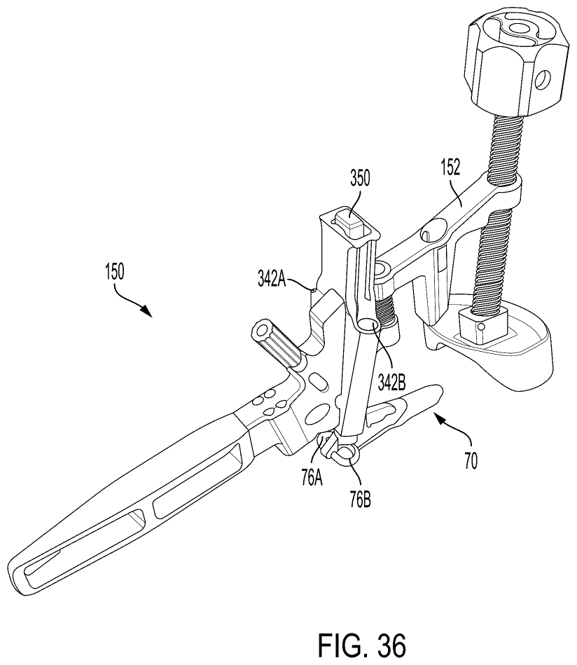

FIG. 36 is a perspective view of instrument 150 illustrating an example detachable guide body 340 that can be detachably connected to body 152 of instrument 150. Guide body 340 can define one or more screw insertion apertures 342 (e.g., apertures 342A, 342B) aligned with the trajectory of the one or more fixation apertures 76 extending through plate 74 (e.g., fixation apertures 76A, 76B extending through plate 74 as illustrated in FIGS. 13D and 13E) and/or intramedullary stem portion 72 of the implant. The number and arrangement of screw insertion apertures defined by guide body 340 may vary depending on the number and arrangement of screw apertures defined by intramedullary implant 70. The one or more screw insertion apertures 342 of guide body 340 may be configured to be positioned over one or more fixation apertures extending through intramedullary implant 70 (e.g., intramedullary stem portion 72 and/or plate 74 of the implant) that are not substantially centered across the width of intramedullary implant (e.g., the geometric center of the fixation aperture is offset from a longitudinally bisecting centerline of the implant).

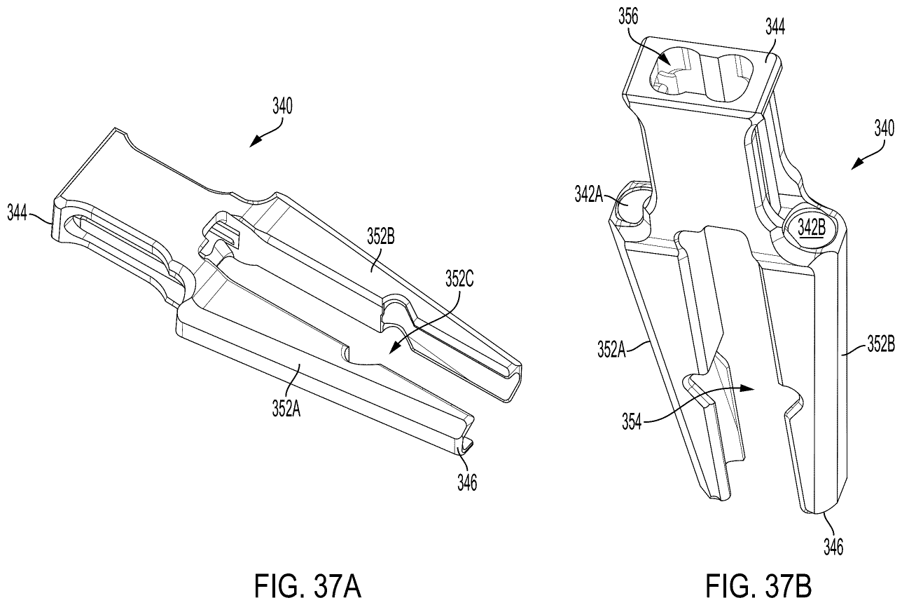

FIGS. 37A and 37B are different perspective views of an example configuration of detachable guide body 340 shown separated from body 152 of instrument 150. In this example, guide body 340 defines a structure extending from a first end 346 to a second end 350. Guide body 340 is divided into a pair of arms 352A and 352B separated by a space 354 between the two arms. Each arm defines a corresponding screw insertion apertures 342A, 342B (which may also be referred to as a guide aperture or drill aperture), which may be a partially or fully bounded channel extending at least partially along the length of guide body 340. Each screw insertion aperture 342A, 342B may be configured (e.g., sized, shaped, positioned) such that, when guide body 340 is attached to body 152 of instrument 150, the screw insertion aperture is positioned co-axially with and overlying a corresponding aperture of intramedullary implant 70. For example, when intramedullary implant 70 includes a pair of lobes defining apertures 76A, 76B offset in side-by-side arrangement from the lengthwise centerline of the implant (e.g., such as illustrated in FIGS. 13D and 13E), screw insertion aperture 342A, 342B may be configured be aligned with and positioned over the apertures defined by the lobes.

Guide body 340 can be removably connected to body 152 of instrument 150. Guide body 340 in main body 152 can have a variety of different complementary connection features (e.g., corresponding male and female connectors) to facilitate interconnection of the components. In the illustrated arrangement, guide body 340 defines a center connection aperture 356 that is co-axially aligned with space 354 separating arms 352A and 352B. With reference to FIG. 36A, body 152 can have a male projection 358 configured to be inserted into connection aperture 356 of guide body 340.

To connect guide body 340 to body 152, the guide body can be positioned with arms 352A and 352B on opposite widthwise sides of body 152 and the guide body slidingly advanced over the guide body. Arms 352A, 352B can be separated from each other a distance sufficient to position the arms on opposite sides of body 152. Arms 352A, 352B may exhibit some flexing such that the second end 346 of the arms can flex outwardly as guide body 340 is engaged with body 152. The arms can then bias inwardly once guide body 340 is engaged with body 152 to help frictionally retain the guide body 340 to the main body 152. In either case, as guide body 340 is advanced onto body 152, projection 358 of body 152 can be received into connection aperture 356 of guide body 340 and advanced partially or fully through the aperture.

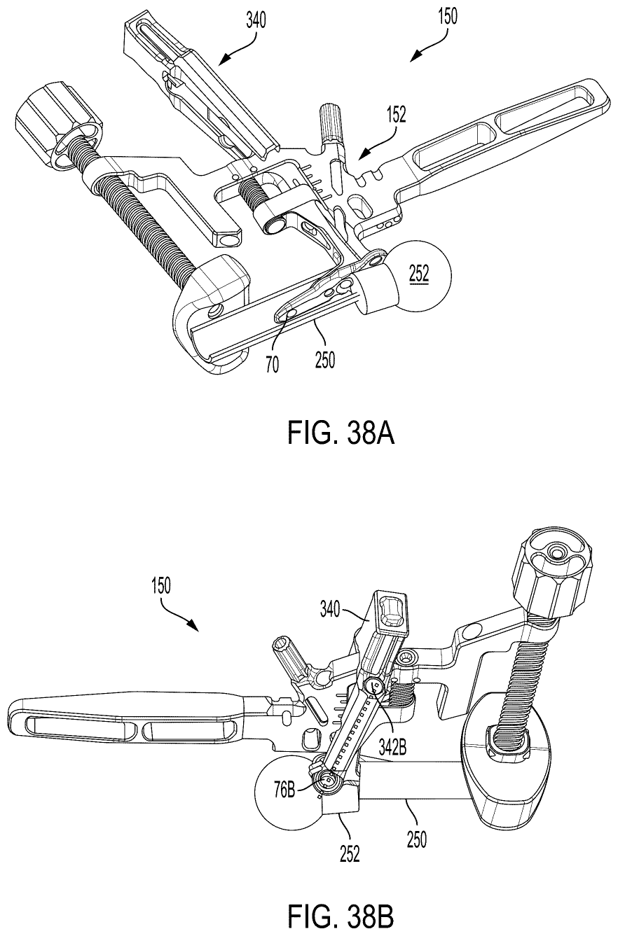

FIG. 36 illustrates guide body 340 fully seated on body 152 with screw insertion aperture 342A, 342B positioned co-axially with and overlying apertures 76A, 76B of intramedullary implant 70. The second end 346 of guide body 340 is shown offset from intramedullary implant 70 such that there is a space between the guide body and the implant. In other configurations, guide body 340 may have a length effective to contact intramedullary implant 70 when the guide body engaged with the main body 152. FIG. 38A illustrates guide body 340 being inserted onto main body 152 of instrument 150. FIG. 38B illustrates guide body 340 seated on and operatively engaged with body 152.

In use, a clinician can use screw insertion aperture 342A, 342B to guide insertion of screws through apertures 76A, 76B and into an underlying bone portion. The clinician may use screw insertion aperture 342A, 342B to guide a drill bit to drill holes through the apertures of intramedullary implant 70 into the underlying bone. Thereafter, the clinician can advance screws through the apertures of the implant and into the underlying bone via the predrilled holes, e.g., advancing the screws until the screws are fully seated in the implant. In some examples, the clinician advances the screws through screw insertion aperture 342A, 342B of guide body 340. In other examples, the clinician may use screw insertion aperture 342A, 342B to guide a drill bit and/or insertion of a K-wire through each aperture and then remove guide body 340 before inserting the screws through a drill hole and/or along a K-wire guided using guide body 340.

Instrument 150 includes a bone positioning device 162 operable to apply a force to move distal metatarsal bone portion 252 relative to proximal metatarsal bone portion 250 during a bone realignment procedure, such as a bunion correction procedure. Engagement of bone positioning device 162 can cause the distal metatarsal bone portion 252 to move laterally in the transverse plane and/or proximal metatarsal bone portion 250 to move immediately in the transverse plane. For example, engagement of bone positioning device 162 can apply a force that causes distal metatarsal bone portion 252 to move laterally in the transverse plane, increasing the offset between the cut end faces of the two bone portions. After fixation using intramedullary implant 70, bone growth can fuse the two bone portions together in a corrected alignment and fill the offset between the cut end faces the two bone portions.

While bone positioning device 162 can apply a force to move a bone portion in the transverse plane, the clinician may additionally or alternatively desire to move the bone portion in one or more other planes, such as the frontal plane and/or sagittal plane. To move the bone portion in the sagittal plane, the clinician may grasp the bone portion (optionally via a K-wire inserted into the bone portion) and dorsiflex or plantar flex the bone portion. Additionally or alternatively, the clinician may grasp the bone portion (optionally via a K-wire inserted into the bone portion) and rotate the bone portion in the frontal plane.

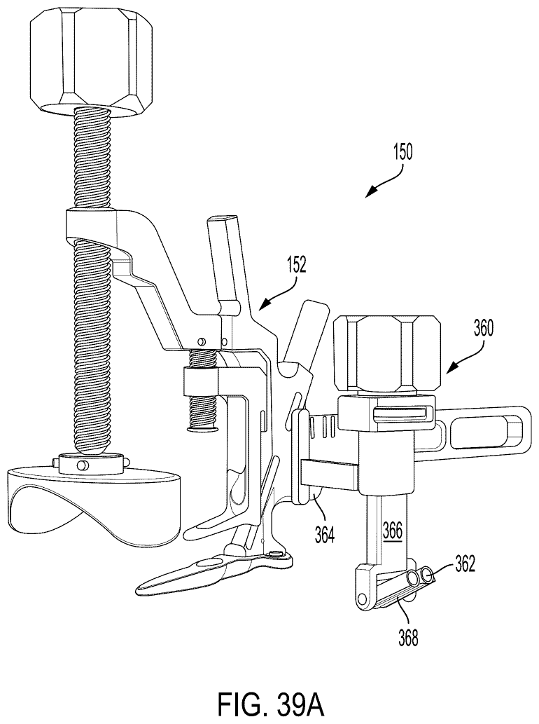

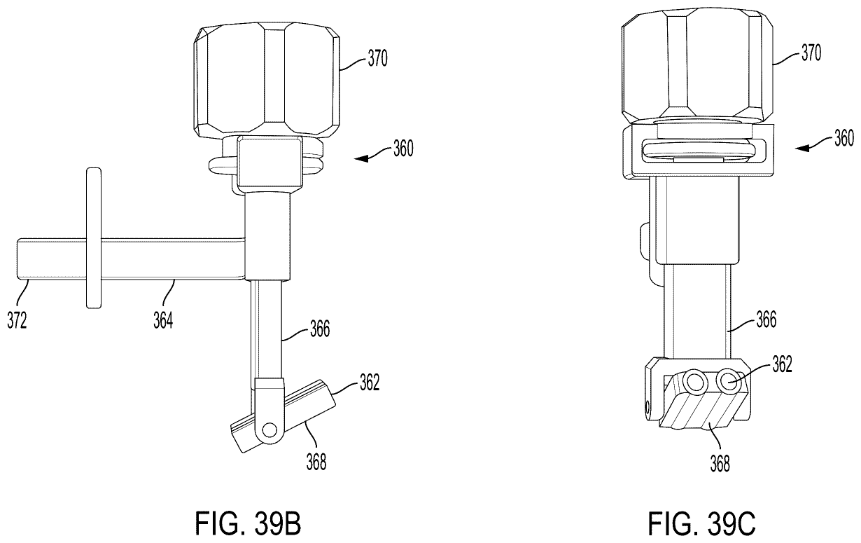

In some examples, instrument 150 is configured with a bone positioning device operable to apply a force to distal metatarsal bone portion 252 to controllably reposition the bone portion in the frontal plane (in addition to or in lieu of configuring the bone positioning device to apply a force to controllably position distal metatarsal bone portion 252 relative to proximal metatarsal bone portion 250 in the transverse plane). FIGS. 39A-39C (collectively “FIG. 39”) are views of an example bone positioning device 360 that can be used with instrument 150 to control repositioning of a bone portion (e.g., distal metatarsal bone portion 252) in the frontal plane. FIG. 39A is a perspective view of instrument 150 showing bone positioning device 360 operatively engaged with body 152 of the instrument. FIGS. 39B and 39C are side and front views, respectively, of bone positioning device 360 shown detached from instrument 150.

As shown in the example of FIG. 39, bone positioning device 360 can include at least one wire receiving opening 362 which, in the illustrated example, is shown as a pair of wire receiving openings. A wire (e.g., K-wire) inserted into distal metatarsal bone portion 252 can be received in wire receiving opening 362 to operatively connect the bone to bone positioning device 360. The body 368 defining wire receiving opening 362 can be laterally offset from the main body 152 of instrument 150. For example, bone positioning device 360 may include a dorsally extending support arm 364 and a laterally extending support arm 366 that position the body defining wire receiving opening 362 dorsally and laterally offset from the main body 152 (e.g., when instrument 150 is engaged on the medial side of the bone portions).

To control the position of the wire received through wire receiving opening 362 and, correspondingly, distal metatarsal bone portion 252 engaged therewith, bone positioning device 360 may include a force translation mechanism. In the illustrated example, body 368 defining wire receiving opening 362 can be connected through support arm 366 via a mechanical linkage (e.g., threaded rod, rack and pinion, ratchet) the controls positioning of the body. A clinician can engage the linkage, for example via an actuator or knob 370, to control the rotational position of body 368. Additionally or alternatively, the clinician can apply a hand force to control the positioning of body 386. In either case, this can control the angular alignment of wire receiving opening 362, one or more wires inserted therethrough, and the resultant position of distal metatarsal bone portion 252 in the frontal plane.

Bone positioning device 360 can be operatively connected to body 152 of instrument 150 in a variety of different ways. For example, bone positioning device 360 and body 152 may have complementary male and female connection features that allow the two components to be joined together. In the illustrated example, bone positioning device 360 is illustrated as including a male connector 372 that is inserted into a complementary female receiving opening of body 152 and can be frictionally retained therein. Another connection configurations can be used without departing from the scope of the disclosure.

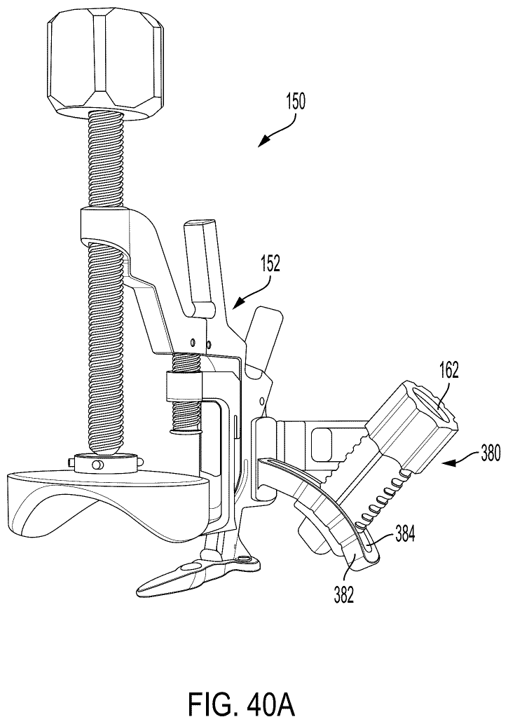

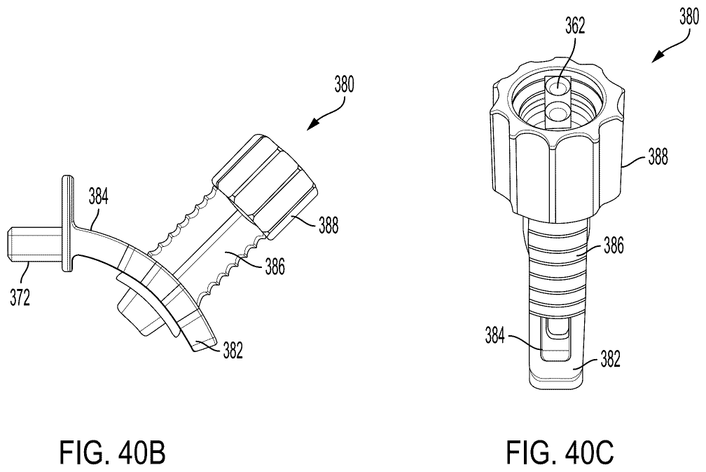

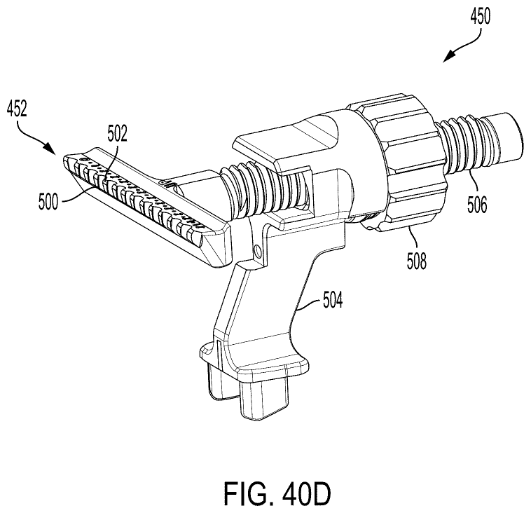

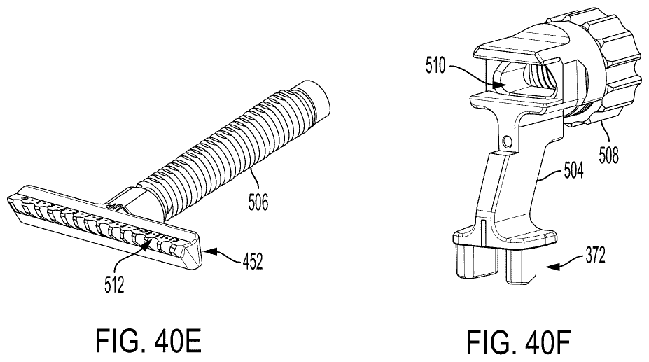

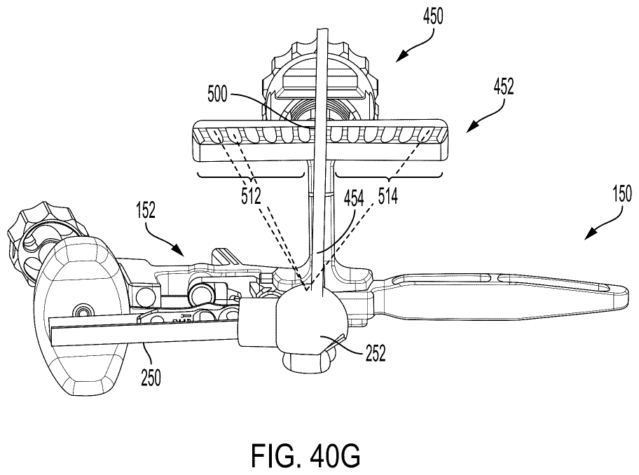

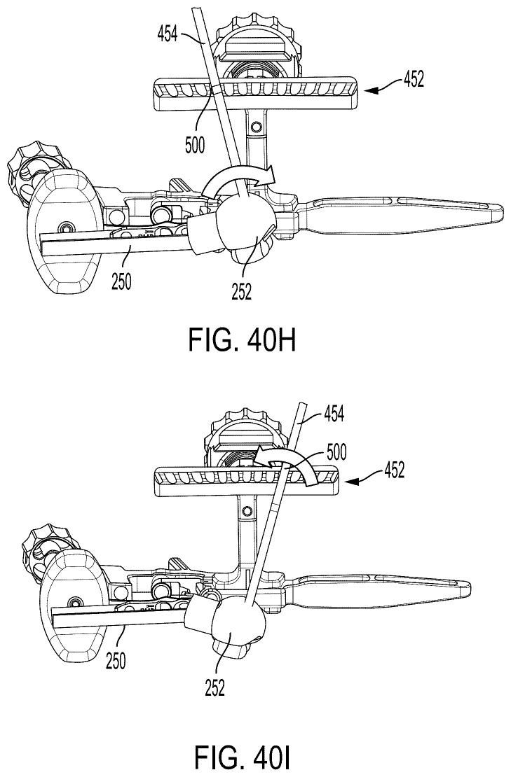

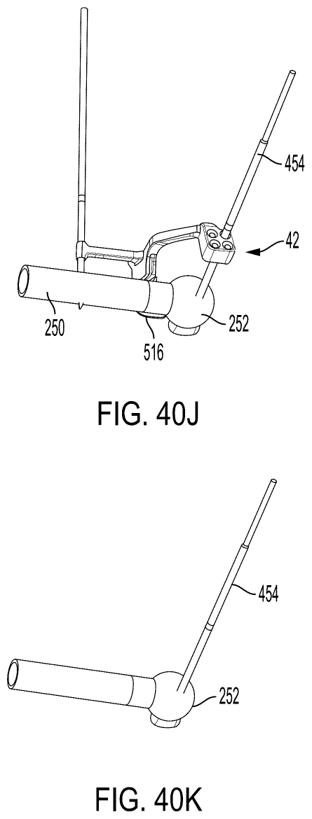

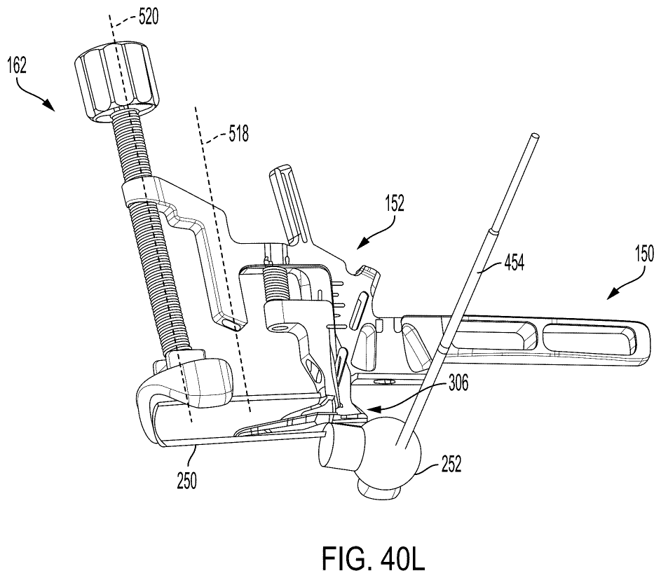

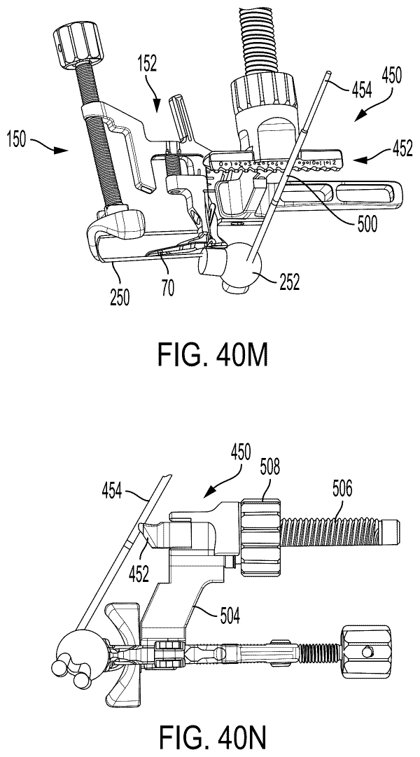

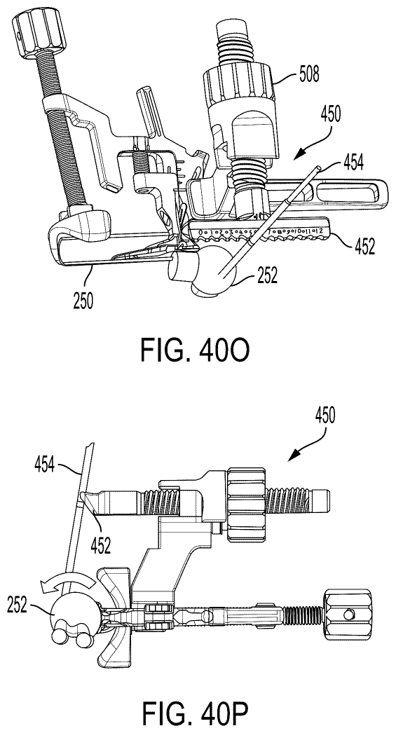

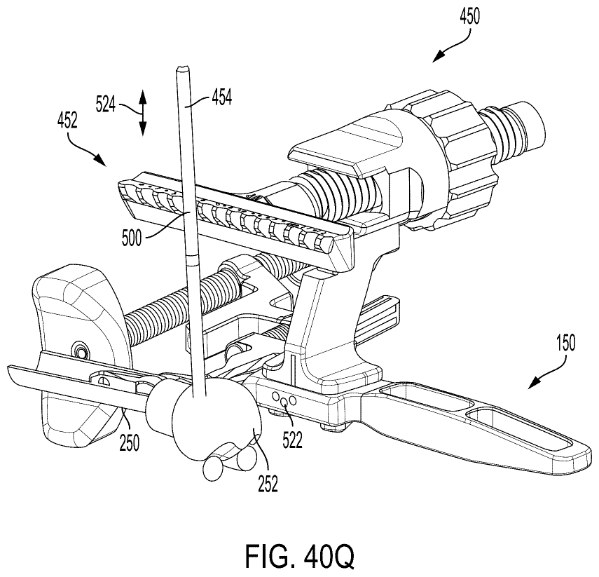

FIGS. 40A-40C (collectively “FIG. 40”) are views of an example bone positioning device 380 that can be used with instrument 150 to control repositioning of a bone portion (e.g., distal metatarsal bone portion 252) in the frontal plane. FIG. 40A is a perspective view of instrument 150 showing bone positioning device 380 operatively engaged with body 152 of the instrument. FIGS. 40B and 40C are side and front views, respectively, of bone positioning device 380 shown detached from instrument 150.

Bone positioning device 380 is similar to bone positioning device 360 in that the bone positioning device 380 is illustrated as including at least one wire receiving opening 362. A wire (e.g., K-wire) inserted into distal metatarsal bone portion 252 can be received in wire receiving opening 362 to operatively connect the bone to bone positioning device 380. Bone positioning device 380 can also be connected to body 152 any of the ways described above with respect to bone positioning device 360, including via a male connector 372 that is inserted into a complementary female receiving opening of body 152.

In the example of FIG. 40, bone positioning device 380 is illustrated as including a curved support arm 382 that can wrap dorsally and laterally away from the main body 152 (e.g., when instrument 150 is engaged on the medial side of the bone portions). Curved support arm 382 can form a track 384 that a body 386 defining wire receiving opening 362 can travel along. A locking mechanism 388 can be manipulated by user to lock a position of body 386 relative to track 384. In use, a clinician can engage locking mechanism 388 to unlock body 386 and then manipulate the position of the body along the arcuate pathway defined by support arm 382. Movement of body 386 can control the angular alignment of wire receiving opening 362, one or more wires inserted therethrough, and the resultant position of distal metatarsal bone portion 252 in the frontal plane. Once positioned at a suitable position, the clinician can engage locking mechanism 388 to secure body 386 and distal metatarsal bone portion 252 in a desired frontal plane position.

A variety of additional or different bone positioning device configurations can be used with instrument 150 to apply a force to control a position of a distal metatarsal bone portion 252 in the frontal plane. FIG. 40D is a perspective view of another example configuration of a bone positioning device 450 operable to apply a force to distal metatarsal bone portion 252 to controllably reposition the bone portion in the frontal plane. Bone positioning device 450 can engage a wire (e.g., K-wire) inserted into distal metatarsal bone portion 252 to operatively engage the bone positioning device with the bone. Bone positioning device 450 can also be permanently or detachably connected to body 152 of instrument 150 in any of the ways described above with respect to bone positioning device 360, including via a male connector 372 that is inserted into a complementary female receiving opening of body 152.

Bone positioning device 450 in the example of FIG. 40D can include a wire receiving body 452 configured to receive a wire inserted into distal metatarsal bone portion 252. Wire receiving body 452 can define one or more grooves 500 into which the wire can be received (e.g., with the side wall defining the groove at least partially extending about the wire received in the groove). For example, wire receiving body 452 may include a plurality of grooves 500 arrayed side-by-side with respect to each other (e.g., with different grooves being spaced from each other in a proximal to distal direction, when instrument 150 is in use). Each of the plurality of grooves may define a different location that can receive the wire inserted into distal metatarsal bone portion 252. Configuring wire receiving body 452 with multiple different grooves or wire receiving locations offset from each other can be useful to provide flexibility depending on where the wire inserted into distal metatarsal bone portion 252 is positioned relative to the wire receiving body.

In the illustrated example of FIG. 40D, wire receiving body 252 is shown in the form of a rake having multiple adjacent wire receiving grooves 500. For example, wire receiving body 252 may define a plurality of teeth 502 arrayed in a row. The plurality of grooves 500 may be defined by the plurality of teeth 502, with each groove being a recess relative to the projecting surfaces defined by adjacent teeth 502. Each of the grooves may be sized and shaped relative to the wire inserted into distal metatarsal bone portion 252. For example, each of the grooves 500 may define a circular shape (e.g., semi-circular wall portion) in may be sized as large or larger than the diameter of the wire inserted into distal metatarsal bone portion 252. Grooves having other sizes and shapes may also be used in the effective to receive the wire inserted into distal metatarsal bone portion 252. The specific number of wire receiving grooves 500 defined by wire receiving body 452 can vary, e.g., from 1 to 50, such as from 2 to 40, or 5 to 25.

Wire receiving body 452 can be offset from the main body 152 of instrument 150. For example, bone positioning device 450 may include an arm 504 that is configured to engage with and/or extend from instrument 150. Wire receiving body 452 can be connected to and/or carried by arm 504. For example, wire receiving body 452 can be operably connected to a shaft 506 which, in turn, is operably connected to arm 504. Wire receiving body 252 can translate relative to arm 504 and/or main body 152 of instrument 150 via shaft 506. In some configurations, wire receiving body 452 is movable along shaft 506. In other configurations, where receiving body 452 is movable with shaft 506. For example, in the illustrated configuration, wire receiving body 452 is positioned on an end of shaft 506 and moves as the shaft is translated relative to arm 504. Shaft 506 may be defined by a threaded rod, rack and pinion, ratchet, and/or other mechanical linkage that allows the position of wire receiving body 452 to be moved. In some examples, bone positioning device 450 includes an actuator 508 (e.g., rotatable knob, screw drive) that a clinician can engage to control the position of wire receiving body 452 relative to instrument 150 and/or arm 504 of the bone positioning device.

FIGS. 40E and 40F are perspective views of the example components of bone positioning device 450 shown disassembled from each other. In particular, FIG. 40E illustrates an example configuration of wire receiving body 452 and shaft 506, while FIG. 40F illustrates an example configuration of arm 504 and actuator 508. In the example of FIGS. 40E and 40F shaft 506 is illustrated as defining a threaded portion that is configured to be inserted into a corresponding receiving opening 510 of the base or arm portion 504 of the bone positioning device. In some examples, shaft 506 and receiving opening 510 define an asymmetric cross-sectional shape (e.g., a generally D-shaped body and opening) to prevent misalignment of the shaft in wire receiving body carrying thereby relative to arm 504. Receiving opening 510 can define a channel for receiving shaft 506 that can constrain movement of the shaft in a linear direction (e.g., medially to laterally) while reducing or eliminating upward or downward pitching (e.g., dorsal or plantar) and/or side to side pivoting (e.g., distal or proximal).

Each groove 500 of receiving body 452 may define a longitudinal length extending parallel to the length of the wire inserted into distal metatarsal bone portion 252 (when the wire is received in the groove). For example, each groove 500 of wire receiving body 452 may define a longitudinal length extending in a dorsal to plantar direction, when bone positioning device 450 is engaged with instrument 150 and in use. The longitudinal length of each groove 500 of wire receiving body 452 may be parallel to each other of the plurality of grooves of the wire receiving body. Alternatively, the longitudinal length of one or more grooves 500 of wire receiving body 452 may be angled relative to the longitudinal length of one or more other of the plurality of grooves 500 of the wire receiving body. Angling the longitudinal length of grooves 500 relative to each other may be useful to provide flexibility for controlling and adjusting the sagittal plane positioning of distal metatarsal bone portion 252.

FIG. 40G illustrates an example configuration of bone positioning device 450 attached to and extending from instrument 150 with the instrument engaged with both proximal metatarsal bone portion 250 and distal metatarsal bone portion 252. As illustrated, a wire 454 is inserted into distal metatarsal bone portion 252 and received by one of the plurality of grooves 500 of wire receiving body 452. Bone positioning device 450 in this example is configured to control an angle of distal metatarsal bone portion 252 in the sagittal plane relative to proximal metatarsal bone portion 250 by receiving wire 454 in different ones of the plurality of grooves 500. For example, the longitudinal length of plurality of grooves 500 may extend at different angles relative to each other. In some configurations, one of the plurality of grooves 500 (e.g., a groove at or around the middle of the array of different grooves) may extend orthogonally (at 90°) relative to main body 152 of instrument 150 and/or parallel to the dorsal-to-planar direction of the metatarsal bone portions. Grooves 500 offset from the parallel groove may be angled in the dorsal-to-planar direction, with the extent of angulation increasing moving away from the middle of the array of grooves. Wire receiving body 452 may include a first plurality of grooves 512 angled in a first sagittal plane direction and a second plurality of grooves 514 angled in a second sagittal plane direction opposite the first sagittal plane direction. The two sets of grooves may be positioned on opposite sides of the center of the array of grooves. In some configurations, the longitudinal lengths of the plurality of grooves 500 are angled to define wire receiving axes that converge at an apex point. In other configurations, the longitudinal lengths of the plurality of grooves 500 are angled to define wire receiving axes that do not converge.

FIGS. 40H and 40I illustrate bone positioning device 450 from FIG. 40G showing wire 454 positioned in different example grooves of wire receiving body 452 to adjust the sagittal plane angle of distal metatarsal bone portion 252. FIG. 40H illustrates wire 454 received in a groove 500 that plantar biases distal metatarsal bone portion 252. Inserting wire 454 into a plantar biases groove can cause the proximal end of distal metatarsal bone portion 252 to angle plantarly (downwardly) and/or the distal end of the distal metatarsal bone portion to angle dorsally (upwardly). The clinician can move the position of wire 454 to a different one of the plurality of grooves to adjust the sagittal plane angulation of distal metatarsal bone portion 252 (e.g., optionally moving the wire to a position that causes the distal metatarsal bone portion to be substantially coplanar with the proximal metatarsal bone portion 250). FIG. 40I illustrates wire 454 received in a groove 500 that dorsal biases distal metatarsal bone portion 252. Inserting wire 454 into a dorsal biasing groove can cause the proximal end of distal metatarsal bone portion 252 to angle dorsally (upwardly) and/or the distal end of the distal metatarsal bone portion to angle plantarly (downwardly). Again, the clinician can move the position of wire 454 to a different one of the plurality of grooves to adjust the sagittal plane angulation of distal metatarsal bone portion 252.

FIGS. 40J-40Q illustrate example surgical technique steps that can implemented to treat a bunion deformity using example instruments according to the disclosure. As shown in FIG. 40J, the example technique can involve attaching a bone preparation guide 42 to a metatarsal with one or more wires 454 inserted distally of a target cut location 516 an optionally with one or more other wires inserted proximally of the target cut location. At least one wire 454 inserted distally of the target cut location 516 may extend generally perpendicularly to the dorsal surface of the metatarsal (e.g., such that the wire extends dorsally to plantarly). In some examples, bone preparation guide 42 is positioned to locate a wire receiving opening of the guide at a location over a dorsal surface of a distal portion of the metatarsal and wire 454 is thereafter advance through the wire receiving opening. In other examples, wire 454 is inserted through a dorsal surface of the distal portion of the metatarsal and bone preparation guide 42 is thereafter placed over the wire. In either case, the clinician can then guide a cutting instrument using the bone preparation guide 42 to divide the metatarsal into proximal metatarsal bone portion 250 and distal metatarsal bone portion 252.

As shown in FIG. 40K, after cutting the metatarsal into proximal metatarsal bone portion 250 and distal metatarsal bone portion 252, bone preparation guide 42 and any extra fixation wires can be removed leaving at least one wire 454 inserted into distal metatarsal bone portion 252. As will be described, this wire 454 may be engaged with a frontal plane bone positioning device, such as bone positioning device 450. Because wire 454 may receive and transmit a comparatively high force to control positioning of distal metatarsal bone portion 252, the wire selected to be used as wire 454 may have a sufficiently large diameter for the application. In various examples, wire 454 may have a diameter of at least 1.0 mm, such as at least 1.2 mm, at least 1.6 mm, at least 2.0 mm, at least 2.2 mm, or at least 2.4 mm.

FIG. 40L illustrates an example configuration of instrument 150 having a body 152 releasably connected to an intramedullary implant 70, a bone positioning device 162 operatively connected to the body and configured to apply a transverse plane pushing force to proximal metatarsal bone portion 250, and an intramedullary insertion body 306 operatively connected to the body and configured to apply a transverse plane pulling force to the proximal metatarsal bone portion 250. As shown in the illustrated example, the stem portion of intramedullary implant 70 is inserted into proximal metatarsal bone portion 250, and the plate portion of the intramedullary implant is positioned pressing against the surface (e.g., a medial side surface) of distal metatarsal bone portion 252. Bone positioning device 162 and/or intramedullary insertion body 306 can be engaged to push distal metatarsal bone portion 252 and/or pull proximal metatarsal bone portion 250 in the transverse plane relative to each other (e.g., to move distal metatarsal bone portion 252 laterally relative to proximal metatarsal bone portion 250 and/or to maximize the amount of offset between the cut end faces of the two metatarsal bone portions), as discussed above.

Before, after, and/or while moving distal metatarsal bone portion 252 relative to proximal metatarsal bone portion 250, distal metatarsal bone portion 252 may be moved in the frontal plane and/or sagittal plane using a frontal plane and/or sagittal plane bone positioner, such as bone positioner 450. In some implantations, one or fixation wires are inserted through instrument 150 and into proximal metatarsal bone portion 250 (before, after, and/or during bone repositioning in the transverse, frontal, and/or sagittal planes). For example, body 152 of instrument 150 may define an opening (e.g., for guiding a screw through the stem portion of intramedullary implant 70) that can be used to insert a fixation wire 518 for pinning the instrument to proximal metatarsal bone portion 250. Additionally or alternatively, bone positioning device 162 can include an opening 198 as described above for receiving a fixation wire 520 for pinning the instrument to proximal metatarsal bone portion 250. In either case, pinning instrument 150 to proximal metatarsal bone portion 250 can help stabilize the instrument relative to proximal metatarsal bone portion 250 and/or distal metatarsal bone portion 252.

As shown in FIGS. 40M and 40N, bone positioning device 450 can be connected to the body 152 of instrument 150 (in configurations in which the bone positioning device 450 is attachable to and detachable from body 152). Wire receiving body 452 of bone positioning device 450 can be advanced until wire 454 is received in one of the plurality of grooves 500 of the wire receiving body. For example, the clinician can engage actuator 508 to translate shaft 506 relative to first arm viable for, causing wire receiving body 452 two advance until the way receiving body contexts wire 454.

In the illustrated arrangement, body 152 of instrument 150 is shown positioned on a medial side of the proximal metatarsal bone portion 250 and on a medial side of distal metatarsal bone portion 252 with the stem portion of intramedullary implant 70 inserted into proximal metatarsal bone portion 250 and the plate portion of intramedullary implant 70 inserted against the distal metatarsal bone portion 252. When so positioned, arm 504 of bone positioning device 450 can be positioned to extend in a dorsal direction from body 152 and shaft 506 of the bone positioning device can be positioned to extend in a lateral direction from the arm. As a result, bone positioning device 450 can apply a laterally-directed force to wire 454 to move the distal metatarsal bone portion 252 in at least a frontal plane.

FIGS. 40O and 40P are dorsal and frontal plane views, respectively, illustrating example repositioning of distal metatarsal bone portion 252 in the frontal plane using bone positioning device 450. As shown, wire receiving body 452 can be advanced (e.g., laterally) against wire 454, inducing a moment at the distal metatarsal bone portion 252 through the wire that causes the distal metatarsal bone portion 252 to rotate in the frontal plane. The clinician can advance wire receiving body for 52 by controlling actuator 508 until distal metatarsal bone portion 252 is at a desired frontal plane rotation position. In some instances, this involves repositioning the sesamoid bones plantarly and parallel to ground under distal metatarsal bone portion 252. In some examples, the clinician may over rotate distal metatarsal bone portion 252 such that the sesamoid bones under the distal metatarsal bone portion are shifted from directly plantar to being angled at least partially medially. Over rotation may be beneficial if spring back or derotation is anticipated later in the procedure. In some examples, the clinician rotates distal metatarsal bone portion 252 in the frontal plane to substantially realign the rotational position of the distal metatarsal bone portion and/or sesamoid bones back to their normal anatomical frontal plane rotation position, e.g., such as the position as observed in normal patient population not experiencing a bunion deformity.

With reference to FIG. 40Q, before, during, and/or after moving distal metatarsal bone portion 252 in the frontal plane, the clinician can optionally move the distal metatarsal bone portion 252 in the sagittal plane to correct the sagittal plane alignment of the metatarsal bone portion. For example, the clinician can move wire 454 from one of the plurality of grooves 500 to a different one of the plurality of grooves 500. Where the grooves are angled relative to each other, shifting wire 454 proximally or distally along the array of grooves from one group to a different groove can cause the angular orientation of pin 454 to change, and correspondingly change in the sagittal plane angulation of distal metatarsal bone portion 252 (as discussed above with respect to FIGS. 40G-40I). This can change the angular orientation of the cut end face of distal metatarsal bone portion 252 relative to the cut end face of proximal metatarsal bone portion 250 in the sagittal plane. Typically, the clinician can adjust the angular orientation of distal metatarsal bone portion 252 so the distal metatarsal bone portion is substantially in line with (e.g., coaxial with) proximal metatarsal bone portion 250 and isn't pitched dorsally or plantarly. For example, during the procedure, tissue surrounding proximal metatarsal bone portion 250 and/or distal metatarsal bone portion 252 can introduce misalignments to distal metatarsal bone portion 252 as other portions of the procedure are performed (e.g., resulting in distal metatarsal bone portion 252 angling proximally or distally) which can be corrected during sagittal plane adjustment.

Additionally or alternatively, the clinician can shift the entirety of distal metatarsal bone portion 252 dorsally or plantarly in the sagittal plane (in addition to or in lieu of change in the angular orientation of the cut end face of the metatarsal bone portion). For example, the clinician can apply a force to wire 454 in the direction indicated by arrow 524, advancing the wire plantarly to move distal metatarsal bone portion 252 plantarly or advancing the wire dorsally to move distal metatarsal bone portion 252 dorsally. The clinician can move wire 454 dorsally or plantarly while the wire is retained in one of the plurality of grooves 500. The clinician can adjust the orientation of distal metatarsal bone portion 252 so the distal metatarsal bone portion is substantially in line with (e.g., coaxial with) proximal metatarsal bone portion 250. If desired, a securing mechanism can be used to lock wire 454 at a particular location and/or orientation relative to wire receiving body 452 to prevent inadvertent movement of the wire (and correspondingly distal metatarsal bone portion 252) after the distal metatarsal bone portion 252 has been moved to a desired frontal plane and/or sagittal plane position. For example, bone positioning device 450 may include a clamp, snap, or other mechanical fixation figure that locks the position of the wire relative to the device. Additionally or alternatively, an external locking mechanism may be applied to wire 454 (e.g., a Kocher clamp) to help prevent plantar sliding of the wire.

To temporarily fixate the position of distal metatarsal bone portion 252 relative to proximal metatarsal bone portion 250 after realignment, one or more temporary fixation wires can be inserted into the distal metatarsal bone portion through one or more corresponding fixation apertures 522 extending through the instrument 150. For example, instrument 150 may include multiple openings 522 to allow one or fixation wires to be inserted therethrough and into distal metatarsal bone portion 252. The one or more wires can be inserted through the medial side of instrument 150 laterally such that the one or more wires penetrate the medial side of distal metatarsal bone portion 252. After distal metatarsal bone portion 252 is temporarily fixated using the wires, the moved position of the distal metatarsal bone portion can be permanently fixated by inserting screws into intramedullary implant 70 in the temporary fixation wires removed.

Before temporarily and/or permanently fixating the moved position of distal metatarsal bone portion 252 relative to proximal metatarsal bone portion 250, the clinician may manipulate the distal metatarsal bone portion 252 and/or proximal phalanx to control the distal metatarsal articular angle (DMAA). The DMAA is the angle formed between the longitudinal axis and the distal articular surface of the first metatarsal and is used to assess the metatarsophalangeal joint coverage or joint congruity. In some applications, the clinician holds the tip of the toe out immediately to correct the DMAA before applying fixation.

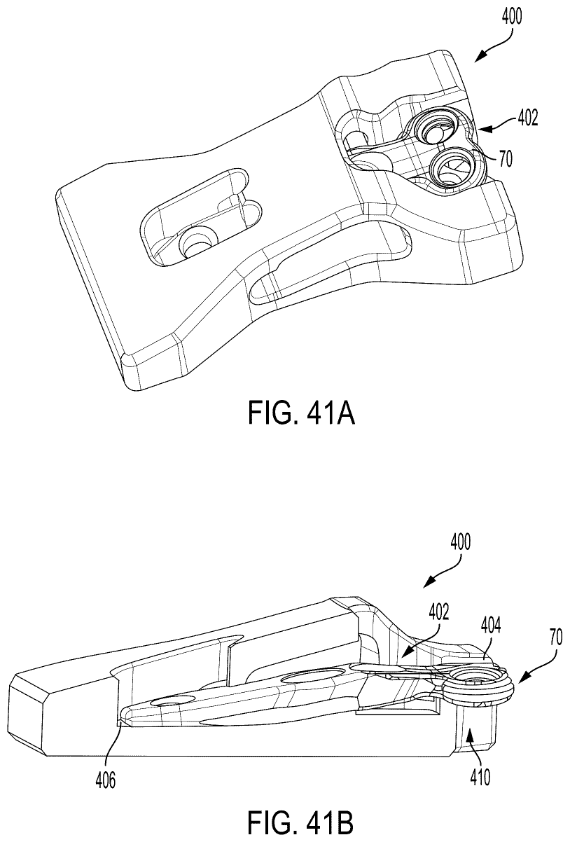

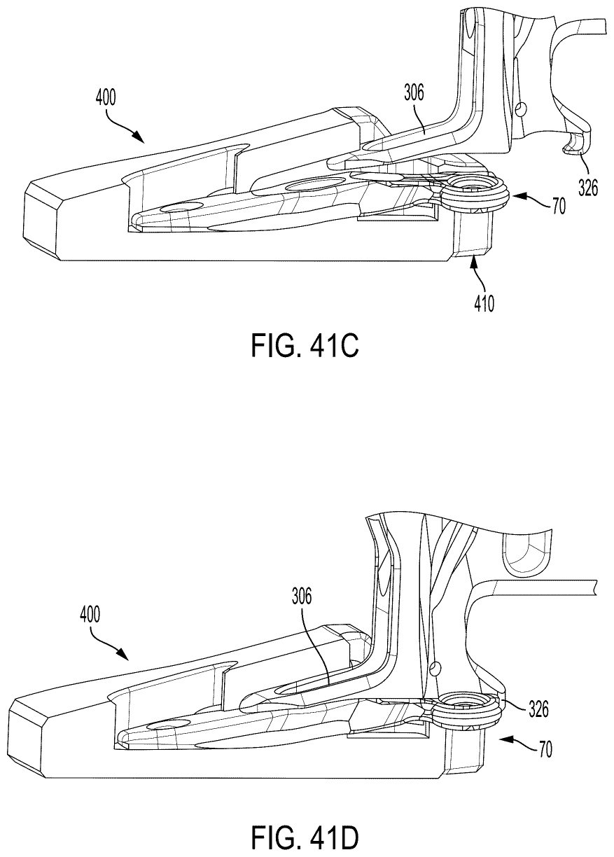

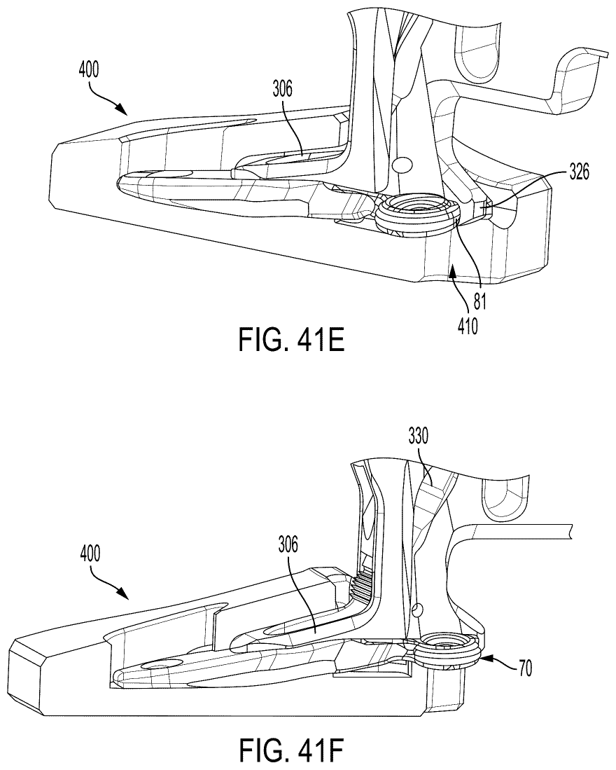

A surgical procedure according to the disclosure may utilize a variety of auxiliary tools in addition to or in lieu of instrument 150 to help facilitate efficient and accurate execution of the procedure. FIGS. 41A-41F (collectively “FIG. 41”) are different images of an example loading block 400 that can be used to load intramedullary implant 70 onto instrument 150. Loading block 400 can aid a clinician to properly position the implant relative to the instrument to ensure positioning and engagement of the implant to the instrument.

As shown in FIG. 41, loading block 400 may define a receiving cavity 402 configured to receive intramedullary implant 70 and at least a portion of instrument 150 to interconnect the implant to the instrument. Receiving cavity 402 can be sized and shaped corresponding to the size and shape of the specific intramedullary implant 70 to be loaded onto instrument 150. Receiving cavity 402 can extend from a first end 404 to a second end 406. Intramedullary implant 70 can be advanced into receiving cavity, e.g., until the leading end of the implant is adjacent to and/or contacts a wall surface bounding the second end 406 of the receiving cavity. Loading block 400 may include a cutout at first end 404 size and shape to receive the lobes of intramedullary implant 70. Loading block 400 may also defined a recess 410 configured to receive hook 326 of instrument 150. The depth of receiving cavity 402 can be effective to receive the thickness of intramedullary implant 70 as well as the thickness of intramedullary insertion body 306 of instrument 150.

In use as shown in FIGS. 41A and 41B, a clinician can insert intramedullary implant 70 into receiving cavity 402. The clinician can insert the long tail of the implant into the block until the ears of the implant are seated. As shown in FIG. 41B, the thickness of receiving cavity 402 may taper such that the bone facing surface and opposite surface of the intramedullary stem of the implant contact opposed wall surfaces inside the block. With intramedullary implant 70 seated into receiving cavity 402, the clinician can insert intramedullary insertion body 306 of instrument 150 into the block, as shown by FIGS. 41C and 41D. The clinician can advance intramedullary insertion body 306 until hook 326 engages the back end of intramedullary implant 70. Receiving cavity 402 can be sized tight enough such that inserting intramedullary insertion body 306 also aligns hook 326 with the groove 81 (FIG. 13D, 13E) of the implant, as shown in FIG. 41E. Thereafter, the clinician can advance attachment rod through intramedullary implant body 306 and an opening in the top surface of loading block 400 and threading the engage implant 70, as shown in FIG. 41F.

As discussed above, a broach instrument 60 that can be used to form a pocket in the end of a metatarsal bone portion to prepare the metatarsal bone portion to receive the stem of the intramedullary implant. FIGS. 42A and 42B (collectively “FIG. 42”) are images of an example configuration of a trial implant and broach 60 can be used to form a pocket in the end of a metatarsal bone portion to prepare the metatarsal bone portion to receive the stem of the intramedullary implant. In this example, broach instrument 60 includes a handle 62 graspable by a user to manipulate the broach instrument and an insertion body 420 configured to be inserted in the end of a metatarsal bone portion. Insertion body 420 may function as both a pocket-forming instrument and a trialing instrument. Accordingly, insertion body 420 may be implant sized and shaped to correspond to the size and shape of intramedullary implant 70 (optionally including the additional thickness of intramedullary implant body 306 from instrument 150 that overlies intramedullary implant in is inserted into the medullary canal of the bone).

For example, insertion body 420 can have a length extending from a first end 422 to a second end 424 corresponding to the length of intramedullary implant 70. Insertion body 420 can have a width that tapers from second end 424 to first end 422 corresponding to an angle of taper of intramedullary implant 70 over the corresponding length of the implant. Further, insertion body 420 can have a thickness corresponding to the thickness of intramedullary implant 70 and/or the combination of intramedullary implant body 306 and intramedullary implant 70 over a corresponding length.

In use, the clinician can manipulate handle 62 and insertion body 420 into the cut end face of a bone portion, such as proximal metatarsal bone portion 250. Insertion body 420 can form a pocket in the bone portion sized and shaped subsequently receive intramedullary stem portion 72 of intramedullary implant 70 and/or intramedullary implant body 306 of instrument 150. The clinician can visualize insertion body 420 in the bone portion, for example under fluoroscopy, to evaluate the fit and positioning of the insertion body. The clinician can decide based on the visualization of insertion body 420 functioning as a trial implant whether the selected implant is appropriately sized and positioned. Additionally or alternatively, the clinician can place insertion body 420 against an external surface of a bone portion and visualize the length and/or size of the insertion body relative to the bone portion to determine the appropriateness of the corresponding implant sizing. In some examples, the clinician may be provided with a system of different broaches 60 each having a different sized insertion body 420 corresponding to different sized intramedullary implant 70 available for the clinician to use.

Insertion body 420 can have a variety of different design features. For example, insertion body 420 may be configured with a plurality of cutting teeth 426 are arrayed across one or more surfaces of the insertion body to help cut a pocket in the bone portion into which the insertion body is inserted. Cutting teeth 426 may be angled so the teeth cut upon insertion of the insertion body 420 into the end face of the bone.

As another example, insertion body 420 may include a wire receiving opening 427 for receiving a K-wire to pin the body to an underlying bone portion. Additionally or alternatively, insertion body 420 may include a cut line indicator feature 428 that can be used to determine where to cut a metatarsal such that plate 74 lands distal of the cut line and intramedullary stem portion 72 lands proximal of the cut line.

FIGS. 43A-43D illustrate example surgical technique steps that can be used when performing a procedure with broach 60. With reference to FIG. 43A, a clinician can place insertion body 420 against an external surface of first metatarsal 210, such that the second end 426 of the insertion body is adjacent to an/or in contact with the head of the metatarsal. This can align cut line indicator feature 428 relative to the metatarsal. With reference to FIG. 43B, the clinician may advance a K-wire through wire receiving opening 427 such that the wire exits out at cut line indicator feature 428 and is inserted into metatarsal 210. Thereafter, the clinician can remove insertion body 420 and guide a cutting guide over the K-wire to cut the metatarsal into a proximal metatarsal bone portion 250 and the distal metatarsal bone portion 252, as discussed herein. With the metatarsal cut into two portions, the clinician can insert insertion body 420 into the cut end of proximal metatarsal bone portion 250 until reaching a depth stop 430 corresponding to the depth to which intramedullary implant 70 can be inserted, as seen in FIG. 43C. Thereafter, the clinician can withdraw insertion body 420 and insert intramedullary implant 70 attached to instrument 150, thereby positioning intramedullary stem portion 72 in a pocket formed by insertion body 420 having matched size and shape, as seen in FIG. 43D.

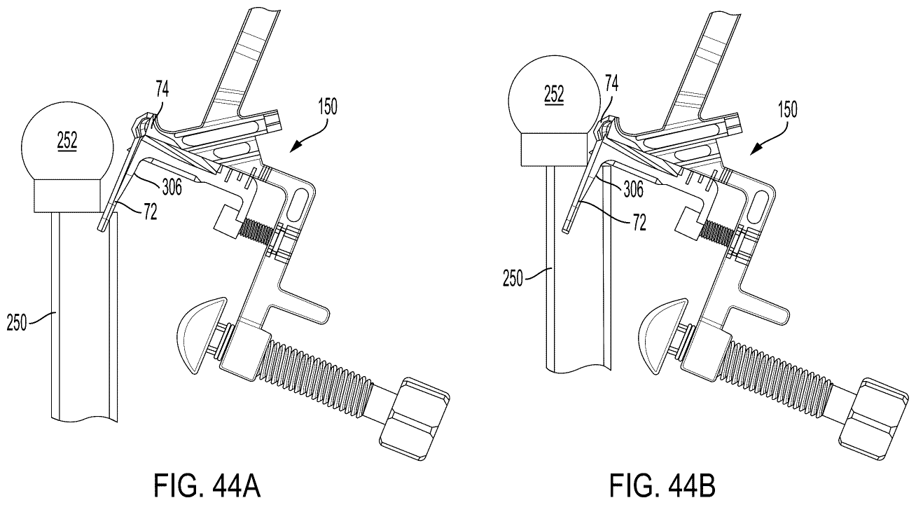

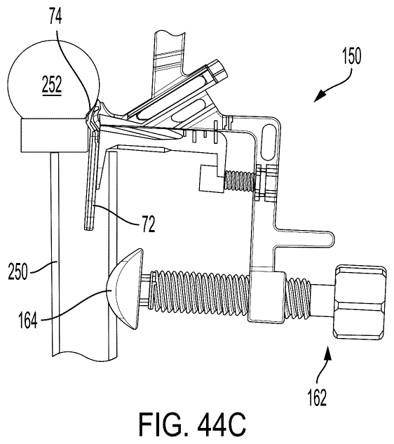

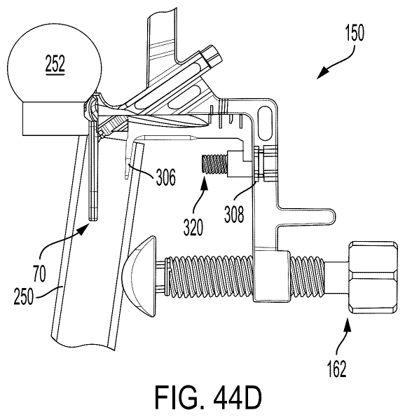

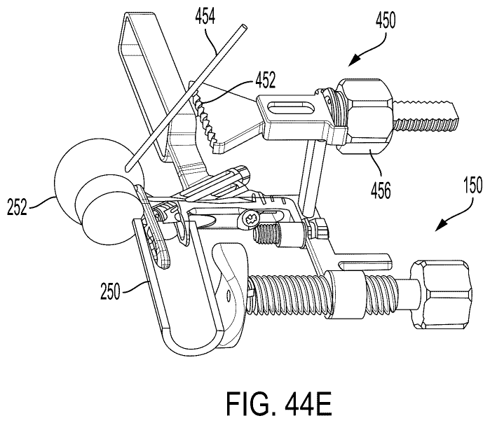

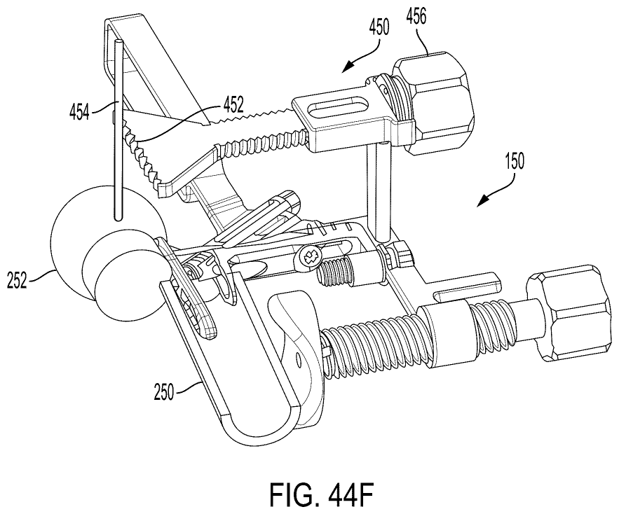

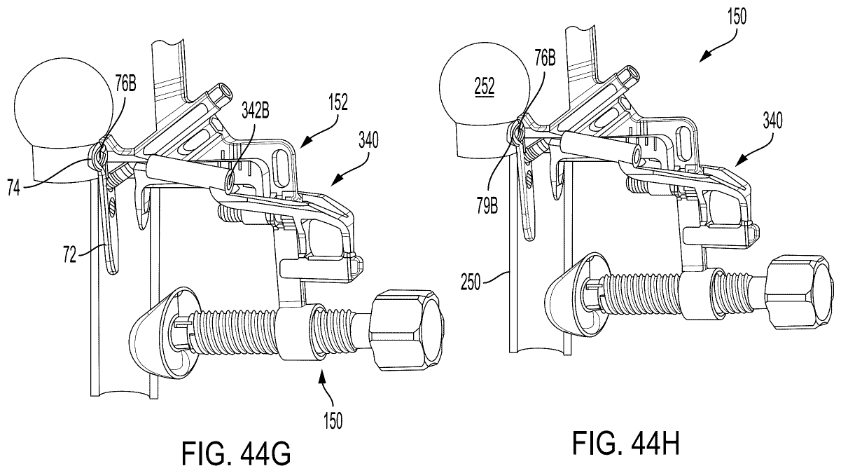

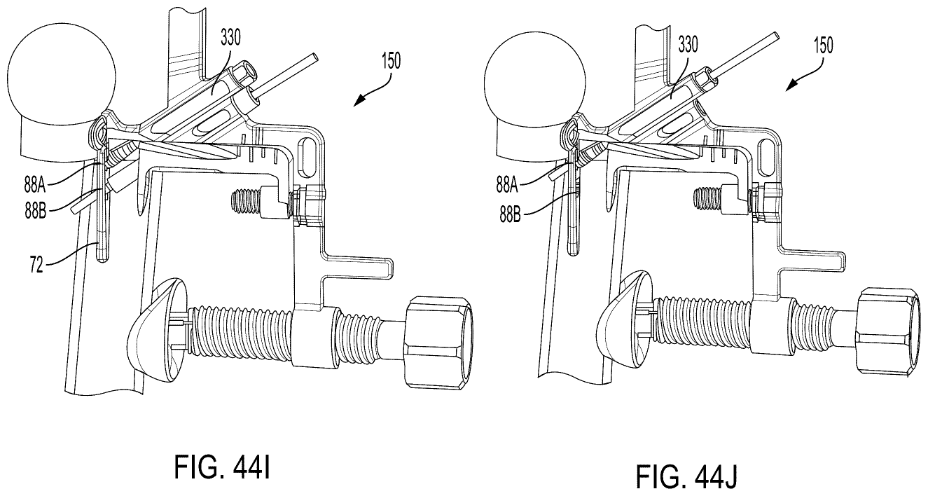

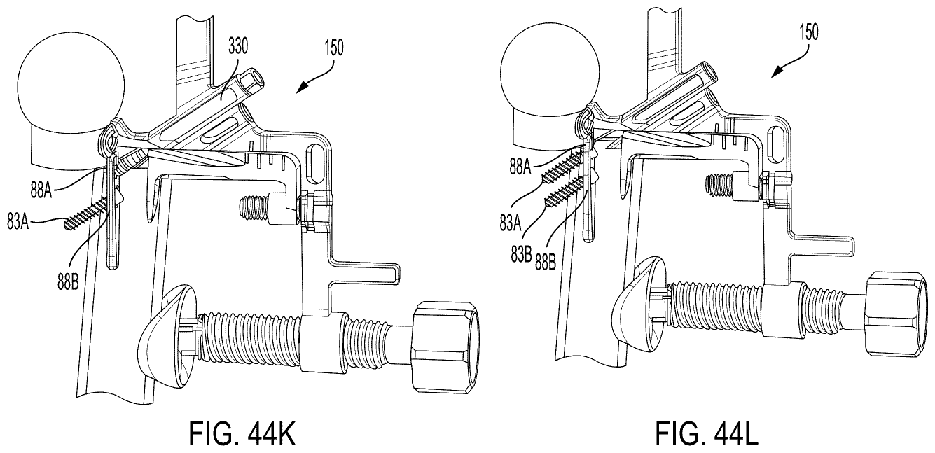

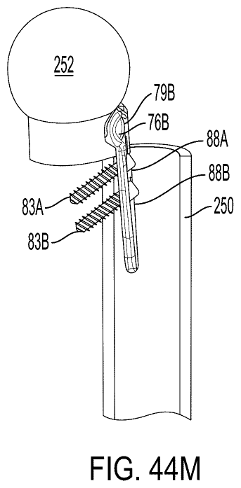

FIGS. 44A-44M illustrate example implant insertion and bone realignment procedure steps that may be performed using an example intramedullary implant 70 and instrument 150 according to the disclosure. With reference to FIGS. 44A and 44B, after cutting the metatarsal into a proximal metatarsal bone portion 250 and distal metatarsal bone portion 252, the clinician can insert intramedullary stem portion 72 of intramedullary implant 70 attached to instrument 150 through the cut end face of proximal metatarsal bone portion 250. As intramedullary stem portion 72 is inserted through the cut end face, intramedullary insertion body 306 of instrument 150 is also inserted through the cut end face of proximal metatarsal bone portion 250 and into the medullary canal. Intramedullary stem portion 72 and intramedullary insertion body 306 may initially be inserted at an angle and, as the components are inserted into the medullary canal, cause distal metatarsal bone portion 252 to be pushed laterally away from proximal metatarsal bone portion 250 in the transverse plane. Instrument 150 can then be rotated to rotate intramedullary stem portion 72 to be parallel to the longitudinal axis of proximal metatarsal bone portion 250.

With reference to FIG. 44C, the clinician can engage bone positioning device 162 of instrument 150 to apply a force the contact surface 164 two proximal metatarsal bone portion 250. This can cause a portion of intramedullary implant 70 and/or a portion of instrument 150 overlying distal metatarsal bone portion 252 to press against the distal metatarsal bone portion and push the distal metatarsal bone portion laterally in the transverse plane. The clinician can advance bone positioning device 162 until transverse plane correction of distal metatarsal a portion 252 is achieved.

With reference to FIG. 44D, before, after, and/or while actuating bone positioning device 162, the clinician can engage intramedullary insertion body translation mechanism 320. For example, the clinician can engage a driver or knob to turn a threaded shaft attached to intramedullary insertion body 306 to draw the implant insertion body toward support surface 308 of instrument 150. This can cause intramedullary insertion body 306 to move medially within the medial canal of proximal metatarsal bone portion 250. As a result, distal metatarsal bone portion 252 can be shifted further laterally within the transverse plane and/or proximal metatarsal bone portion 250 further transversely within the transverse plane relative to the other bone portion.

With reference to FIGS. 44E and 44F, before, after, and/or while moving distal metatarsal bone portion 252 relative to proximal metatarsal bone portion 250 in the transverse plane (e.g., using positioning device 162), the clinician can move the distal metatarsal bone portion in the frontal plane and/or sagittal plane (e.g., using bone positioning device 450). While the frontal plane positioner can have a variety of different configurations as discussed herein, FIGS. 44E and 44F illustrate instrument 150 having an example positioner 450 in the form of a rake 452 that can apply a force to a K-wire 454 inserted into distal metatarsal bone portion 252. A clinician can engage a driver or actuator 456 to apply a force to K-wire 454 via positioner 450, causing the projecting end of the wire to rotate (e.g., laterally) in the frontal plane.

With distal metatarsal bone portion 252 repositioned in one or more planes, the clinician can fixate the moved position of the distal metatarsal bone portion using intramedullary implant 70. For example, with reference to FIGS. 44G and 446 H, the clinician can slide detachable drill guide 340 onto body 152 of instrument 150. The clinician can then use the guide apertures of drill guide 340 to drill holes and/or insert screws through the apertures in plate 74, thereby securing the plate to distal metatarsal bone portion 252 with the screws.

With reference to FIGS. 441-44K, the clinician can prepare and fixate intramedullary stem 72. The clinician can insert a drill pin, such as a drill bit, through a guide aperture of instrument 150 to prepare a proximal-most aperture of intramedullary stem portion 72 to receive a corresponding screw. Before or after preparing the proximal-most aperture by drilling, the drill pin can be inserted down attachment rod 330 to prepare a distal aperture of intramedullary stem portion 72 to receive a corresponding screw. The clinician can advance a screw through the guide aperture of instrument 150 proximal-most aperture of intramedullary stem 72. The clinician can then withdraw attachment rod 330 and advance another a screw through the guide aperture previously occupied by attachment rod 330 into the distal aperture of intramedullary stem 72. When screw attachment is complete, the clinician can remove instrument 150, leaving intramedullary implant 70 fixated to the two bone portions as seen in FIG. 44M.

FIGS. 47A-47G illustrate another set of example procedural steps that may be performed using an example intramedullary implant 70 and instrument 150 according to the disclosure. The figures are illustrated with an example configuration of intramedullary implant 70 includes an example tail aperture 88C, as discussed above with respect to FIGS. 13F and 13G, although can be performed with intramedullary implants having other configurations. The figures illustrate example fixation steps that can be performed after distal metatarsal bone portion 252 has been repositioned in one or more planes.

With reference to FIG. 47A, instrument 150 is illustrated engaged with proximal metatarsal bone portion 250 and distal metatarsal bone portion 252. With correction of distal metatarsal bone portion 252 complete, one or screws 79A, 79B are illustrated as having been inserted through the plate portion of intramedullary implant 70 into the distal metatarsal bone portion. In addition, one or more screws 83A are illustrated as having then inserted through the stem portion of intramedullary implant 70. A fixation wire 518 (FIG. 40L), tack 91 (FIG. 13G), and/or drill bit can be advanced through an opening in the body of instrument 150 to create a hole through fixation aperture 88C detailed portion of the intramedullary stem. The opening form by the instrument can be unicortical, for example extending through a medial cortex of proximal metatarsal portion 250, or bicortical, for example extending through both a medial cortex and a lateral cortex of the proximal metatarsal portion. Once the hole is created, the instrument can be removed from aperture 88C and screw 83C inserted, as illustrated in FIG. 47B.

In some examples, tack 91 is removed and screw 83C is guided into the opening formed by the tack (e.g., through a guiding opening defined by the body of instrument 150). In some examples, a cannulated drill is used in addition to or in lieu of tack 91 to create the opening through fixation aperture 88C and wire is left extending through the fixation aperture after removal of the drill bit. A cannulated screw 83C can then be advanced alone the wire to guide placement of the screw in and through fixation aperture 88C. Additionally or alternatively, a sleeve may be introduced through the guiding opening defined by the body of instrument 150 and used to guide a wire through fixation aperture 88C (followed by guiding a cannulated screw 83 over the wire) and/or to guide screw 83 through the sleeve.

Screw 83C can have a variety of different configurations, and can be a locking screw or nonlocking screw, and can extend unicortically or bicortical through one or both cortical wall of proximal metatarsal portion 250. FIG. 47C is an illustration of an example tail screw 83C being inserted through an example tail screw aperture 88C of intramedullary implant 70. FIG. 47D illustrates the resulting example construct after removal of instrument 150. As shown in these examples, tail screw 83C may be configured as a bicortical screw configured to threadably engage both a lateral cortical wall 650 and a medial cortical wall 652 of proximal metatarsal bone portion 250. Tail screw 83C can include a first region of threading 654 configured to threadably engage lateral cortical wall 650 and a second region of threading 656 configured to threadably engage medial cortical wall 652. The two regions of threading may be different regions of a continuous threading extending along the length of screw 83C or may be separated by one or more regions devoid of threading. For example, screw 83C may include a region devoid of threading that is configured to be positioned aligned with (e.g., in plane with) the region of intramedullary implant 70 defining fixation aperture 88C. In these examples, fixation aperture 88C may be an unthreaded opening.

FIG. 47E illustrates another example configuration of tail screw 88C in which the screw is configured to threadably engage one or both cortical walls while also locking into threading surrounding fixation aperture 88C. In particular, FIG. 47E illustrates tail screw 88C including a first region of threading 654 configured to threadably engage lateral cortical wall 650, a second region of threading 656 configured to threadably engage medial cortical wall 652, and a third region of threading 658 configured to threadably engage corresponding threading of fixation aperture 88C. In this example, second region of threading 656 and third region of threading 658 are continuous, although in other examples, one or more regions devoid of threading may separate the different regions of threading. In some examples, third region of threading 658 is configured to threadably engage corresponding threading of fixation aperture 88C without locking into the fixation aperture threading. In other examples, third region of threading 658 is configured to threadably engage corresponding threading of fixation aperture 88C and lock into the fixation aperture threading. For example, the third region of threading 658 may taper to provide a tighter constraint of the screw relative to the fixation aperture 88C and interlock the screw to the implant. FIG. 47F is an example illustration showing screw 88C from FIG. 47E threadably engaged with lateral cortical wall 650, threadably engaged with medial cortical wall 652, and threadably engage corresponding threading of fixation aperture 88C.

FIG. 47G illustrates another example configuration of tail screw 88C in which the screw is configured as a unicortical screw configured to extend through one but not both cortical walls of proximal metatarsal bone portion 250. In particular, in the illustrated example, tail screw 88C is illustrated as having a head 660 that is configured to be advanced into the medullary canal of proximal metatarsal bone portion 250 until the head is seated on and/or in fixation aperture 88C. Head 660 and/or fixation aperture 88C as a locking screw arrangement in which the head threadably engages corresponding threading of fixation aperture 88C or as a non-locking arrangement, such as a compression screw arrangement (e.g., in which head 660 and/or fixation aperture 88C are devoid of interlocking threading). In this example, tail screw 88C includes a threaded shaft configured to extend through the lateral cortical wall 650 of proximal metatarsal bone portion 250. Configuring screw 88C to have a head positioned beyond the medial cortical wall 652 may be useful to allow for more aggressive removal of the residual medial aspect of proximal metatarsal bone portion 250, as indicated by the medial aspect removal line 670, without screw interference.

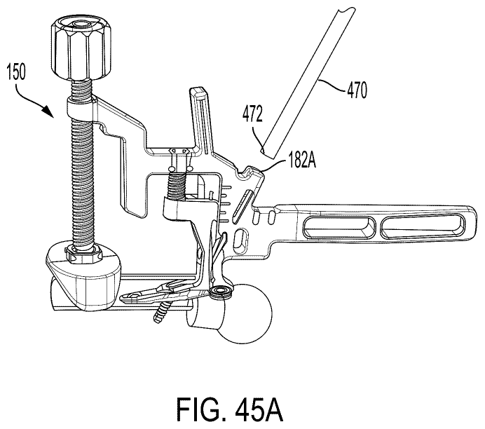

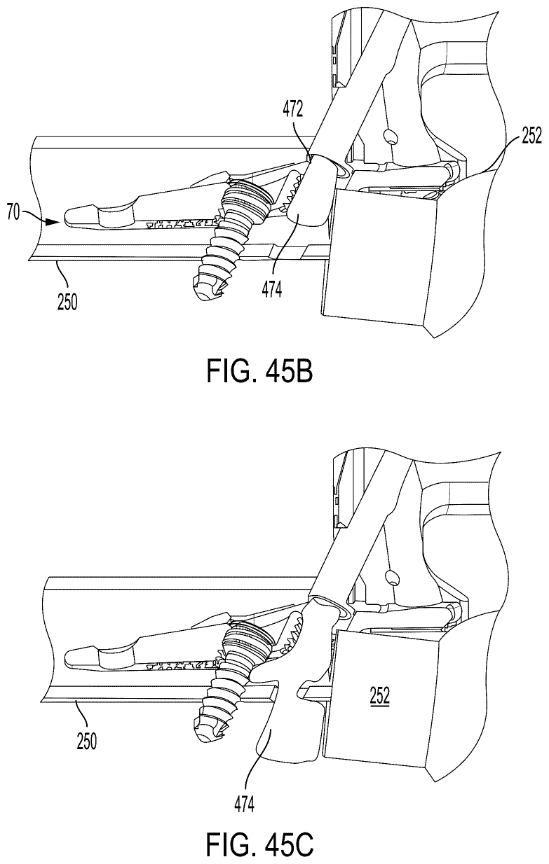

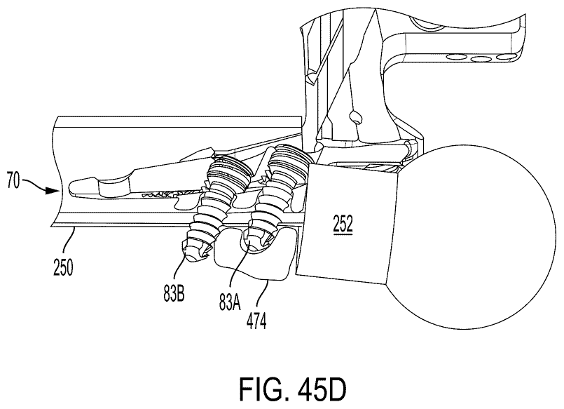

In some examples, a clinician may introduce a biologic into the space between proximal metatarsal bone portion 250 and distal metatarsal bone portion 252 before, during, and/or after complete attachment of intramedullary implant 70 to promote healing and bone in growth between the two bone portions. FIGS. 45A-45D illustrate example procedural steps that can be used to introduce a biologic to a joint space using instrument 150. As shown, a tube 470 can be advanced down one or guide apertures defined through instrument 150 to position the outlet 472 of tube at the osteotomy location. One or more biologic materials 474 can then be delivered through the tube to the osteotomy site. In some examples, the tube is withdrawn and a screw subsequently inserted through the aperture previously receiving the tube and fixated to the underlying portion of intramedullary implant 70.

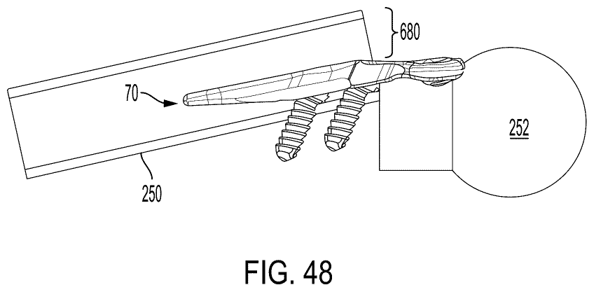

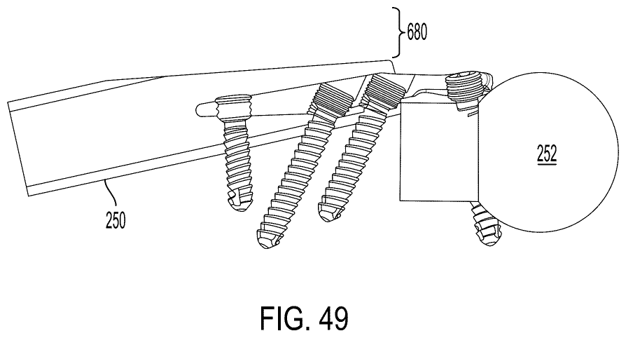

After realignment of distal metatarsal bone portion 252 relative to proximal metatarsal bone portion 250 and installation of intramedullary implant 70 along with insertion of any corresponding fixation members, the procedure may be substantially complete. Because distal metatarsal bone portion 252 is shifted relative to proximal metatarsal bone portion 250 to offset the cut end faces of the two bone portions, a section of the cut end face of proximal metatarsal bone portion may project medially from intramedullary implant 70 and/or distal metatarsal bone portion 252. This can present a residual medial aspect or bone spike that the clinician may remove to remove the medial prominence. FIG. 48 illustrates an example arrangement of proximal metatarsal bone portion 250 relative to distal metatarsal bone portion 252 with intramedullary implant 70 installed and shows an example medial projection 680 of the proximal metatarsal bone portion that may desirably be removed. FIG. 49 illustrates an example removal profile of the medial projection 680 of the proximal metatarsal bone portion 250.

To remove the medial projection 680, a clinician can guide a cutting instrument at an angle relative to the longitudinal shaft of proximal metatarsal bone portion 250 (e.g., parallel to the face of the stem portion of intramedullary implant 70) to remove the medial projection. Example cutting instruments that can be used include a saw blade, a drill, a burr, an osteotome, and/or another instrument operable to remove the bone region. The clinician can guide the cutting instrument freehand to remove medial projection 680 or may use a guide to guide the cutting instrument.

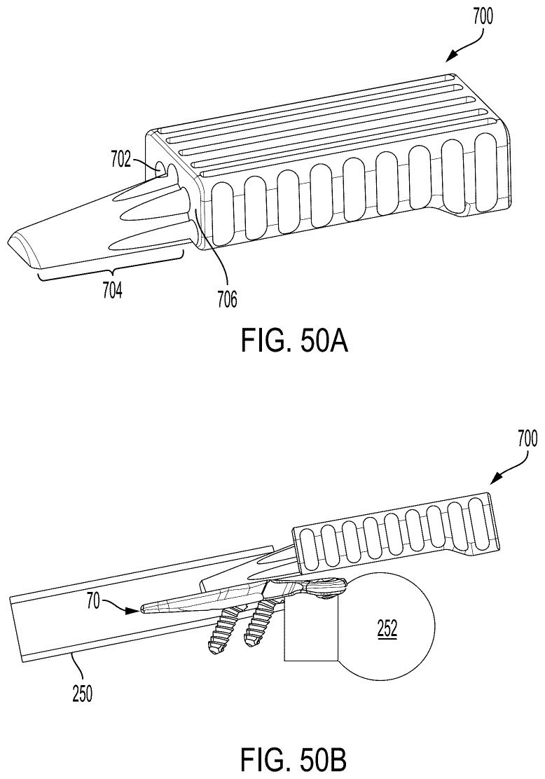

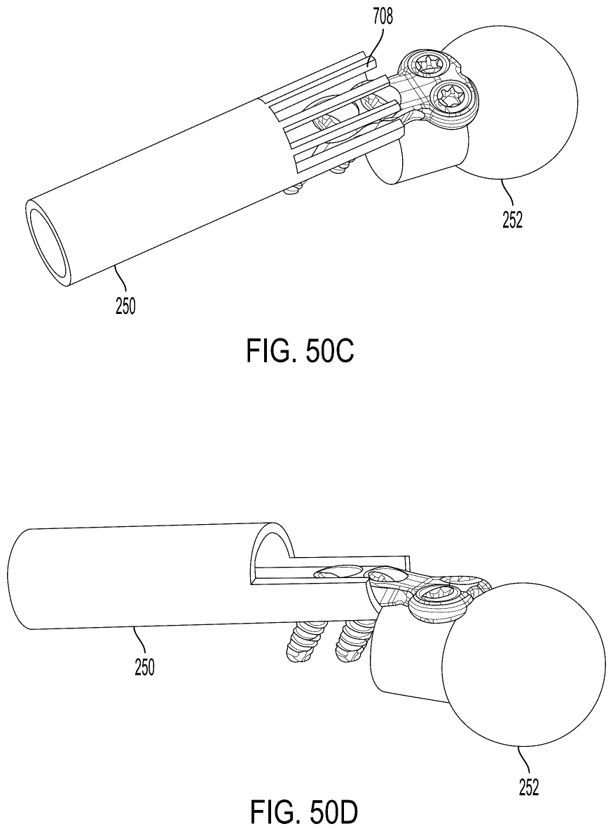

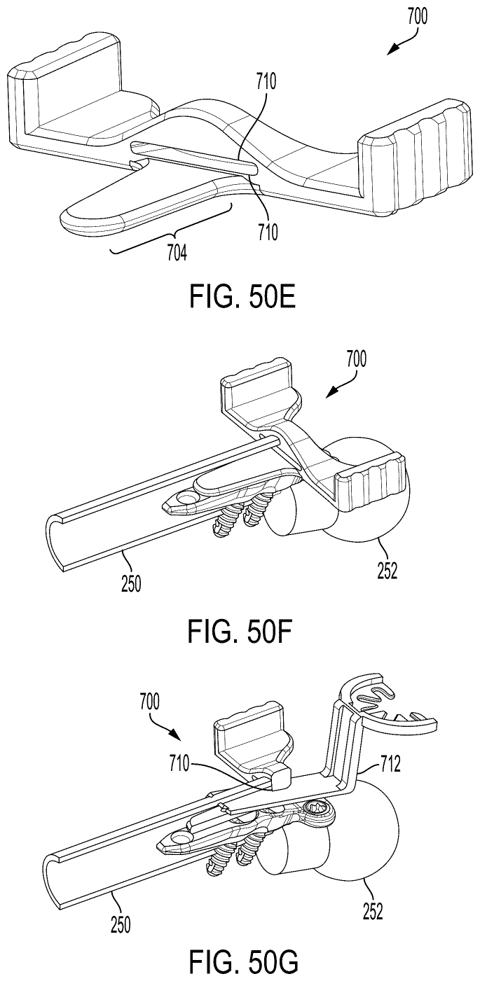

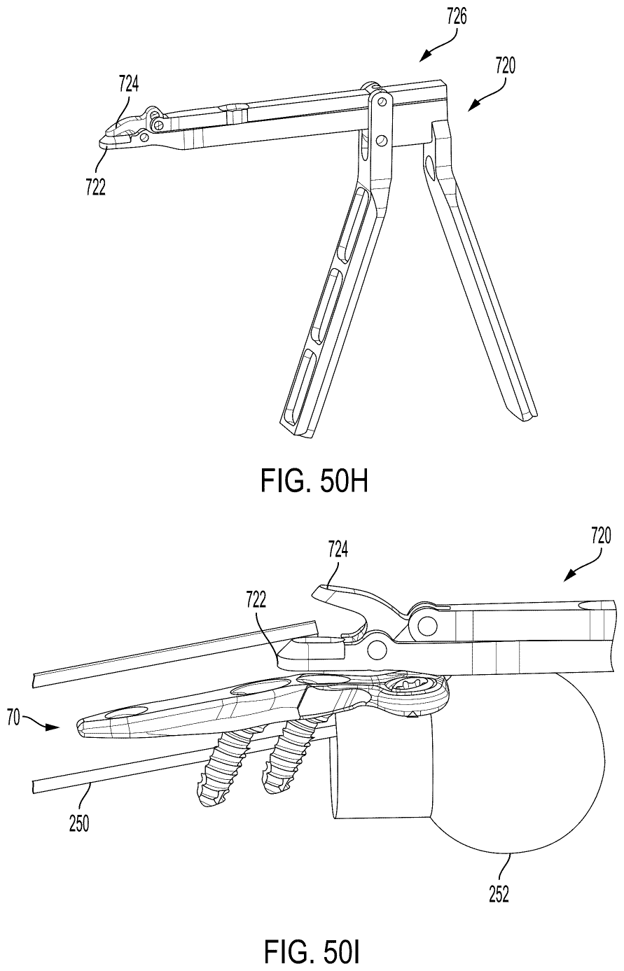

FIGS. 50A-50I illustrate example guides and associated procedural steps that can be used to guide removal of the medial projection 680 of proximal metatarsal bone portion 250. FIG. 50A illustrates an example guide 700 having multiple holes 702 extending the length of the guide for guiding a drill bit along the length of the guide. Guide 700 includes an insertion region 704 configured to be inserted through the cut end face of proximal metatarsal bone portion 250 to orient the guide features of guide 700 relative to medial projection 680. Guide 700 can include a shelf or stop 706 that limits how far the guide and/or projection can be inserted into the medullary canal of proximal metatarsal bone portion 250. The location of stop 706 may be fixed or may be adjustable to provide configurability of guide 700 depending on the anatomy of the patient being operated upon.

FIG. 50B illustrates insertion region 704 of guide 700 inserted through the cut end face of proximal metatarsal bone portion 252 to align one or more guide surfaces, such as one or holes 702 for guiding a cutting instrument such as a drill bit, for removing medial projection 680. FIG. 50C illustrates proximal metatarsal bone portion 250 after a drill bit has been guided through holes 702 to remove regions of bone defining medial projection 680. When using a drill bit that is guided through holes spaced apart from each other, residual slivers of bone 708 may be present that can be broken or cut off and removed, for example using an osteotome or another instrument, to remove the medial projection as shown in FIG. 50D.

FIG. 50E illustrates another example configuration of a guide 700 configured to guide a cutting instrument for cutting medial projection 680. In this example, guide 700 defines one or guide surfaces 710, such as a pair of opposed guide surfaces defining a slot there between. In this example, guide 700 may also define a body portion or region 704 configured to be inserted through the cut end face of proximal metatarsal portion 252 position and orient the one or more guide surfaces 710 relative to medial projection 680 to be removed. FIG. 50F illustrates guide 700 from FIG. 50E inserted through the cut end face of proximal metatarsal portion 252. FIG. 50G illustrates an example cutting instrument 712 being guided along the one or more guide surfaces 710 to cut and remove medial projection 680. In this example, cutting instrument 712 is illustrated as a saw blade, although other cutting instruments can be used in guided the guide 700 without departing from the scope of disclosure.

FIG. 50H illustrates another bone removing instrument 720 that can be used to remove medial projection 680. Instrument 720 is illustrated as a cutting or clipping instrument having a pair of opposed bodies 722 and 724 defining a jaw between the opposed bodies. The opposed bodies 722, 724 can be connected via a mechanical linkage 726 to an actuator that moves the bodies relative to each other to open and close the jaw. Instrument 720 may be sized and configured to insert one of the bodies 722 through the cut end face of proximal metatarsal bone portion 250 with the other of the bodies 724 being locatable on the opposite side of the cortical wall of the bone portion, as illustrated FIG. 50I. The bodies can be moved relative to each other to apply a cutting force effective to cut and remove a region of bone defining medial projection 680. Bodies 722, 724 may have sharpened surfaces for cutting teeth effective to cut through the wall of the bone. Depending on the size and configuration of the bodies, the clinician may actuate instrument 720 a single time to remove the entirety of medial projection 684 may make a series of smaller cuts and progressively advance instrument 720 proximally from the cut end face of proximal metatarsal bone portion 250 through remove the desired amount of bone.

Systems and techniques described herein can provide a variety of features and functionalities beneficial for patients undergoing a metatarsal treatment procedure and clinicians performing such a procedure. In some examples, a system includes an intramedullary implant insertion and positioning instrument and an intramedullary implant configured to be releasably coupled to the instrument. The intramedullary implant can have a variety of configurations, including a plate portion having at least two fixation apertures and a stem portion having at least one fixation aperture (e.g., one, two, three, or more). The fixation apertures of the intramedullary implant may all be threaded and configured to receive locking screws (e.g., with screw openings drilled and screws inserted through screw guide openings defined by the instrument). The metatarsal of the patient may be accessed through a single small incision through the skin of the patient (e.g., 2.5 mm or less, such as 2.0 mm or less, 1.75 mm or less, or 1.5 mm or less) through which the metatarsal is cut and the screws are inserted into the plate portion and the stem portion of the intramedullary implant. For example, the clinician may insert two screws into the plate portion of the implant through the single incision and may also insert one, two, or more screws into the stem portion of the implant. One or more screws located farther distally along the length of the implant portion (e.g., one or more tail screws) may be inserted through one or more percutaneous poke incisions through the skin of the patient offset from the single comparatively larger incision.

The intramedullary implant insertion and positioning instrument may include a first bone positioning device 162 which is configured to move distal metatarsal bone portion 252 relative to proximal metatarsal bone portion 250. First bone positioning device 130, 150 can rotate and/or change the angle of intramedullary implant 70 from an initial insertion angle to orient the intramedullary implant 70 to be substantially parallel to the longitudinal axis of proximal metatarsal bone portion 250. The intramedullary implant insertion and positioning instrument may include additionally or alternatively include a second bone positioning device 360, 380, 450 which is configured to move distal metatarsal bone portion 252 relative to proximal metatarsal bone portion 250 in the frontal plane and/or sagittal plane.

The intramedullary implant insertion and positioning instrument may include additionally or alternatively include an intramedullary insertion body 306 operatively connected to the instrument. The intramedullary implant 70 can move relative to intramedullary insertion body 306, e.g., by moving the intramedullary insertion body 306 relative to the intramedullary implant releasably coupled at a fixed location to the instrument. The intramedullary insertion body 306 and intramedullary implant 70 can move relative to each other, e.g., causing the intramedullary insertion body to apply a pulling force to a proximal metatarsal bone portion 250 (e.g., to an internal cortical wall such as a medial internal cortical wall of the bone portion) and/or the plate portion of intramedullary implant 70 to apply a pushing force to the distal metatarsal bone portion 252 (e.g., the bone-contacting surface of the plate portion to push against an external cortical wall such as a medial external cortical wall of the bone portion).

In use, the stem portion of intramedullary implant 70 can be inserted into proximal metatarsal bone portion 250. The proximal bone positioner 162 can be engaged, e.g., by moving a proximal positioning cup or other contact surface to apply a force to change the orientation of intramedullary implant 70 in proximal metatarsal bone portion 250, causing the intramedullary implant to rotate from being at a first angular orientation in proximal metatarsal bone portion 250 to a second angular orientation (e.g., substantially co-axial aligned with the longitudinal axis of proximal metatarsal bone portion 250). This can also involve the distal end of intramedullary implant 70 moving laterally within proximal metatarsal bone portion 250. Engagement of the proximal bone positioner 162 can additionally or alternatively cause the plate portion of intramedullary implant 70 contacting the surface of distal metatarsal bone portion 252 to cause the distal metatarsal bone portion 252 to translate laterally in the transverse plane.

Before, during, and/or after engaging proximal bone positioner 162, the clinician can move intramedullary insertion body 306 relative to intramedullary implant 70. This can provide further fine tunned control over the transverse plane positioning of distal metatarsal bone portion 252 and/or help stabilize attachment of the instrument to the bone portions. Movement of intramedullary insertion body 306 relative to intramedullary implant 70 can apply a force causing the plate portion of intramedullary implant 70 contacting the surface of distal metatarsal bone portion 252 to push the distal metatarsal bone portion 252 laterally in the transverse plane and/or pull the proximal metatarsal bone portion 250 medially in the transverse plane. Movement of intramedullary insertion body 306 relative to intramedullary implant 70 can cause the distance between the intramedullary insertion body and intramedullary implant to increase (e.g., increasing the distance between the intramedullary insertion body contacting the medial cortex of the proximal metatarsal bone portion) and the plate portion contacting the distal metatarsal bone portion).

Before and/or after engaging proximal bone positioner 162 to move distal metatarsal portion 252 and/or to adjust the angular orientation of intramedullary implant 70 and/or moving intramedullary insertion body 306 relative to intramedullary implant 70, the clinician may insert one or more provisional fixation wires to temporarily hold a provisional moved position (e.g., in the transverse plane) of one or both bone portions. In various examples, the clinician may insert a fixation wire through an opening extending through bone positioner 162 and/or through an adjacent opening offset from a shaft of bone positioner 162. The fixation wire may help fixate a position of the instrument to the underlying bone portion(s). The fixation wire(s) may extend only into the underlying bone portion(s) or may extend through the bone portion(s) and into an adjacent bone (e.g., second metatarsal 214).

Before, during, and/or after engaging proximal bone positioner 162 and/or moving intramedullary insertion body 306 relative to intramedullary implant 70, the clinician may use a bone positioning device 360, 380, 450 to move distal metatarsal bone portion 252 relative to proximal metatarsal bone portion 250 in the frontal plane and/or sagittal plane. The bone positioning device may be actuated to cause a rotation force to be applied on a pin place in distal metatarsal portion 252. The pin may be comparatively large (e.g., have a diameter of 2.2 mm or greater) to accommodate the force applied to the pin. By placing the pin generally perpendicular to the long axis of the first metatarsal 212 initially, the force applied to the pin may more directly translate to frontal plane rotation than when the device guide pushes on a pin inserted at a different angle, which may promote both frontal plane rotation and lateral shifting. Additionally or alternatively, the clinician can control sagittal plane positioning, e.g., by moving the pin dorsally or plantarly and/or by adjusting the angle of the pin in the proximal to dorsal direction to (e.g., moving the pin to one of multiple different proximal-distal notches in the device) to rotates distal metatarsal bone portion 252 in the sagittal plane. Bone positioning device 360, 380, 450 and/or one or more other features of the instrument may be plastic/radiolucent to facilitate improved fluoroscopy visualization.

Any of the instruments, devices, and/or implants described herein can be designed and constructed with patient-specific sizing and/or characteristics (e.g., one or more characteristics configured to interface with patient-specific anatomical attributes). In these examples, the anatomical characteristics (e.g., size and/or shape) of at least a portion of the patient's foot undergoing the procedure can be determined prior to performing the surgical procedure. The patient's foot may be imaged to provide data indicative of the size and structure of the patient's foot. A computational model representative of the patient's foot may then be generated and one or more of the instruments and/or implants to be used during the procedure sized, shaped, and/or otherwise configured to the specific anatomical characteristics of the foot of the patient undergoing the procedure. The instruments and/or implants can then be manufactured to provide one or more patient-specific components that are then used during the subsequent surgical procedure. For example, the instruments and/or implants may have one or more surface features size and shape indexed to corresponding anatomical location(s) of the patient's bone where the features can be positioned.

Various examples have been described. These and other examples are within the scope of the following claims.

1. An intramedullary implant insertion and positioning instrument, the instrument comprising:
a body configured to releasably connect to an intramedullary implant having a stem portion insertable into a first metatarsal portion and a plate portion positionable against a second metatarsal portion; and
an intramedullary insertion body configured to be inserted into a medullary canal of the first metatarsal portion, the intramedullary insertion body being operatively connected to the body, and the intramedullary insertion body and the intramedullary implant being movable relative to each other, when the intramedullary implant is releasably connected to the body.

2. The instrument of claim 1, wherein the intramedullary insertion body and the intramedullary implant are movable relative to each other, when the intramedullary implant is releasably connected to the body, to apply a force pulling the first metatarsal portion toward the body while the plate portion of the intramedullary implant pushes against the second metatarsal portion.

3. The instrument of claim 1, wherein the intramedullary implant is configured to be releasably connected to the body at a fixed position relative to the body and the intramedullary insertion body is configured to move relative to the intramedullary implant connected at the fixed position.

4. The instrument of claim 1, wherein the intramedullary insertion body and the intramedullary implant are movable relative to each other from a position in which the intramedullary insertion body is adjacent to the intramedullary implant, when the intramedullary implant is releasably connected to the body, to a position in which the intramedullary insertion body is spaced from the intramedullary implant.

5. The instrument of claim 1, further comprising a bone positioning device operatively connected to the body, the bone positioning device being configured to apply a force between the body and a bone portion underlying the body.

6. The instrument of claim 5, wherein the bone positioning device comprises a contact surface operatively coupled to the body via a shaft, and the shaft is configured to translate relative to the body.

7. The instrument of claim 5, wherein the bone portion underlying the body comprises the first metatarsal portion.

8. The instrument of claim 1, further comprising an intramedullary insertion body translation mechanism configured to control movement of the intramedullary insertion body relative to the body.

9. The instrument of claim 1, wherein the intramedullary insertion body is detachable from the body and replaceable from a system of different intramedullary insertion bodies.

10. The instrument of claim 1, wherein:
the intramedullary insertion body defines an opening extending through the body; and
the body defines at least one screw insertion aperture that is positioned to be co-axially aligned with both (1) the opening extending through the intramedullary insertion body and (2) a screw aperture of the intramedullary implant.

11. The instrument of claim 10, further comprising an attachment rod configured to releasably connect the intramedullary implant to the body with the attachment rod inserted through the screw insertion aperture of the body, through the opening extending through the intramedullary insertion body, and being threadingly engaged with the screw aperture of the intramedullary implant.

12. The instrument of claim 1, wherein the body comprises an intramedullary implant receiving surface against which the intramedullary implant is configured to positioned and releasably connected, and the intramedullary implant receiving surface comprises a hook configured to wrap at least partially about a thickness of the intramedullary implant, when the intramedullary implant is releasably connected to the body.

13. The instrument of claim 1, wherein the first metatarsal portion is a proximal metatarsal portion and the second metatarsal portion is a distal metatarsal portion.

14. A method comprising:
cutting a metatarsal bone of a foot into a first metatarsal portion and a second metatarsal portion;
inserting a stem portion of an intramedullary implant releasably connected to a body of an instrument and an intramedullary insertion body connected to the body of the instrument into a medullary canal of the first metatarsal portion;
positioning a plate portion of the intramedullary implant overlying the second metatarsal portion;
moving the intramedullary insertion body relative to the intramedullary implant to apply a force to the first metatarsal portion; and
inserting a fixation member through a fixation aperture extending through the plate portion of the intramedullary implant and into the second metatarsal portion to fixate a moved position of the second metatarsal portion relative to the first metatarsal portion.

15. The method of claim 14, wherein moving the intramedullary insertion body relative to the intramedullary implant to apply the force comprises pulling the first metatarsal portion toward the body of the instrument while the plate portion of the intramedullary implant pushes against the second metatarsal portion.

16. The method of claim 14, wherein moving the intramedullary insertion body relative to the intramedullary implant to apply the force to the first metatarsal portion comprises pushing the plate portion of the intramedullary implant against the second metatarsal portion to move the second metatarsal portion laterally in a transverse plane.

17. The method of claim 14, wherein the intramedullary implant is releasably connected to the body at a fixed position relative to the body, the intramedullary insertion body is movable relative to the intramedullary implant connected at the fixed position, and moving the intramedullary insertion body relative to the intramedullary implant to apply the force comprises moving the intramedullary insertion body relative to the intramedullary implant connected at the fixed position.

18. The method of claim 14, wherein:
inserting the stem portion of the intramedullary implant and the intramedullary insertion body into the medullary canal of the first metatarsal portion comprises inserting the stem portion of the intramedullary implant and the intramedullary insertion body into the medullary canal of the first metatarsal portion with the intramedullary insertion body positioned adjacent to the intramedullary implant; and
moving the intramedullary insertion body relative to the intramedullary implant comprises moving the intramedullary insertion body and the intramedullary implant away from each other.

19. The method of claim 14, wherein the intramedullary implant is releasably connected to the body of the instrument with a longitudinal axis of the intramedullary implant positioned parallel to the intramedullary insertion body.

20. The method of claim 14, wherein the instrument further comprises a bone positioning device operatively connected to the body of the instrument, and further comprising using the bone positioning device to apply a force to the first metatarsal portion at a location offset from a location where the intramedullary insertion body and the intramedullary implant move relative to each other.

21. The method of claim 20, wherein using the bone positioning device to apply the force to the first metatarsal portion comprises applying a laterally-directed pushing force to the first metatarsal portion and thereby causing the plate portion of the intramedullary implant to push the second metatarsal portion laterally.

22. The method of claim 20, wherein:
moving the intramedullary insertion body relative to the intramedullary implant to apply the force to the first metatarsal portion comprises moving the intramedullary insertion body relative to the intramedullary implant to apply a medial-directed pulling force to the first metatarsal portion; and
using the bone positioning device to apply the force to the first metatarsal portion comprises applying a laterally-directed pushing force to the first metatarsal portion.

23. The method of claim 22, wherein:
the medial-directed pulling force is applied against an internal cortical wall surface of the first metatarsal portion; and
the laterally-directed pushing force is applied against an external cortical wall surface of the first metatarsal portion.

24. The method of claim 14, wherein the body comprises an intramedullary implant receiving surface against which the intramedullary implant is configured to positioned and releasably connected, the intramedullary implant receiving surface comprises a hook wrapping at least partially about a thickness of the plate portion of the intramedullary implant.

25. The method of claim 14, wherein:
the stem portion of the intramedullary implant comprises a screw aperture; and
the body of the instrument is releasably connected to the intramedullary implant with an attachment rod inserted through a screw insertion aperture of the body with the attachment rod threadingly engaged with the screw aperture of the intramedullary implant.

26. The method of claim 14, wherein inserting the fixation member through the fixation aperture extending through the plate portion of the intramedullary implant comprises guiding the fixation member through a screw insertion aperture defined by the body of the instrument that is co-axially aligned with the fixation aperture extending through the plate portion of the intramedullary implant.

27. The method of claim 14, further comprising inserting a fixation member through a fixation aperture extending through the stem portion of the intramedullary implant and into the first metatarsal portion.

28. The method of claim 27, wherein the fixation aperture extending through the stem portion comprises a fixation aperture located in a proximal-most half of the stem portion.

29. The method of claim 14, wherein the first metatarsal portion is a proximal metatarsal portion and the second metatarsal portion is a distal metatarsal portion.
